(19) United States
(12) Patent Application Publication
(10) Pub. No.: US 2020/0297619 A1
(43) Pub. Date: Sep. 24, 2020

(54) BIOME-FRIENDLY CONSUMER PRODUCTS

(71) Applicant: AOBIOME LLC, Cambridge, MA (US)

(72) Inventors: Lauren Nicole Ambrogio, Boulder, CO (US); James Hoffman, Woodstock, NY (US); Larry Weiss, San Francisco, CA (US)

(21) Appl. No.: 16/723,028

(22) Filed: Dec. 20, 2019

Related U.S. Application Data
(60) Provisional application No. 62/784,601, filed on Dec. 24, 2018.

(51) Int. Cl. A61K 8/99; C11D 3/38; A61Q 19/10; A61Q 5/02; A61Q 5/12

(52) U.S. Cl. CPC A61K 8/99; C11D 3/381; A61Q 19/10; A61K 2800/70; A61Q 5/12; A61K 2800/10; A61K 2800/30; A61Q 5/02

(57) ABSTRACT

The systems and methods of the disclosure provide, inter alia, consumer products, e.g., finished consumer products that may be considered to be “biome-friendly” or “biome-compatible.” The systems and methods of the disclosure may provide for use of consumer products, e.g., finished consumer products, that may be used in combination with bacteria, e.g., non-pathogenic bacteria, e.g., ammonia oxidizing bacteria, which may be used in the form of a preparation or composition to be applied to a surface and/or to be used by a subject.

### CROSS-REFERENCE TO RELATED APPLICATIONS

This application claims priority under 35 U.S.C. § 119(e) to U.S. Provisional Application No. 62/784,601 titled “BIOME-FRIENDLY CONSUMER PRODUCTS,” filed Dec. 24, 2018, which is incorporated herein by reference in its entirety for all purposes.

### BACKGROUND

Beneficial bacteria, e.g., non-pathogenic bacteria, can be used to suppress the growth of pathogenic bacteria. Bacteria and other microorganisms are ubiquitous in the environment and are naturally present on subjects, for example, human and animal subjects. Bacteria are a normal part of the environment of all living things. In the gut, these bacteria are not pathogenic under normal conditions, and in fact improve health by rendering the normal intestinal contents less hospitable for disease causing organisms. Disease prevention is accomplished in a number of ways: nutrients are consumed, leaving less for pathogens; conditions are produced, such as pH and oxygen tension, which are not hospitable for pathogens; compounds are produced that are toxic to pathogens; pathogens are consumed as food by these microorganisms; less physical space remains available for pathogens; and specific binding sites are occupied leaving fewer binding sites available for pathogens. The presence of desirable bacteria is seen as useful in preventing disease states.

There is a need in the art to provide products, e.g., consumer products, that maintain or sustain at least some level of beneficial bacteria on or in subjects. This may be accomplished so that beneficial bacteria may regulate or suppress the growth of non-autotrophic bacteria, e.g., pathogenic bacteria, and/or maintain a suitable microbiome on a subject to benefit from health improvements that the non-pathogenic bacteria may provide.

### SUMMARY

There is provided a biome-friendly consumer product. The biome-friendly consumer product may comprise ammonia oxidizing microorganisms (AOM). The biome-friendly consumer product may be supportive of a microbiome of a subject.

In accordance with certain embodiments, the biome-friendly consumer product comprising AOM may, in the absence of treatment, e.g., sterilization treatment or the addition of a preservative, support microbe growth, e.g., bacterial or fungal growth, e.g., as measured by U.S.P. 51, Antimicrobial Effectiveness Testing or U.S.P. 62, Microbial Examination of Nonsterile Products.

In accordance with certain embodiments, the biome-friendly consumer product comprising AOM may, if exposed to challenge with a microbe, e.g., a bacterium or fungus, support growth of said microbe, e.g., as determined by U.S.P. 51, Antimicrobial Effectiveness Testing or U.S.P. 62, Microbial Examination of Nonsterile Products.

In some embodiments, the consumer product allows the microbiome of a subject to be maintained. The consumer product may be substantially free of other microorganisms. The consumer product may comprise a community of microorganisms, e.g., a community of beneficial microorganisms.

In some embodiments the consumer product may be formulated to provide a health benefit or otherwise support a healthy lifestyle. The consumer product may be formulated to provide a health benefit to at least one of the skin, scalp, gastrointestinal system, and respiratory system of the subject. The consumer product may be formulated support engraftment of the AOM. The consumer product may be configured, e.g., structured, to support engraftment of the AOM.

In some embodiments the consumer product may be treated to provide a health benefit or otherwise support a healthy lifestyle. The consumer product may be treated to provide a health benefit to at least one of the skin, scalp, gastrointestinal system, and respiratory system of the subject. The consumer product may be treated support engraftment of the AOM.

The consumer product may be formulated to mitigate or modify odor.

In accordance with certain embodiments, the consumer product is a household product. The household product may be an air freshener, air conditioning product, e.g. anti-mold air conditioning product, surface conditioner, deodorizer, surface cleaner, glass cleaner, bathroom cleaner, e.g., toilet or shower cleaner, appliance, vacuum, storage or organization device, home improvement device, pest control product, dinnerware, serveware, glassware, drinkware, cookware, bakeware, utensil, cutlery, kitchen tool, kitchen cleaner, furniture, furniture cleaner, floor or carpet cleaner, disinfectant, soap, trash bag, grocery, food storage bag or container, cooler, water bottle, disposable tableware, dishwashing product, car cleaning or detailing product, towel, linen, window treatment, media, e.g. a book, music or movie, sport or outdoor recreational equipment, exercise or fitness equipment, sponge, lamp, light, rug, luggage, scrubber, dusting product, lawn, garden, patio, outdoor cooking, footwear conditioning product, cotton product, e.g. cotton bud, or paper product, e.g. toilet paper, facial tissue, or paper towel.

In accordance with certain embodiments, the consumer product is a laundry product. The laundry product may be, e.g., laundry detergent, bleach, dryer sheet, scent, post-laundry finishing spray, stain remover, or fabric softener. The consumer product may be a fabric or textile.

In accordance with certain embodiments, the consumer product is a baby product. The baby product may be, e.g., a car seat, stroller, carrier, nursery, diapering, nursing, bathroom, toy, training, or infant safety product or accessory. In at least some embodiments, the baby product may be a bottle, baby bathing product, pacifier, teething product, crib, bassinet, swing, utensil, cup, plate, storage, clothing, or diaper product.

In accordance with certain embodiments, the consumer product is a pet product. The pet product may be, e.g., a pet health, safety, training, cleaning, grooming, bathing, pest management, crate, carrier, bowl, collar, harness, leash, bed, accessory, storage container (i.e. non-kennel or non-crate container), litterbox, litter, diaper pad, toy, or treat product.

In accordance with certain embodiments, the consumer product may be a food or beverage product. The consumer product may be a dietary or nutritional supplement, i.e. a vitamin, mineral, specialty (e.g. fish oil, fiber, probiotic, prebiotic, glucosamine, chrondroitin, CoQ10), herbal, botanical, sports, or weight management supplement.

In accordance with certain embodiments, the consumer product may be a personal product.

In accordance with certain embodiments, the consumer product may be an electronic device and/or a component thereof. The electronic device may be, e.g., a television, video game system, audio system, headphone, mobile phone, wearable device, tablet, e-reader, or a technology accessory, i.e. a mobile technology accessory.

In accordance with certain embodiments, the consumer product may be an accessory. The accessory may be, e.g., a handbag, wallet, keychain, hat, scarf, belt, glove, mitten, jewelry, hair accessory, glasses, or sunglasses. In at least some embodiments, the accessory is a personal accessory.

In accordance with certain embodiments, the consumer product may be an office or school supply.

The consumer product may be substantially free of a preservative. The consumer product may have less than about 500 ppb of a preservative listed in Annex VI, e.g., less than 500 ppb of a preservative listed in Annex VI.

In some embodiments, the consumer product may be irradiated or otherwise sterilized. On the consumer product, at least 10, 20, 30, 40, 50, 60, 70, 80, 90, 95, 99, or 99.9% of any microorganisms present may be dead, incapable of cell division, or have radiation induced DNA damage sufficient to inhibit cell division. The consumer product may include an indicator of heating, sterilization, or irradiation.

The consumer product may be treated to favor at least one pre-determined species of microorganism, e.g., the AOM. The consumer product may be treated to suppress growth or reproduction of at least one pre-determined species of microorganism. The consumer product may be treated to have anti-fungal properties. The consumer product may be treated to have anti-viral properties.

In accordance with certain embodiments, the consumer product may be associated with a deterioration-based or a biome-compatible-based expiration date. The consumer product may comprise an indication of expiration, or lifetime, e.g., recommended lifetime, after a preselected period of time, e.g., expressed in days, that is less than X days from the date of one of manufacturing, filling, sealing, shipping, releasing into commerce, or selling, wherein X is about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days.

The consumer product may further comprise a component added to provide one or more of the following: a fragrance, a color, viscosity, foam forming and foam stability, adhesion, moisture retention, moisture binding, pH stabilization, cleansing, thickening, softening, conditioning, e.g., hair or skin conditioning, lipid layer enhancing, barrier-forming, or film-forming. The consumer product may comprise one or more of an antioxidant, fatty substance/oil, thickener, softener, emulsifier, light-screening agent, foam forming and foam stability, antifoaming agent, moisturizer, fragrance, surfactant, filler, sequestering agent, polymers, acidifying or basifying agent, dyes, colorant, pigment, pearlizer, opacifier, organic or inorganic particle, viscosity modifier, cleanser, adherent, moisture binder, pH stabilizer, conditioner, de-tangler, biobased surfactant cleanser, lipid layer enhancer, skin conditioner, and natural hair nutrient such as botanicals, fruit extracts, sugar derivatives and/or amino acids, hydrolyzed proteins, or vitamins. The consumer product may comprise one or more of water, cocamidopropyl betaine, rosa damascena flower water, decyl glucoside, pyrus malus (apple) fruit extract, glycerin, hydrolyzed adansonia digitata (baobab) seed protein, and hydroxypropylcellulose. The consumer product may comprise one or more of water, ammonia, decyl glucoside, lauryl glucoside, sodium citrate, lauramine oxide, polysorbate 20, glycerin, disodium 2-sulfolaurate, sodium methyl 2-sulfolaureate, tetrasodium glutamate diacetate, sodium sulfate, laureth-7, propanediol, gluconolactone, xanthan gum, propylene glycol, sodium benzoate, sorbitol, calcium chloride, protease, amylase, lipase, mannase, sodium formate, pectase, lyase, or calcium bromide.

The consumer product may be disposed in an end-use container.

In accordance with another aspect, there is provided a kit comprising a biome-friendly consumer product according to any of the preceding claims and a preparation comprising ammonia oxidizing microorganisms (AOM). The preparation may comprise a community of microorganisms, e.g., beneficial microorganisms.

The kit may further comprise a probiotic preparation. The kit may further comprise a prebiotic preparation.

The preparation may be substantially free of other organisms.

In accordance with certain embodiments, the preparation may comprise between about 1×10³ CFU/mL to about 1×10¹⁴ CFU/mL AOM. The preparation may comprise between about 1×10⁹ CFU/mL to about 10×10⁹ CFU/mL AOM.

The AOM may comprise ammonia oxidizing bacteria (AOB). The AOM may consist essentially of AOB. The AOM may consist of AOB. The AOM may comprise Nitrosomonas, Nitrosococcus, Nitrosospira, Nitrosocystis, Nitrosolobus, Nitrosovibrio, and combinations thereof.

In certain embodiments, the AOM may be Nitrosomonas eutropha (N. eutropha). The AOM may be N. eutropha D23, having ATCC accession number PTA-121157.

The AOM may comprise ammonia oxidizing archaea (AOA).

In accordance with certain embodiments, the AOM may be capable of converting ammonia or ammonium to nitrite at a rate of at least about 1 pmol/min/mg protein, e.g., at least about 0.1 nmol/min/mg protein.

The preparation may comprise AOM in a buffer solution, e.g., aqueous buffer solution, comprising disodium phosphate and magnesium chloride, for example, 50 mM Na₂HPO₄ and 2 mM MgCl₂ in water.

The kit may further comprise instructions directed to combined use of the biome-friendly consumer product and the preparation to impart a cosmetic or a therapeutic effect in a subject.

In some embodiments, the preparation may be configured to be administered to the subject topically, orally, enterally, intranasally, parenterally, subcutaneously, ocularly, otically, or respiratorilly.

The preparation may be packaged for single or multiple use. The preparation may be a spray, aerosol, or mist.

In accordance with another embodiment, there is provided a method of supporting a microbiome of a subject. The method may comprise introducing a consumer product to the subject. The method may comprise introducing a source of ammonia oxidizing microorganisms (AOM) to the subject.

The method may comprise introducing the source of AOM to the subject concurrently with the consumer product. The method may comprise introducing the source of AOM to the subject after introducing the consumer product. The method may comprise introducing a second or subsequent dose of the source of AOM to the subject.

In accordance with certain embodiments, introducing the consumer product involves use thereof in an environment of the subject.

The method may further comprise a second or subsequent use of the consumer product in an environment of the subject.

In accordance with another aspect, there is provided a method of making a biome-friendly consumer product. The method may comprise selecting a first component from a list of biome-friendly components. The method may comprise selecting a second component from a list of biome-friendly components. The method may further comprise integrating the first and second components with a source of ammonia oxidizing microorganisms (AOM).

In accordance with another aspect, there is provided a method of making a biome-friendly consumer product the method may comprise providing first and second components, wherein each has been shown to be biome-friendly. The method may comprise providing a source of ammonia oxidizing microorganisms (AOM). The method may comprise integrating said first and second components with the source of AOM to form a consumer product. The method may further comprise determining if the product is biome-friendly, thereby making the biome-friendly consumer product.

In some embodiments, the method may comprise exposing the product to challenge with a microbe, e.g., a bacterium or fungus. The method may further comprise applying the product to a surface and exposing the surface to challenge with a microbe, e.g., a bacterium or fungus. The method may comprise determining whether the product will support growth of a microbe, e.g., a bacterium or fungus. The method may comprise determining whether the product will support growth of a microbe, e.g., a bacterium or fungus, by U.S.P. 51, Antimicrobial Effectiveness Testing or U.S.P. 62, Microbial Examination of Nonsterile Products.

In accordance with another aspect there is provided a method of evaluating a consumer product comprising ammonia oxidizing microorganisms (AOM). The method may comprise contacting a portion or aliquot of the consumer product with a test organism. The method may comprise evaluating an effect of the consumer product on the test organism. In some embodiments, evaluating comprises evaluating the effect of the consumer product on an ability of the test organism to breakdown organic waste or produce beneficial byproducts.

In accordance with another aspect, there is provided a method of rendering a consumer product biome-friendly. The method may comprise applying a biome-friendly composition to the consumer product. The method may further comprise applying a preparation comprising ammonia oxidizing microorganisms (AOM) to the consumer product.

In some embodiments, the method may further comprise re-applying the biome-friendly composition to the consumer product after an elapsed period of time. The method may comprise applying the biome-friendly composition to the consumer product concurrently with the preparation comprising AOM. The method may comprise applying the biome-friendly composition to the consumer product before applying the preparation comprising AOM. The method may comprise applying the biome-friendly composition to the consumer product after applying the preparation comprising AOM. The method may further comprise re-applying the preparation comprising AOM after an elapsed period of time.

In accordance with another aspect, there is provided a method of certifying a consumer product as being biome-friendly. The method may comprise providing a consumer product comprising ammonia oxidizing microorganisms (AOM). The method may comprise determining if the product is biome-friendly by exposing the product to challenge with a microbe, e.g., a bacterium or fungus. The method may further comprise certifying the product as being biome-friendly.

In some embodiments, the method may further comprise applying the product to a surface and exposing the surface to challenge with a microbe, e.g., a bacterium or fungus. The method may further comprise determining whether the product will support growth of a microbe, e.g., a bacterium or fungus. The method may comprise determining whether the product will support growth of a microbe, e.g., a bacterium or fungus, by U.S.P. 51, Antimicrobial Effectiveness Testing or U.S.P. 62, Microbial Examination of Nonsterile Products. The method may comprise contacting the consumer product with a test organism and evaluating the effect of the consumer product on an ability of the test organism to breakdown organic waste or produce beneficial byproducts.

In accordance with another aspect, there is provided a kit comprising a biome-friendly consumer product and a preparation comprising the test organism. The kit may further comprise instructions to evaluate the effect of the consumer product on the test organism.

In some embodiments, the test organism may comprise ammonia oxidizing microorganisms (AOM).

The kit may further comprise a reagent to detect nitrite production.

### BRIEF DESCRIPTION OF FIGURES

FIG. 1 is a table of a study of surfactants and their compatability with N. eutropha D23.

### DETAILED DESCRIPTION

The systems and methods of the disclosure provide, inter alia, consumer products, e.g., finished consumer products that may be considered to be “biome-friendly” or “biome-compatible.” The systems and methods of the disclosure may provide for use of consumer products, e.g., finished consumer products, that may be used in combination with bacteria, e.g., non-pathogenic bacteria, e.g., ammonia oxidizing bacteria, which may be used in the form of a preparation or composition to be applied to a subject.

Systems and methods of the disclosure may also provide for manufacturing of the consumer product, e.g., finished consumer product, quality control testing, and testing to determine the compatibility of a product, or one or more components of a product with bacteria, e.g., non-pathogenic bacteria, e.g., ammonia oxidizing bacteria, and or with a microbiome of a subject.

Systems and methods of the disclosure may provide for commercial release and distribution of the product, e.g., finished consumer product, into commerce, including methods of dispensing the product into commerce, refilling the product, and recycling the product.

### 1. Definitions

An ammonia oxidizing microorganism refers to a microorganism, e.g., bacterium capable of oxidizing ammonia or ammonium to nitrite and, under certain conditions, nitric oxide. This may be accomplished at a rate. The rate, e.g., a pre-determined rate, may refer to the conversion of ammonium ions (NH₄⁺) (e.g., at about 200 mM) to nitrite (NO₂⁻) at a rate of at least 50, 75, 125, or 150 micromoles NO₂⁻ per minute, e.g., about 100-150, 75-175, 75-125, 100-125, 125-150, or 125-175 micromoles/minute, e.g., about 125 micromoles NO₂⁻ per minute. In embodiments, the rate, e.g., a pre-determined rate, may refer to the conversion of ammonium ions (NH₄⁺) (e.g., at about 200 mM) to nitrite (NO₂⁻) at a rate of at least 50, 75, 125, or 150 nanomoles NO₂⁻ per minute per ml, e.g., about 100-150, 75-175, 75-125, 100-125, 125-150, or 125-175 nanomoles/minute/ml, e.g., about 125 nanomoles NO₂⁻ per minute per ml for a continuous culture, for example having an OD of about 0.5.

Examples of ammonia oxidizing bacteria include Nitrosomonas eutropha strains, e.g., D23 and C91, and other bacteria in the genera Nitrosomonas, Nitrosococcus, Nitrosospira, Nitrosocystis, Nitrosolobus, and Nitrosovibrio. D23 Nitrosomonas eutropha strain refers to the strain, designated AOB D23-100, deposited with the American Tissue Culture Collection (ATCC) (10801 University Blvd., Manassas, Va., USA) on Apr. 8, 2014 having accession number PTA-121157. The nucleic acid sequence(s), e.g., genome sequence, of accession number PTA-121157 are hereby incorporated by reference in their entireties. In certain embodiments, the N. eutropha is a strain described in PCT Application No. PCT/US2015/025909, filed Apr. 15, 2015, herein incorporated by reference in its entirety.

Optimized Nitrosomonas eutropha (N. eutropha), as that term is used herein, refers to an N. eutropha having an optimized growth rate; an optimized NH₄⁺ oxidation rate; or optimized resistance to NH₄⁺. In an embodiment it differs from naturally occurring N. eutropha by at least one nucleotide, e.g., a nucleotide in a gene selected from ammonia monooxygenase, hydroxylamine oxidoreductase, cytochrome c554, and cytochrome c_{M}552. The difference can arise, e.g., through selection of spontaneously arising mutation, induced mutation, or directed genetic engineering, of the N. eutropha. In an embodiment it differs from a naturally occurring N. eutropha in that it has a constellation of alleles, not present together in nature. These differences may provide for one or more of a treatment or prevention of a skin disorder, a treatment or prevention of a disease or condition associated with low nitrite levels, a treatment or prevention of body odor, a treatment to supply nitric oxide to a subject, and a treatment to inhibit microbial growth.

As used herein, a “beneficial microorganism” may include bacteria and archaea that are beneficial to a subject, e.g., to the health of a subject. The beneficial microorganisms may assist in metabolizing harmful or pathogenic compounds. The beneficial microorganisms may produce beneficial byproducts. In some embodiments, the beneficial microorganisms may assist in limiting or reducing a concentration of pathogenic microorganisms, for example, by exhausting a food source or product byproducts which are generally harmful to the pathogenic microorganisms.

As used herein, a “community of microorganisms” includes one or more microorganism. In some embodiments, the community of microorganisms may be a closed community. For example, the community of microorganisms may consist or consist essentially of selected microorganisms. In other embodiments, the community of microorganisms may be open. For example, the community of microorganisms may comprise selected microorganisms. The community of microorganisms may be 10%, 20%, 30%, 40%, 50%, 60%, 70%, 80%, 90%, 95%, 99%, 99.9%, or 99.99% composed of a selected or known group of species of microorganisms.

As used herein, an “autotroph”, e.g., an autotrophic bacterium, is any organism capable of self-nourishment by using inorganic materials as a source of nutrients and using photosynthesis or chemosynthesis as a source of energy. Autotrophic bacteria may synthesize organic compounds from carbon dioxide and ATP derived from other sources, coxiation of ammonia to nitrite, oxidation of hydrogen sulfide, and oxidation of Fe2⁺ to Fe3⁺ Autotrophic bacteria of the present disclosure are incapable of causing infection.

As used herein, “axenic” refers to a composition comprising an organism that is substantially free of other organisms. For example, an axenic culture of ammonia oxidizing bacteria is a culture that is substantially free of organisms other than ammonia oxidizing bacteria. An axenic composition may comprise elements that are not organisms, e.g., it may comprise nutrients or excipients. Any embodiment, preparation, composition, or formulation of ammonia oxidizing bacteria discussed herein may comprise, consist essentially of, or consist of optionally axenic ammonia oxidizing bacteria.

In some embodiments, “substantially free” denotes undetectable by a method used to detect the item that is indicated as “substantially free.” For example, “substantially free of a preservative” denotes that it is undetectable by a method used to detect a preservative. “Substantially free of microorganisms” denotes undetectable by a method used to detect other organisms, e.g., plating the culture and examining colony morphology, or PCR for a conserved gene such as 16S RNA.

A test, such as a minimum inhibitory concentration (MIC) test, may be performed. The MIC is defined as the lowest concentration of an anti-microbial that will inhibit the visible growth of a microorganism after incubation for a pre-determined period of time, for example, overnight incubation, for example, 12 hour, or 24 hour incubation.

To “culture” refers to a process of placing an amount of a desired bacterium under conditions that promote its growth, i.e., promoting cell division. The conditions can involve a specified culture medium, a set temperature range, and/or an agitation rate. Bacteria can be cultured in a liquid culture or on plates, e.g., agar plates.

The term “isolated,” as used herein, refers to material that is removed from its original or native environment (e.g., the natural environment if it is naturally occurring). For example, a naturally-occurring polynucleotide or polypeptide present in a living animal is not isolated, but the same polynucleotide or polypeptide, separated by human intervention from some or all of the co-existing materials in the natural system, is isolated. Such polynucleotides could be part of a vector and/or such polynucleotides or polypeptides could be part of a composition, and still be isolated in that such vector or composition is not part of the environment in which it is found in nature.

As used herein, the term “optimized growth rate” refers to one or more of: a doubling time of less than about 4, 5, 6, 7, 8, 9, or 10 hours when cultured under batch conditions as described in PCT/US2015/025909, filed Apr. 15, 2015; a doubling time of less than about 16, 18, 20, 22, 24, or 26 hours, when grown under chemostat conditions as described in PCT/US2015/025909, filed Apr. 15, 2015; or growing from an OD600 of about 0.15 to at least about 0.3, 0.4, 0.5, 0.6, 0.7, or 0.8 over about 1 or 2 days. In an embodiment, optimized growth rate is one having a doubling time that it is at least 10, 20, 30, 40, or 50% shorter than that of a naturally occurring N. eutropha.

As used herein, “optimized NH₄⁺ oxidation rate” refers to a rate of at least about 50, 75, 125, or 150 micromoles per minute of converting NH₃ or NH₄⁺ into NO₂⁻. For instance, the rate may be at least about 50, 75, 125, or 150 micromoles per minute of converting NH₄⁺ (e.g., at about 200 mM) to NO₂⁻. In an embodiment, an optimized NH₄⁺ oxidation rate is one in which NH₃ or NH₄⁺ is converted into NO₂⁻ at least 10, 20, 30, 40, or 50% more rapidly than is seen with a naturally occurring N. eutropha. In embodiments, the rate of at least about 50, 75, 125, or 150 nanomoles per minute per ml of converting NH₃ or NH₄⁺ into NO₂⁻. For instance, the rate may be at least about 50, 75, 125, or 150 nanomoles per minute per ml of converting NH₄⁺ (e.g., at about 200 mM) to NO₂⁻ for a continuous culture having an OD of about 0.5.

As used herein, “optimized resistance to NH₄⁺” refers to an ability to grow in conditions of greater than 50, 75, 100, 125, 150, 175, 200, 225, 250, 275, or 300 mM NH₃ or NH₄⁺ for at least about 24 or 48 hours. In an embodiment, an optimized resistance to NH₄⁺ refers to the ability to grow at least 10, 20, 30, 40, or 50% more rapidly, or at least 10, 20, 30, 40, or 50% longer, in the presence of a selected concentration of NH₃ or NH₄⁺ than can a naturally occurring N. eutropha.

Administered “in combination,” as used herein, means that two (or more) different treatments are delivered to the subject during the course of the subject's affliction with the disorder, e.g., the two or more treatments are delivered after the subject has been diagnosed with the disorder and before the disorder has been cured or eliminated. In some embodiments, the delivery of one treatment is still occurring when the delivery of the second begins, so that there is overlap. This is sometimes referred to herein as “simultaneous” or “concomitant” or “concurrent delivery”. In other embodiments, the delivery of one treatment ends before the delivery of the other treatment begins. This is sometimes referred to herein as “successive” or “sequential delivery” or “consecutive delivery.” In embodiments of either case, the treatment is more effective because of combined administration. For example, the second treatment is a more effective, e.g., an equivalent effect is seen with less of the second treatment, or the second treatment reduces symptoms to a greater extent, than would be seen if the second treatment were administered in the absence of the first treatment, or the analogous situation is seen with the first treatment. In some embodiments, delivery is such that the reduction in a symptom, or other parameter related to the disorder is greater than what would be observed with one treatment delivered in the absence of the other. The effect of the two treatments can be partially additive, wholly additive, or greater than additive (i.e., synergistic). The delivery can be such that an effect of the first treatment delivered is still detectable when the second is delivered.

Administered “in combination,” as used herein, means that two (or more) different preparations, e.g., consumer product, e.g., finished consumer product, e.g., ammonia oxidizing containing product, are delivered to the subject over a given period of time. The two or more different preparations may be delivered simultaneously, or successively.

A “natural product” is or may comprise a product that may be at least partially derived from nature. It may be anything or comprise anything produced by a living organism, and may include organisms themselves. Natural products may include or comprise an entire organism, and part of an organism (e.g., a leaf of a plant), an extract from an organism, an organic compound from an organism, a purified organic compound from an organism. Natural products may be or comprise organic substances found and cells, including primary metabolites (amino acids, carbohydrates, and nucleic acids) and secondary metabolites (organic compounds found in a limited range of species, e.g., polyketides, fatty acids, terpenoids, steroids, phenylpropanoids, alkaloids, specialized amino acids and peptides, specialized carbohydrates). Natural products may be or comprise polymeric organic materials such as cellulose, lignin, and proteins.

Natural products may be or comprise products for commercial purposes, and may refer to cosmetics, dietary supplements, and foods produced from natural sources. Natural products may have pharmacological or biological activity that may be of therapeutic benefit, e.g., in treating disease or conditions. Natural products may be included in traditional medicines, treatments for cosmetological purposes, and spa treatments. A natural product referred to herein may comprise any one or more of the components described as a natural product to be incorporated into a preparation or formulation comprising one or more other components, e.g., excipients. The preparation or formulation referred to as a natural product may comprise a natural product defined herein and one or more additional components or ingredients. Any of the compositions, preparations, or formulations discussed throughout this disclosure may be or comprise one or more natural products.

As used herein, “presence” or “level” may refer to a qualitative or quantitative amount of a component, e.g., any one or more of an ammonia oxidizing bacteria, ammonia, ammonium ions, urea, nitrite, or nitric oxide. The presence or level may include a zero value or a lack of presence of a component.

The terms “polypeptide”, “peptide” and “protein” (if single chain) are used interchangeably herein to refer to amino acid polymers. The polymer may be linear or branched, it may comprise modified amino acids, and it may be interrupted by non-amino acids. The terms also encompass an amino acid polymer that has been modified; for example, disulfide bond formation, glycosylation, lipidation, acetylation, phosphorylation, or any other manipulation, such as conjugation with a labeling component. The polypeptide can be isolated from natural sources, can be a produced by recombinant techniques from a eukaryotic or prokaryotic host, or can be a product of synthetic procedures.

As used herein, a “subject” may include an animal, a mammal, a human, a non-human animal, a livestock animal, or a companion animal. The term “subject” is intended to include human and non-human animals, for example, vertebrates, large animals, and primates. In certain embodiments, the subject is a mammalian subject, and in particular embodiments, the subject is a human subject. Although applications with humans are clearly foreseen, veterinary applications, for example, with non-human animals, are also envisaged herein. The term “non-human animals” of the disclosure includes all vertebrates, for example, non-mammals (such as birds, for example, chickens; amphibians; reptiles) and mammals, such as non-human primates, domesticated, and agriculturally useful animals, for example, sheep, dog, cat, cow, pig, rat, among others. The term “non-human animals” includes companion animals or pets, for example, dog, cat, horse, rabbit, hamster, guinea pig, gerbil, or hedgehog. The term “non-human animals” includes research animals, for example, mouse, rat, rabbit, dog, cat, pig, among others.

As used herein, the term “surfactant”, includes anionic, cationic, non-ionic, and amphoteric compounds that may lower the surface tension, or interfacial tension, between two liquids or between a liquid and a solid. Surfactants may act as detergents, wetting agents, emulsifiers, foaming agents, and dispersants. Surfactants may be referred to as excipients. Surfactants may include one or more of the following, alone, or in combination with those listed, or other surfactants or surfactant-like compounds, and other compounds described throughout this disclosure: cocamidopropyl betaine (ColaTeric COAB), polyethylene sorbitol ester (e.g., Tween 80 and Tween 20), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr. Bronner's baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K) and combinations thereof. Dr. Bronner's Castile soap and baby soap comprises water, organic coconut oil, potassium hydroxide, organic olive oil, organic fair deal hemp oil, organic jojoba oil, citric acid, and tocopherol.

Surfactants may include Sodium Laurylglucosides Hydroxypropylsulfonate (Suga®nate 160NC), lauramidopropyl betaine (Cola®Teric LMB); Cocamidopropyl hydroxysultaine (Cola®Teric CBS); disodium cocoamphodiacetate (Cola®Teric CDCX-LV); sodium laurylglucosides hydroxypropyl phosphate (Suga®Fax D12).

Surfactants may include sodium lauroyl methyl isethionate (Iselux® LQ-CLR-SB); sodium methyl cocoyl taurate (Pureact WS Conc.); Aqua (and) Sodium Lauroyl Methyl Isethionate (and) Cocamidopropyl Betaine (and) Sodium Cocoyl Isethionate (and) Sodium Methyl Oleoyl Taurate (Iselux®SFS-SB).

As used herein, “transgenic” means comprising one or more exogenous portions of DNA. The exogenous DNA is derived from another organism, e.g., another bacterium, a bacteriophage, an animal, or a plant.

“Finished consumer product” as that term is used herein, refers to a consumer product that is ready and/or suitable for release into commerce. For example, a finished consumer product may be a product, e.g., a consumer product, that contains one or more components, and has been prepared and packages for use by an end-user, e.g., a consumer. In an embodiment a finished consumer product is disposed in the end-use container in which it will be used by the end-user. In an embodiment, a finished consumer product may be one or more of manufactured, mixed, integrated, disposed in an end-use container, sterilized, tested, and sealed, in any particular order.

“Finished cosmetic product” as that term is used herein, refers to a cosmetic product that is ready and/or suitable for release into commerce. For example, a finished cosmetic product may be a product, e.g., a cosmetic product, that contains one or more components, and has been prepared and packages for use by an end-user, e.g., a consumer. In an embodiment a finished cosmetic product is disposed in the end-use container in which it will be used by the end-user. In an embodiment, a finished cosmetic product may be one or more of manufactured, mixed, disposed in an end-use container, sterilized, tested, and sealed, in any particular order.

A “unit” of a finished consumer product refers to a single entity of finished consumer product that may form an individual or complete component for use by end-user or for sale. In some embodiments a unit may be a single entity but may form an individual component of a larger or more complex whole. In an embodiment, a unit may be an individual end-use container that contains a consumer product, for sale or for use by an end-user.

“End-user”, as that term is used herein, refers to a person who will use a finished consumer product, e.g., by applying the finished consumer product to a surface or to himself or herself or which applies, or provides, the finished consumer product, to a subject, e.g., another person, or an animal, e.g., a companion animal.

“Recommended life”, as that term is used herein, refers to a suggested period of time that may be provided by a manufacturer of the finished consumer product. The recommended life may be based on testing performed that establishes that use of the product during the period of time provides for treatment, e.g., providing a health benefit or supporting engraftment of a particular microorganism. The recommended life may be based on testing performed that establishes that use of the product during the period of time provides for no noticeable adverse effects.

“End-use container”, as that term is used herein, refers to a vessel that houses a consumer product, e.g., finished consumer product. The end-use container may allow for delivery of the finished consumer product from the vessel to the outside environment. In certain embodiments, the end-use container may prevent or reduce retrograde flow of the contents of the container. The end-use container may be configured to provide one-way flow and/or zero-dead volume.

The end-use container may be comprised of any suitable material that is compatible with the contents of the container and the external environment. For example, the end-use container may be made of glass, aluminum, or one or more polymers, for example, a high density polyethylene polymer.

The end-use container may comprise a delivery system, e.g., a system or mechanism that may allow contents from the container to be disposed outside of the container. For example, the delivery system may comprise a dispenser, for example, utilizing a pump.

“Microbiome” refers to a population, e.g, one or more microorganisms that live on a surface of a subject, e.g., in the gut, mouth, skin, and/or elsewhere in a subject. The population may have one or more beneficial functions and/or benefits, relevant to supporting the life of a subject.

“Preservative”, as that term is used herein, refers to a compound that kills or inhibits the growth of a microbe, e.g., a bacterium or fungus. Exemplary preservatives include those listed in Annex VI at the end of the Detailed Description, herein. Preservatives referred to herein may be referred to as anti-bacterial preservatives. Preservatives referred to herein may not be referring to anti-oxidant preservatives. Preservatives referred to herein may not be referring to anti-viral preservatives. In some embodiments, preservatives referred to herein may not be referring to anti-fungal preservatives.

As used herein, “Sterility Assurance Level” is the probability of a single unit, for example, a single consumer product, e.g., single finished consumer product being non-sterile after it has been subjected to sterilization. Sterility Assurance Level (SAL) may be about 10⁻⁵ or as 10⁻⁶, which is means a 1 in 100,000 chance (for 10⁻⁵) or a 1 in 1,000,000 chance (for 10⁻⁶) of a non-sterile unit. SAL may also describe the killing efficacy of a sterilization process. A very effective sterilization process has a very low SAL, for example, 10⁻⁵, or 10⁻⁶.

As used herein, “treatment of a disease or condition” refers to reducing the severity or frequency of at least one symptom of that disease or condition, compared to a similar but untreated patient. Treatment can also refer to halting, slowing, or reversing the progression of a disease or condition, compared to a similar but untreated patient. Treatment may comprise addressing the root cause of the disease and/or one or more symptoms.

As used herein a “therapeutically effective amount” refers to a dose sufficient to prevent advancement, or to cause regression of a disease or condition, or which is capable of relieving a symptom of a disease or condition, or which is capable of achieving a desired result. A therapeutically effective dose can be measured, for example, as a number of bacteria or number of viable bacteria (e.g., in CFUs) or a mass of bacteria (e.g., in milligrams, grams, or kilograms), or a volume of bacteria (e.g., in mm³).

As used herein, the term “viability” may refer to a microorganism's ability to metabolize compounds, e.g., harmful compounds. “Viability” may refer to a microorganism's ability to generate a byproduct, e.g., a beneficial byproduct.

“Viability” may refer to an ammonia oxidizing microorganism's ability to oxidize ammonia, ammonium, or urea to nitrite at a pre-determined rate. In some embodiments, the rate refers to the conversion of ammonium ions (NH₄⁺)(e.g., at about 200 mM) to nitrite (NO₂⁻) at a rate of at least 50, 75, 125, or 150 micromoles NO₂⁻ per minute, e.g., about 100-150, 75-175, 75-125, 100-125, 125-150, or 125-175 micromoles/minute, e.g., about 125 micromoles NO₂⁻ per minute.

As used herein, “activation” may refer to an action that may place a microorganism in a more active state. Activation may relate to stimulation of the microorganism to assist in the metabolism of a compound or production of a byproduct. Activation may relate to helping establish a colony or community of microorganisms. Activation may relate to providing an environment that may favor sustainability and/or growth of microorganisms. Activation may relate to accelerating availability of the microorganisms. Activation may take place through the use of an activator.

“Activation,” as used herein, may be used relative to autotrophic microorganisms, e.g., ammonia oxidizing microorganisms. Activation refers to any action that may place the ammonia oxidizing microorganisms in a potentially more active state, e.g., a growth state. Activation may relate to stimulation of autotrophic microorganisms, e.g., ammonia oxidizing microorganisms, to assist in some way in the conversion of at least one of ammonia, ammonium ions, and urea into nitrite, nitric oxide, or nitric oxide precursors. Activation may relate to helping establish a bacterial colony, e.g., to allow for the autotrophic bacteria, e.g., ammonia oxidizing bacteria, to compete with other existing bacteria. Activation may relate to providing an environment that may favor sustainability and/or growth of autotrophic microorganisms, e.g., ammonia oxidizing microorganisms. Activation may relate to accelerating availability of the autotrophic microorganisms, e.g., ammonia oxidizing microorganisms to an environment or a surface. “Activation” may provide for ammonia oxidizing microorganisms to be in an “activated” or “growth state.” “Activation” may take place with the use of an activator.

The microorganisms may come into contact with the activator to provide bacteria microorganism in an “activated” or “growth” state. This may occur within or outside of a container, e.g., end-use container, delivery device, or delivery system, e.g., within a first chamber, a second chamber, a mixing chamber, a third or additional chamber, or combinations thereof.

The activator may be in a solution, suspension, a powder, e.g., crystalline form, a media, a buffer, or disposed in or provide as a suitable carrier for maintaining the activator. The activator may be present within the container, e.g., end-use container, or may be present separately from the container, e.g., in another container.

The microorganisms may be in any suitable form for maintaining the microorganisms in a desired state, e.g., a storage state, e.g., an aqueous suspension, gel, or powder form. A time-release, or controlled release activator may be used as an activator.

The activator may be at least one of ammonia, ammonium ions, or urea. The activator may be an ammonium salt, e.g., ammonium chloride or ammonium sulfate. The concentration of the activator, e.g., ammonium salt, e.g., ammonium chloride or ammonium sulfate may be in a range of about 10 micromolar to about 100 millimolar. In certain aspects the concentration of the activator, e.g., ammonium salt, e.g., ammonium chloride or ammonium sulfate may be in a range of about 0.5 mM to about 50 mM. The at least one of ammonia, ammonium ions, or urea may be in a medium or a buffer to promote growth of ammonia oxidizing microorganisms, e.g., an AOM media or a growth media.

“Actuation,” as used herein, means that some action is being taken, e.g., a process is being started or something is being put into motion. In some embodiments, actuation may refer to the breaking of a barrier of a container, e.g., end-use container, mixing of the contents of the container, or the initiation of movement of one or more contents of a container, e.g., delivery of one or more contents of the container to outside of the container, e.g., to a surface or an environment. In some embodiments, actuation of the barrier may comprise one or more materials degrading over time that will allow contact of contents of the first chamber and the second chamber, e.g., a controlled release of contents of the first chamber, or a controlled release of contents from the second chamber, or both.

Actuation may also mean some action that allows delivery of contents of the container, e.g., end-use container to outside of the container, e.g., to a surface or an environment. In an embodiment, if actuation of an end-use container containing a finished consumer product occurs, a seal of a container may be broken in order to deliver the finished consumer product outside of the container. In an embodiment, if actuation of an end-use container containing a finished consumer product occurs, a container may be opened, e.g., a valve may be opened, or pump may be triggered in order to deliver the finished consumer product outside of the container.

A “barrier,” as used herein, may mean any structure or configuration that may serve to obstruct passage or to maintain separation of the contents of the container, e.g, a finished consumer product, e.g., between a first chamber and a second chamber of a container. The barrier may be in the form of a valve, e.g., a check valve, filtering material, film, wax, lipid, polymer, or controlled release material, e.g., slow release material. The barrier may be a material or a structure that upon actuation of a container, it may allow passage of contents from the container to the outside of the container. The barrier may be a material or a structure that upon actuation of the container, it may allow passage of contents from a first chamber into a second chamber, passage of contents from a second chamber into a first chamber, or both. The barrier may be disrupted upon actuation, e.g., through piercing, puncturing, stabbing, perforating, penetrating, splitting, twisting, opening or tearing the barrier. The barrier may be in a form of a valve, e.g., a check valve, a flexible or inflexible material that may not degrade upon contact with one or more contents of the container, or a flexible or inflexible material that may degrade upon contact with one or more contents of the container, a filter material. The barrier may be made of any material suitable for its purpose, e.g., a material that may serve to obstruct passage or to maintain separation, e.g., a polymeric material or metal material.

The barrier may mean any structure that may provide for sealing of the container, e.g., sealing of the container so as to not allow the contents of the container, e.g, a finished consumer product, to be exposed to the environment outside of the container, and to not allow anything in the environment outside of the container to enter the interior of the container, e.g, the finished consumer product.

In some embodiments, the states most relevant to the present disclosure are the state of growth, e.g., maximal growth, characterized by a pH of at least about 7.6, ammonia, trace minerals, oxygen and carbon dioxide. Another state may be characterized by a pH of about 7.4 or less and characterized by an absence of carbon dioxide. Under low carbon dioxide conditions, ammonia oxidizing microorganisms, e.g., Nitrosomonas, continues to oxidize ammonia into nitrite and generates ATP, but lacking carbon dioxide, e.g., lacking sufficient carbon dioxide, to fix and generate protein, it instead generates polyphosphate, which it uses as an energy storage medium. This may allow the ammonia oxidizing microorganisms to remain in a “storage state” for a period of time, e.g., a pre-determined period of time, for example, at least 1, 2, 3, 4, 5, 6, 7, days, 1, 2, 3, 4 weeks, 1, 2, 3, 4, 5, 6, 7, 8, 9, 10, 11, 12 months, 1, 2, 3, 4, or 5 years. In some embodiments, the ammonia oxidizing microorganisms may remain in a storage state for at least about 6 months to about 1 year.

As used herein, “growth state” refers to autotrophic bacteria, e.g., ammonia oxidizing bacteria, in a state or in an environment, e.g., a media, e.g., a culture media, e.g., a growth media, that may have a pH of at least about 7.6. Levels of at least one of ammonia, ammonium ions, and urea may be between about 1 micromolar and 1000 millimolar. Levels of trace materials are between about 0.01 micromolar iron and 200 micromolar iron. Levels of oxygen are between about 5% and 100% oxygen saturation (e.g., of media). Levels of carbon dioxide are between about 20 ppm and 10% saturation (e.g., of media). In certain aspects, levels of at least one of ammonia, ammonium ions, and urea may be between about 10 micromolar and 100 millimolar. Levels of trace materials are between about 0.1 micromolar iron and 20 micromolar iron. Levels of oxygen are between about 5% and 100% oxygen saturation. Levels of carbon dioxide are between about 200 ppm and 5% saturation (e.g., of media).

As used herein, “polyphosphate loading state” refers to autotrophic bacteria, e.g., ammonia oxidizing bacteria, in a state or in an environment, e.g., a media, e.g., a culture media, e.g., a growth media, that may have a pH of about 7.4, or less. Levels of at least one of ammonia, ammonium ions, and urea are between about 1 micromolar and 2000 millimolar. Levels of trace materials are between 0.01 micromolar iron and 200 micromolar iron. Levels of oxygen are between about 0% and 100% O₂ saturation (e.g., of media). Levels of carbon dioxide are between/less than about zero and 400 ppm, and phosphate levels greater than about 1 micromolar. In certain aspects, levels of at least one of ammonia, ammonium ions, and urea are between about 10 micromolar and 200 millimolar. Levels of trace materials are between 0.1 micromolar iron and 20 micromolar iron. Levels of oxygen are between about 5% and 100% O₂ saturation. Levels of carbon dioxide are between/less than about zero and 200 ppm, and phosphate levels greater than about 10 micromolar.

A purpose of the polyphosphate loading state may be to provide AOM with sufficient ammonia, ammonium ions, and/or urea, and O₂ such that ATP can be produced, but to deny them CO₂ and carbonate such that they are unable to use that ATP to fix CO₂ and instead use that ATP to generate polyphosphate which may be stored by the bacteria.

As used herein, the term “storage state” refers to autotrophic bacteria, e.g., ammonia oxidizing bacteria, in a state or in an environment, e.g., a media, e.g., a culture media, e.g., a growth media, having a pH of about 7.4 or less (in some embodiments, the pH may be 7.6 or less). Levels of at least one of ammonia, ammonium ions, and urea are between about 1 and 1000 micromolar. Levels of trace materials are between about 0.1 and 100 micromolar. Levels of oxygen are between about 0 and 100% saturation (e.g., of media). Levels of carbon dioxide are between about 0 and 800 ppm. In certain aspects, levels of at least one of ammonia, ammonium ions, and urea are between about 10 and 100 micromolar. Levels of trace materials are between about 1 and 10 micromolar. Levels of oxygen are between about 0 and 100% saturation (e.g., of media). Levels of carbon dioxide are between about 0 and 400 ppm.

Ammonia oxidizing microorganisms (AOM) are produced according to some embodiments of the present disclosure by generating AOM biomass during a growth state, then exposing the AOM to a polyphosphate loading state and then removing the media and resuspending the AOM in a buffer, e.g., a storage buffer (i.e., the storage state).

“Growth media” or “AOM media,” as referred to herein may comprise the following components of Table 1 or Table 2:

TABLE 1

| | Weight/ | | Final | |
|---|---|---|---|---|
| | Volume | | Concentration | |
| | (in ~1.5 L) | | (in ~1.5 L) | |
| (NH₄)₂SO₄ (MW 132.14) | 4.95 | g | 50 | mM NH₄⁺ |
| KH₂PO₄ (MW 136.1) | 0.616 | g | 3.0 | mM |
| 1M MgSO₄ 7H₂O | 1.137 | ml | 0.76 | mM |
| 1M CaCl₂ 2H₂O | 0.3 | ml | 0.2 | mM |
| 30 mM FeCl₃/50 mM EDTA | 0.5 | ml | 10 μM/16.7 μM | |
| 50 mM CuSO₄ 5H₂O | 30 | μl | 1.0 | μM |

Add 1400 ml ddH₂O to flask. Autoclave (or filter-sterilize).
Store at room temperature.

| After autoclaving add: | | | |
|---|---|---|---|
| Phosphate Buffer (pH 8) | 100 | ml | 32 mM KH₂PO₄/ |
| | | | 2.7 mM NaH₂PO₄•H₂O |
| 5% Na₂CO₃ | 12 | ml | 0.04% |
| (expected pH is about 8.0) | | | |
| Store at room temperature | | | |

TABLE 2

| | Batch medium | | Feeding solution | |
|---|---|---|---|---|
| | Weight/Volume | | Weight/Volume | |
| | (1 L) | | (1 L) | |
| | (Final concentration) | | (Final concentration) | |
| (NH₄)2SO₄ (MW 132.14) | 3.3 g | (50 mM NH₄⁺) | 13.2 g | (200 mM NH₄⁺) |
| KH₂PO₄ (MW 136.1) | 1.23 g | (9.0 mM) | 0.41 g | (3.0 mM) |
| 1M MgSO₄ | 0.758 ml | (0.76 mM) | 0.758 ml | (0.76 mM) |
| 1M CaCl₂ | 0.2 ml | (0.2 mM) | 0.2 ml | (0.2 mM) |
| 30 mM FeCl₃/50 mM EDTA | 0.333 ml | (10 μM/16.7 μM) | 0.333 ml | (10 μM/16.7 μM) |
| 50 mM CuSO₄ | 20 μl | (1.0 μM) | 20 μl | (1.0 μM) |

| | | |
|---|---|---|
| ddH₂O | 1000 ml | 1000 ml |

Autoclave each solution and store at room temperature.

“Deterioration-based expiration date,” as used in here, is a date after which a product, e.g. a consumer product, e.g, a finished consumer product, is expected to degrade in some way that would make it unsuitable for its intended purpose. In some instances it may be a date after which a product, e.g., a consumer product, e.g., a finished consumer product has degraded. An indication that the product has degraded may be provided by way of an indicator located on or in the consumer product or consumer product packaging, e.g, end-use container that provides for some sign that the product has degraded. For example, this may be accomplished by way of a color indicator.

“Biome-compatible-based expiration date,” as used here, is a date after which a product, e.g. a consumer product, e.g, a finished consumer product, is expected to become contaminated in some way that would make it unsuitable for its intended purpose. In some instances it may be a date after which a product, e.g., a consumer product, e.g., a finished consumer product has become contaminated. An indication that the product has become contaminated may be provided by way of an indicator located on or in the consumer product or consumer product packaging, e.g, end-use container that provides for some sign that the product has been contaminated. For example, this may be accomplished by way of a color indicator. A contaminant may refer to something that may make the product unsuitable for its intended use, and may include any item that is not in the finished consumer product at the time of sealing the product and/or subsequent to sterilization of the product.

“Biome-friendly” refers to something, e.g, a product, e.g., a consumer product, e.g., a finished consumer product that may allow for minimal disruption of a microbiome of a subject. For example, biome-friendly refers to a product that may be applied to a subject that may allow the microbiome at the point of application to be maintained, minimally disrupted, and/or able to return to the microbiome after a period of time after application of the product. In embodiments, biome-friendly may refer to ammonia oxidizing bacteria-friendly, in that the product may allow for minimal disruption of the ammonia oxidizing bacteria of a subject.

“Biome-friendly” may refer to a certification which is applied to any test ingredient or product that has passed the laboratory testing standard and determined to have Minimal Antimicrobial Activity (MAA). MAA is an in vitro laboratory test quantifying the reduction in cellular metabolic activity and cellular viability using a test organism as compared to control testing under conditions appropriate to the use pattern for the specific ingredient or product. Ingredients or products achieving a quantitative result consistent with a minimal reduction in test organism metabolic activity (for example, less than 50%, less than 25%, or less than 10%) and cellular viability under test conditions may be certified as having minimal broad-spectrum antimicrobial activity on a mammalian microbiome.

In embodiments, “biome-friendly” may be referred to as “biome-compatible.”

Throughout this disclosure, formulation may refer to a composition or preparation.

TABLE 3
Potential components of a biome-friendly product.

| Component | Category |
|---|---|
| Decyl glucoside | Surfactant/Cleanser |
| cocamidopropyl betaine (ColaTeric COAB) | Surfactant/Cleanser |
| polyethylene sorbitol ester (e.g., Tween 80) | Surfactant/Cleanser |
| ethoxylated lauryl alcohol (RhodaSurf 6 NAT) | Surfactant/Cleanser |
| sodium laureth sulfate/lauryl glucoside/ | Surfactant/Cleanser |
| cocamidopropyl betaine (Plantapon 611 L UP) | |
| sodium laureth sulfate | Surfactant/Cleanser |
| (e.g., RhodaPex ESB 70 NAT) | |
| alkyl polyglucoside (e.g., Plantaren 2000 N UP) | Surfactant/Cleanser |
| sodium laureth sulfate (Plantaren 200) | Surfactant/Cleanser |
| Dr. Bronner's Castile soap | Surfactant/Cleanser |
| Dr. Bronner's baby soap | Surfactant/Cleanser |
| Lauramine oxide (ColaLux Lo) | Surfactant/Cleanser |
| sodium dodecyl sulfate (SDS) | Surfactant/Cleanser |
| polysulfonate alkyl polyglucoside | Surfactant/Cleanser |
| (PolySufanate 160 P) | |
| sodium lauryl sulfate (Stepanol-WA Extra K) | Surfactant/Cleanser |
| Sodium Laurylglucosides Hydroxypropylsulfonate | Surfactant/Cleanser |
| (Suga ®nate 160NC) | (bio-based) |
| lauramidopropyl betaine (Cola ®Teric LMB) | Surfactant/Cleanser |
| Cocamidopropyl hydroxysultaine | Surfactant/Cleanser |
| (Cola ®Teric CBS) | |
| disodium cocoamphodiacetate | Surfactant/Cleanser |
| (Cola ®Teric CDCX-LV) | |
| sodium laurylglucosides hydroxypropyl phosphate | Surfactant/Cleanser |
| (Suga ®Fax D12). | |
| sodium lauroyl methyl isethionate | Surfactant/Cleanser |
| (Iselux ® LQ-CLR-SB) | |
| sodium methyl cocoyl taurate (Pureact WS Conc.) | Surfactant/Cleanser |
| Aqua (and) Sodium Lauroyl Methyl Isethionate | Surfactant/Cleanser |
| (and) Cocamidopropyl Betaine (and) | |
| Sodium Cocoyl Isethionate (and) | |
| Sodium Methyl Oleoyl Taurate (Iselux ®SFS-SB) | |
| Coco glucoside (Plantacare 818) | Surfactant/Cleanser |
| | (bio-based) |
| Sodium cocoyl-glycinate | Surfactant/Cleanser |
| | (bio-based) |
| Caprylic/Capric triglyceride (Myritol) | Conditioner |
| Cationic guar (N-Hance) | Conditioning/ |
| | de-tangling |
| Coconut oil | Conditioning |
| Apple saccharides, e.g., pyrus | Humectant |
| malus (apple) fruit extract | |
| e.g., pyrus malus (apple) fruit extract and glycerin | |
| (Botanimoist AMS); | |
| Hydrolyzed quinoa | Moisture binding |
| Hydrolyzed adansonia digitata (Baobab) | Softener |
| Seed Protein | |
| Hydroxyethylcellulose (Natrosol) | Viscosity modifier |
| Hydroxypropylcellulose (Klucel MCS) | Viscosity modifier/ |
| | film former |
| Hydrolyzed soy protein (Soy Tein NPNF) | |
| Natural Rose Hydrosol | Fragrance |
| Natural Rose Water | Fragrance |
| Damascena flower water | Fragrance |
| Coco-glucoside and glyceryl oleate | Lipid layer |
| (Lamisoft ® PO 65) | enhancer |
| Polysorbate 80 | Emulsifier |
| Citric acid | pH stabilizer |
| Squalene | Moisturizer |
| Water (i.e. deionized water) | |

### 2. Consumer Products

Consumer products are provided that may be biome-friendly or biome-compatible. The consumer products may comprise one or more components that may be biome-friendly.

The consumer products may generally be supportive of a microbiome of a subject. The consumer product may, in the absence of treatment, e.g., sterilization or addition of a preservative, support microbe growth, e.g., bacterial or fungal growth, e.g., as measured by U.S.P. 51, Antimicrobial Effectiveness Testing or U.S.P. 62, Microbial Examination of Nonsterile Products. In some embodiments, the consumer product will support growth of a microbe, e.g., a bacterium or fungus, if exposed to challenge with the microbe. The consumer product may allow the microbiome of the subject to be maintained.

The consumer products disclosed herein may be substantially free of microorganisms. For instance, the consumer products may be substantially free of ammonia oxidizing microorganisms (AOM). The consumer products disclosed herein may comprise one or more microorganisms, e.g., beneficial microorganisms, e.g., non-pathogenic bacteria. The consumer products disclosed herein may comprise ammonia oxidizing microorganisms.

As disclosed herein, a “consumer product” may refer generally to any article, or component part thereof, produced for or distributed to a consumer. The consumer product may be configured for use in or around the household or residence, school, in recreation, or for personal use, consumption or enjoyment of a consumer. A consumer product may include any product classified as a consumer product by the U.S. Consumer Product Safety Commission or in the Consumer Product Safety Act. A consumer product may include any component or ingredient intended to be included in a consumer product as defined by the U.S. Consumer Product Safety Commission or in the Consumer Product Safety Act.

In accordance with certain embodiments, the consumer product does not include a cosmetic product. The consumer product may be any product not intended to be rubbed, poured, sprinkled, or sprayed on, introduced into, or otherwise applied to the human body for cleansing, beautifying, promoting attractiveness, or altering the appearance. The consumer product may be substantially free of a cosmetic product. The consumer product may not be and/or not comprise a cosmetic product selected from a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

The consumer products of the disclosure may be, or include, or be disposed in any one or more of household products, baby products, pet products, food/beverage products, and personal products.

The consumer products of the disclosure may be, or include, or be disposed in a household product. The household product may be packaged or formulated as a surface conditioner. As disclosed herein, a surface conditioner may be or comprise a substance formulated to alter the condition of the surface that it's applied to.

The household products may be, or include, or be disposed in any one or more of an air freshener, air conditioning product, e.g. anti-mold air conditioning product, surface conditioner, deodorizer, surface cleaner, glass cleaner, bathroom cleaner, e.g., toilet or shower cleaner, appliance, vacuum, storage or organization device, home improvement device, pest control product, dinnerware, serveware, glassware, drinkware, cookware, bakeware, utensil, cutlery, kitchen tool, kitchen cleaner, furniture, furniture cleaner, floor or carpet cleaner, disinfectant, soap, trash bag, grocery, food storage bag or container, cooler, water bottle, disposable tableware, dishwashing product, car cleaning or detailing product, towel, linen, window treatment, media, e.g. a book, music or movie, sport or outdoor recreational equipment, exercise or fitness equipment, sponge, lamp, light, rug, luggage, scrubber, dusting product, lawn, garden, patio, outdoor cooking, footwear conditioning product, cotton product, e.g. cotton bud, or paper product, e.g. toilet paper, facial tissue, or paper towel.

The household product may be a device or item. The device or item may be or comprise one of more of the household products described herein. The household product may be formulated as a solid, liquid, gas, or gel product. In some embodiments, the household product may comprise or be contained in a disposable sponge or wipes.

The household product may be or include sport equipment, outdoor recreational equipment, exercise, or fitness equipment. For example, the household product may be or comprise a yoga mat, gym mat or flooring, gymnastics or wrestling mat, towel, sleeping bag, tent, sports bag or other carrying equipment, climbing equipment, cycling equipment, water sports equipment, snow sports equipment, weights, weight-training machines, cardiovascular exercise machines, and other sports-related equipment. The household product may be or include a sport, recreational, exercise, or fitness equipment cleaner, polisher, or conditioner.

The consumer product may be, or include, or be disposed in a laundry product. For example, the consumer product may be or comprise a laundry detergent, bleach, dryer sheet, scent, stain remover, post-laundry finishing spray, or fabric softener. The consumer product may be, or include, or be disposed in a textile or fabric. The consumer product may comprise a textile or fabric having or containing the consumer product, for example, a textile or fabric to which the consumer product has been applied.

The consumer products of the disclosure may be, or include, or be disposed in a baby product. The baby product may be or include a car seat, stroller, carrier, nursery, diapering, nursing, bathroom, toy, training, or infant safety product or accessory. The baby product may be itself biome-friendly, may comprise one or more biome-friendly components, e.g., textiles or surfaces, or may be treated to be biome-friendly. In at least some embodiments, the baby product may be a bottle, baby bathing product, pacifier, teething product, crib, bassinet, swing, utensil, cup, plate, storage, clothing or diaper product.

The consumer products of the disclosure may be, or include, or be disposed in a pet product. The pet product may be or include a pet health, safety, training, cleaning, grooming, bathing, pest management, crate carrier, bowl, collar, harness, leash, bed, accessory, litterbox, litter, diaper pad, toy, storage container, or treat product. The pet product may be a pet dietary supplement. The pet product may be itself biome-friendly, may comprise one or more biome-friendly components, e.g., textiles or surfaces, or may be treated to be biome-friendly.

The consumer products of the disclosure may be, or include, or be disposed in a food or beverage product. The food or beverage product may be or include a dietary or nutritional supplement, i.e. a vitamin, mineral, specialty (e.g., fish oil, fiber, probiotic, prebiotic, glucosamine, chrondroitin, CoQ10), herbal, botanical, sports, or weight management supplement. The food or beverage product may be packaged in a biome-friendly material, for example, the packaging may render the consumer product biome-friendly. The food or beverage product may be a liquid, e.g., juice or smoothie, solid, e.g., a bar or powder, or gel food or beverage product. The food or beverage product may be formulated to be consumed with water or with other ingredients, for example, as a cooking supplement. The food or beverage product may be a cooking oil.

The consumer products of the disclosure may be, or include, or be disposed in a personal product. The personal product may be or comprise an electronic device and/or a component thereof. The personal product may be or include, for example, a television, video game system, audio system, headphone, mobile phone, wearable device, tablet, e-reader, or technology accessory, i.e., a mobile technology accessory. The electronic device may be or include a computer, e.g., a desktop computer, laptop computer, or mobile computer. The electronic device may comprise one or more biome-friendly components, e.g., screen, keys, mouse or trackpad, remote control, removable case, fixed housing, or a textile, metal, or plastic component.

The personal product may be or comprise an accessory. The personal product may be an accessory, e.g., a fashion accessory. The accessory may be or include, for example, a handbag, wallet, keychain, hat, scarf, belt, glove, mitten, jewelry, hair accessory, glasses, or sunglasses. The accessory may be or include, for example a backpack or luggage item. The accessory may be or comprise one or more biome-friendly components. In at least some embodiments, the accessory is a personal accessory.

The personal product may be or comprise an office or school supply. The office or school supply may be or include, for example, office or school furniture, an electronic device, e.g., calculator, copy machine, printer, or telephone, notebook, notepad, book, pen, pencil, marker, or other writing utensil, eraser, scissors, ruler, tape, glue, sharpener, binder, paper clip, bookmark, flag, whiteboard, blackboard, or projector screen. The office or school supply may be or comprise one or more biome-friendly components.

Cosmetic products, e.g., finished cosmetic products, are provided that may be biome-friendly or biome-compatible. The cosmetic products may comprise one or more components that may be biome-friendly. As disclosed herein, “cosmetic product” may generally refer to an article intended to be rubbed, poured, sprinkled, or sprayed on, introduced into, or otherwise applied to the human body for cleansing, beautifying, promoting attractiveness, or altering the appearance. A cosmetic product may include any product classified as a cosmetic product by the U.S. Food and Drug Administration. A cosmetic product may include any ingredient intended to be included in a cosmetic product as defined by the U.S. Food and Drug Administration.

The cosmetic products of the present disclosure may be, or include, or be disposed in any one or more of a baby product, e.g., a baby shampoo, a baby lotion, a baby oil, a baby powder, a baby cream; a bath preparation, e.g., a bath oil, a tablet, a salt, a bubble bath, a bath capsule; an eye makeup preparation, e.g., an eyebrow pencil, an eyeliner, an eye shadow, an eye lotion, an eye makeup remover, a mascara; a fragrance preparation, e.g., a colognes, a toilet water, a perfume, a powder (dusting and talcum), a sachet; hair preparations, e.g., hair conditioners, hair sprays, hair straighteners, permanent waves, rinses, shampoos, tonics, dressings, hair grooming aids, wave sets; hair coloring preparations, e.g., hair dyes and colors, hair tints, coloring hair rinses, coloring hair shampoos, hair lighteners with color, hair bleaches; makeup preparations, e.g., face powders, foundations, leg and body paints, lipstick, makeup bases, rouges, makeup fixatives; manicuring preparations, e.g., basecoats and undercoats, cuticle softeners, nail creams and lotions, nail extenders, nail polish and enamel, nail polish and enamel removers; oral hygiene products, e.g., dentrifices, mouthwashes and breath fresheners; bath soaps, e.g., foaming body cleansers, and detergents, deodorants, douches, feminine hygiene deodorants; shaving preparations, e.g., aftershave lotions, beard softeners, talcum, preshave lotions, shaving cream, shaving soap; skin care preparations, e.g., cleansing, depilatories, face and neck, body and hand, foot powders and sprays, moisturizing, night preparations, paste masks, skin fresheners; and suntan preparations, e.g., gels, creams, and liquids, and indoor tanning preparations.

A finished consumer product may be provided comprising a cosmetic product, e.g, a shampoo, a body cleanser, a conditioner. The finished consumer product may be disposed in an end-use container.

### 3. Consumer Product Formulations

The finished consumer product may have one or more properties. The consumer product, or finished consumer product may be substantially free of a preservative, e.g., a paraben. The end-use container may be configured to reduce retrograde flow. The consumer product, or finished consumer product may be sterilized. The sterilization may include irradiation, e.g., gamma irradiation, or heat sterilization. The consumer product may be formulated as one of the compositions disclosed herein. The consumer product may comprise, consist essentially of, or consist of a viscosity modifier, one cleanser/surfactant, two cleansers/surfactants, a humectant, a conditioner, e.g., surface or skin conditioner, and a fragrance.

The consumer product may comprise any one or more of the following components: water, ammonia, decyl glucoside, lauryl glucoside, sodium citrate, lauramine oxide, polysorbate 20, glycerin, disodium 2-sulfolaurate, sodium methyl 2-sulfolaureate, tetrasodium glutamate diacetate, sodium sulfate, laureth-7, propanediol, gluconolactone, xanthan gum, propylene glycol, sodium benzoate, sorbitol, calcium chloride, protease, amylase, lipase, mannase, sodium formate, pectase, lyase, or calcium bromide.

The consumer product or finished consumer product may comprise, consist essentially or consist of the following composition(s):

| | | Preferred | |
|---|---|---|---|
| | Preferred | Con- | |
| | Con- | centration | |
| Component | centration | Range | Description |
| Klucel MCS | 1.5% | 1.0-2.0% | Viscosity Modifier |
| (Hydroxypropyl | | | |
| Cellulose) | | | |
| Cocamidopropyl Betaine | 20.0% | 15.0%-30.0% | Cleanser/Surfactant |
| Decyl Glucoside | 7.5% | 0.0-10.0% | Cleanser/Surfactant |
| Botanimoist AMG | 3.0% | 0.0-4.0% | Humectant |
| (Glycerine + Apple extract | | | |
| Hydrolyzed Baobab | 2.0% | 0.0-4.0% | Skin Conditioner |
| Protein | | | |
| Natural Rose Hydrosol | 10.0% | 0.0-15.0% | Fragrance |

This consumer product may be used as a household product, e.g., for a surface conditioner. The product includes water to make 100%. In some embodiments, the consumer product, e.g., household product, may or may not contain citric acid, and the citric acid may be needed in cases where pH stabilization is required or desired.

(ii) The consumer product or finished consumer product may comprise, consist essentially or consist of the following composition(s):

| | | Preferred | |
|---|---|---|---|
| | Preferred | Concentration | |
| Component | Concentration | Range | Description |
| Klucel MCS | 0.25% | 0.10-0.50% | Viscosity |
| (Hydroxypropyl | | | modifier |
| Cellulose) | | | |
| Cocamidopropyl | 8.00% | 5.0-12.0% | Cleanser/ |
| betaine | | | Surfactant |
| Decyl Glucoside | 4.0% | 0.0-6.0% | Cleanser/ |
| | | | Surfactant |
| Botanimoist AMG | 2.0% | 0.0%-4.0% | Humectant |
| (Glycerine + | | | |
| Apple extract) | | | |
| Hydrolyzed | 1.0% | 0.0-3.0% | Skin |
| Quinoa Protein | | | Conditioner |
| conditioner | | | |
| Natural Rose Hydrosol | 7.5% | 0.0%-10.0% | Fragrance |
| Citric Acid | Added as | As needed, | pH stabilizer |
| | needed, usually | usually in very | |
| | in very small | small amounts, | |
| | amounts, for pH | for pH | |
| | stabilization | stabilization | |

This product may be used as a consumer product, e.g., for a cleanser, e.g., for the kitchen, bathroom, or multi-surface cleaner. The product includes water to make 100%. In some embodiments, the consumer product, e.g., cleanser, may or may not contain citric acid, and the citric acid may be needed in cases where pH stabilization is required or desired.

Other hydrolyzed protein may be used, and may include, but is not limited to rice, soy baobab, and oat. Other fragrance alternatives may be contemplated.

The finished consumer product may have one or more, or all the properties described herein.

Other products are contemplated, including personal products, e.g., hair and/or skin conditioners that may comprise, consist essentially of, or consist of the following:

| | Preferred | Preferred | |
|---|---|---|---|
| Component | Concentration | Concentration Range | Description |
| Hydroxypropyl | 1.5% | 0.50-2.50% | Viscosity |
| cellulose (Klucel | | | modifier/ |
| MCS) | | | film former |
| Cationic guar | 0.5% | 0.10-1.5% | Conditioning and |
| (N-Hance) | | | detangling |
| Coconut oil | 1.0% | 0.0-3.0% | Conditioning |
| Fragrance | As desired | As desired | |

The product includes water to make 100%. In some embodiments, the consumer product, e.g., conditioner may or may not contain citric acid, and may be needed in cases where pH stabilization is required or desired.

In accordance with one or more non-limiting embodiments, a consumer product or finished consumer product, for example, a biome-friendly cleaner, may comprise, consist essentially or consist of the following composition(s):

| | | % range |
|---|---|---|
| Component | Sample Ingredient(s) | (example %) |
| Surfactant/ | Sodium laurylglucosides | 50-70% (64.3%) |
| cleanser(s) | hydroxypropylsulfonate | |
| | Lauramidopropyl betaine | 20-40% (28.8%) |
| Fragrance | Rosa Damascena Flower Water | 3-10% (4.6%) |
| Softener | Hydrolyzed Adansonia | 1-5% (1.85%) |
| | Digitata Seed Protein | |
| Moisturizer | Squalene | 0.1-2% (0.45%) |

In accordance with one or more non-limiting embodiments, a consumer product or finished consumer product, for example, a biome-friendly cleaner, may comprise, consist essentially or consist of the following composition(s):

| | | % range |
|---|---|---|
| Component | Sample Ingredient(s) | (example %) |
| | Deionized Water | 70-80% (76.25%) |
| Surfactant/cleanser(s) | Lauramidopropyl betaine | 5-12% (8%) |
| | Decyl Glucoside | 3-7% (4%) |
| Fragrance | Rosa Damascena Flower Water | 4-10% (7.5%) |
| Humectant | Pyrus Malus Fruit | 2-5% (3%) |
| | Extract in glycerin | |
| Moisture binder | Hydrolyzed Quinoa | 0.5-2% (1%) |
| Viscosity | Hydroxypropylcellulose | 0.1-1% (0.25%) |
| modifier/film former | | |

In accordance with various disclosed and non-limiting embodiments, the amount of each individual component in the product may vary by, for example, 1%, 5%, or 10%, while still maintaining similar properties of the product.

The consumer product may be preservative free, e.g., it does not include a preservative. Preservatives may be identified as the compounds listed in Annex VI at the end of the Detailed Description, herein. At most, the finished consumer product may have less than 500 ppb of a preservative, e.g, one or more of those listed in Annex VI. In embodiments, the finished consumer product may have less than 100 ppb of a preservative, e.g., one or more of those listed in Annex VI. A brief list of materials that could be used as “natural” preservatives include: Neem Oil, Lemon juice or oil, Bee Propolis, Rosemary Extract, Grapefruit Seed Extract, Citric acid, Alpha tocopherol (also known as vitamin E), Potassium Sorbate, Phenoxyethanol, Salicylic Acid, Sodium Benzoate, Sorbic Acid, Plant Essential Oils (Thyme, Oregano, Lemongrass, Lavender, Rosemary, and others), Lactic Acid.

At most, the finished consumer product may have between about 1 ppm and 10 ppb of preservative, e.g., one or more of those listed in Annex VI; for example, between about 10 ppb and 50 ppb, for example, between about 50 ppb and 100 ppb, e.g., less than 1000, 900, 800, 700, 600, 500, 400, 300, 200, 100, 50, 40, 10, 5, 1 ppb of one or more preservatives, e.g., one or more preservatives listed in Annex VI.

The consumer product may generally be free of a preservative, e.g., an anti-bacterial preservative. The consumer product may comprise a preservative selected to favor at least one pre-determined species of microorganism. In some embodiments, the consumer product may comprise a preservative formulated to suppress growth or reproduction of at least one pre-determined species of microorganism. The consumer product may comprise a preservative selected to have one or more of anti-oxidant, anti-viral, and anti-fungal properties. Suitable preservatives or preservation methods include reduction of bio-available water, sterilization, and increase of lipid concentration.

In some embodiments, the preservative-free property of the consumer product, e.g., finished consumer product may be evidenced through testing disclosed herein. For example, upon exposure to one or more microorganisms, e.g, microbe, e.g., a bacterium or fungus, e.g., growth of the one or more microorganisms will be supported. For example, the consumer product or finished consumer product, if exposed to challenge with a microbe, e.g., a bacterium or fungus, will support growth of said microbe, e.g., as determined by U.S.P. 51, Antimicrobial Effectiveness Testing (USP31-NF26 Page 67), herein incorporated by reference in its entirety. The consumer product or finished consumer product, in the absence of a treatment, e.g., sterilization treatment or the addition of a preservative, supports microbe growth, e.g., bacterial or fungal growth, e.g., as measured by U.S.P. 51, Antimicrobial Effectiveness Testing.

In some embodiments, the preservative-free property of the consumer product, e.g., finished consumer product may be evidenced by, in the absence of a treatment, e.g., sterilization treatment or the addition of a preservative, supports one or more microorganisms, e.g, microbe, e.g., a bacterium or fungus.

In certain embodiments, the consumer product may be disposed in an end-use container, and the finished consumer product may comprise, consist essentially of, or consist of water, cocamidopropyl betaine, rosa damascena flower water, decyl glucoside, pyrus malus (apple) fruit extract, glycerin, hydrolyzed adansonia digitata (baobab) seed protein, hydroxypropylcellulose. The consumer product may be a surface conditioner or cleaner.

In certain embodiments, the consumer product may be disposed in an end-use container, and the finished consumer product may comprise, consist essentially of, or consist of water, cocamidopropyl betaine, rosa damascena flower water, decyl glucoside, pyrus malus (apple) fruit extract, glycerin, hydrolyzed quinoa, hydroxypropylcellulose, and citric acid. The consumer product may be a surface conditioner or cleaner.

In certain embodiments, the consumer product may be disposed in an end-use container, and the finished consumer product may comprise, consist essentially of or consist of hydroxyethyl cellulose, myritol 313 C8-10 triglycerides, coco-glucoside and glyceryl oleate, polysorbate-80, and natural rose water. The consumer product may be a surface conditioner or cleaner.

The consumer product or finished consumer product may comprise a component added to provide one or more of the following: a fragrance, a color, viscosity, foam forming or foam stability, adhesion, moisture retention, moisture binding, pH stabilization, cleansing, thickening, softening, conditioning, lipid layer enhancing, barrier-forming, or film-forming.

The finished consumer product or the consumer product, may comprise one or more of an antioxidant, fatty substance/oil, thickener, softener, emulsifier, light-screening agent, foam forming and foam stability, antifoaming agent, moisturizer, fragrance, surfactant, filler, sequestering agent, polymers, acidifying or basifying agent, dyes, colorant, pigment, pearlizer, opacifier, organic or inorganic particle, viscosity modifier, cleanser, adherent, moisture binder, pH stabilizer, conditioner, de-tangler, biobased surfactant cleanser, lipid layer enhancer, skin conditioner, and natural hair nutrient such as botanicals, fruit extracts, sugar derivatives and/or amino acids, hydrolyzed proteins, or vitamins.

The consumer product may be formulated or treated to provide a health benefit. The consumer product may be formulated or treated to support a healthy lifestyle. In some embodiments, the consumer product may be formulated to provide a health benefit for a pre-determined anatomical region. For example, the consumer product may be formulated to provide a health benefit to one or more of the skin, scalp, gastrointestinal system, intranasal cavities, and respiratory system of the subject. The consumer product may be formulated to provide a health benefit to one or more of hair, nails, or teeth of the subject. The consumer product may be formulated to provide a health benefit to one or more of the visual system, the auditory system, or the urogenital system. The consumer product may be formulated to mitigate or modify body odor.

The consumer product may be formulated or treated to support engraftment of a beneficial microorganism. In some embodiments, the consumer product may be configured to support engraftment. For example, the consumer product may be constructed or structured to support engraftment of a beneficial microorganism. The consumer product may comprise one or more components having a texture selected to support engraftment of a beneficial microorganism.

### 4. Containers, e.g., End-Use Container, Delivery Devices

In accordance with certain embodiments, a container and/or delivery device may be configured to store and/or deliver any consumer product disclosed herein. The consumer product may be delivered to a site, an environment, or a surface with or without additional components. In certain embodiments, other components may be delivered simultaneously or consecutively, e.g., at least partially before or at least partially after, the delivery of the consumer product commences. In certain embodiments, the container or delivery device may comprise or be referred to as a delivery system.

In some embodiments, the delivery of one component is still occurring when the delivery of the second begins, so that there is overlap. This is sometimes referred to herein as “simultaneous” or “concomitant” or “concurrent delivery.” In other embodiments, the delivery of one component ends before the delivery of the other treatment begins. This is sometimes referred to herein as “successive” or “sequential delivery” or “consecutive delivery.”

A barrier may be provided as part of or within the container to prevent fluid communication between the interior of the container and the exterior environment. The barrier may be in the form of a valve, e.g., check valve, filtering material, film, wax, lipid, polymer, control release material, e.g., a gel, and other materials that may either provide a permanent or temporary barrier between the interior of the container and the exterior environment.

Upon actuation of the container, the barrier may be disrupted to allow disposal of the consumer product from the container to the exterior environment, or a site, an environment, or a surface, to contact the consumer product with a site, an environment, or a surface.

The container may comprise a delivery system. The delivery system may be an applicator or be configured to deliver the contents of the consumer product.

The delivery system may be configured to deliver a consumer product to a surface of an item or a household surface. The consumer product may be in the form of a particle, or a plurality of particles having a particle size to enhance delivery or enhance positioning or contact with a desired target site. The consumer product may be in the form of a liquid, solid, in a suspension or in a solution.

In certain embodiments, the delivery system may comprise a pump to deliver the contents of the interior of the container to a target site, e.g., an environment, e.g., a surface.

In some embodiments, the container may be a single-use container. The container may or may not be pre-loaded (e.g., loaded by a manufacturer or user) with contents, e.g., consumer product, and may be used once by a user, e.g., a consumer or medical professional to deliver the contents of the container to a target site, e.g., an environment, e.g., a surface.

In other embodiments, the container may be a multiple-use container in which the container may or may not be pre-loaded (e.g., loaded by a manufacturer or user) with contents, e.g., consumer product, and may be used once by a user, e.g., a consumer or professional to deliver the contents of the container to a target site, e.g., an environment, e.g., a surface. The container may be re-loaded (e.g., loaded by a manufacturer or user) with contents e.g., ammonia oxidizing microorganisms, and ammonia, ammonium ions and urea, and may be used again by a same or different user, e.g., a consumer or professional to deliver the contents of the container to a target site, e.g., an environment, e.g., a surface.

Pre-loading or re-loading of the contents, e.g., consumer product may comprise a sterilization process to ensure that the contents of the container are sterilized.

In some embodiments, the container may be in the form of a syringe, bottle, ampule, applicator, pouch, e.g., spout pouch, e.g., with screw top. A pump may be attached to the bottle in order to dispense the contents from the container. The container may provide for an aerosol spray or mist. The container may be a squeezable container to allow dispensing of the contents through an opening that is covered by a closure. The container may have a screw type closure, a non-spill closure, a snap cap closure, or a snap flap closure. The container may have an over cap that resides over the dispensing area in which dispensing of the contents occurs. The closure may be fully removable or partially removable, e.g., fully removable form the body of the container, or partially removable and attached by a hinge. The container may be a single use package, e.g., a laminated packet, e.g., that may be torn open to dispense the contents, and disposed after use.

The container, e.g., end-use container may be configured to inhibit retrograde flow, e.g., backflow, e.g., reverse flow, e.g., rearward movement, of material, e.g., the consumer product, into said end-use container. The container, e.g, end use container may be configured to inhibit retrograde flow, e.g., backflow, e.g., reverse flow, e.g., rearward movement, of material, e.g., a contaminant, into said end-use container. The contaminant may be is atmospheric, e.g., an aerosol, or a liquid, e.g., water, or solid, or a gas.

The end-use container may comprise a reservoir in which said consumer product is disposed, and a dispenser through which said consumer product from said reservoir can be dispensed, wherein said dispenser inhibits retrograde flow of material into said reservoir.

The end-use container may comprise a reservoir in which said consumer product is disposed, and a dispenser through which said consumer product from said reservoir can be dispensed, wherein said dispenser inhibits retrograde flow of dispensed consumer product, or atmospheric aerosols, into said reservoir.

The end-use container may be an anti-retrograde flow dispenser comprising a first pressure activated valve disposed in said dispenser and proximal to said reservoir and a second pressure activated valve disposed in said dispenser and distal to said reservoir, wherein the activation pressure of said first valve is higher than the activation pressure of said second valve.

The end-use container may comprise an anti-retrograde mechanism configured to prevent movement of the consumer product in a direction opposite the operational direction associated with dispensing the finished consumer product.

In some embodiments, the container may be substantially free of organisms, e.g., microorganisms. In embodiments the container may be free of other organisms. The container may be sterilized to provide for a container substantially free or free of organisms, e.g., microorganisms.

The consumer product may be provided as a powder, cosmetic, cream, stick, aerosol, salve, wipe, or bandage. The consumer product may be provided as a sponge or wipe. The consumer product may be provided as an oil, e.g., essential oil or cooking oil. The consumer product may be provided as a solid, e.g., solid cleanser (soap), structured surface, e.g., plastic, metal, or wood surface, textile, fabric, or leather. The consumer product may be contained in a packaging.

In some embodiments, the container may comprise an indicator component. The indicator component may a color marker that may develop a color upon the contact of a microorganisms to the interior of the container. The indicator component may be a color marker that may develop a color change upon a change in conditions, e.g., temperature or pH.

The container may be constructed of any material suitable for housing the contents, e.g., a consumer product, e.g., a finished consumer product disclosed herein. For example, the container may be constructed and arranged to be at least partially resistant to at least one of gaseous exchange, water, and light. For example, the container may be constructed of a glass or polymeric material.

The end-use container may be composed of or comprise a polymer, e.g., polyethylene terephthalate (PET), high density polyethylene (HDPE), polypropylene, polycarbonate, polytetrafluoroethylene (Teflon®), polyviylidene fluoride (PVDF), or a cellulosic. The end-use container may be composed of or comprise glass. A sensor, e.g., an oxygen sensor, may be included in the end-use container that may indicate a presence of viable bacterial. The end-use container may allow for passage of at least about 5, 10, 20, 30, 40, 50, 60, 70, 80, 90, 95, 99, or 100 percent of transmission of ionizing radiation, e.g., with gamma rays, e.g., with x-rays, e.g., from an isotope, e.g., cobalt 60, or with ultraviolet, e.g., ultraviolet C (UVC) through the end-use container.

In some embodiments, one or more other organisms, such as ammonia oxidizing microorganisms may be included in the container. An organism of the genus selected from the group consisting of Lactobacillus, Streptococcus, Bifidobacter, and combinations thereof, may be provided in the container. An activator for the ammonia oxidizing bacteria may be provided in the container.

The containers described herein may be adapted to deliver one or more consumer products. The containers described herein may be adapted to deliver one or more therapeutic products.

The weight of the container, delivery system, or delivery device, including or not including the contents of the container may be less than about 50, 100, 200, 300, 400, 500, 600, 700, 800, 900, 1000, 1500, or 2000 grams.

### 5. Dispensing the Consumer Product/Methods of Delivering the Consumer Product

The product may be dispensed in discrete volumes or amounts from the container, e.g., end-use container. The container may dispense the same volume or approximately same volume for each actuation of the container. The product may be dispensed, for example in a discrete volume of between about 0.1 ml and about 5 ml. The discrete volume can be, for example about 0.1 ml, 0.2 ml, 0.25 ml, 0.3 ml, 0.4 ml, 0.5 ml, 0.6 ml, 0.7 ml, 0.75 ml, 0.8 ml, 0.9 ml, 1.0 ml, 1.1 ml, 1.2 ml, 1.4 ml, 1.5 ml, 1.6 ml, 1.8 ml, 2.0 ml, 2.25 ml, 2.5, ml, 2.75 ml, 3 ml, 3.25 ml, 3.5 ml, 3.75 ml, 4.0 ml, 5.0 ml, 6.0 ml, 7.0 ml, 8.0 ml, 9.0 ml, or 10 ml.

The container may dispense the same amount or approximately same amount for each actuation of the container. The product may be dispensed, for example in a discrete amount of between about 0.1 grams (g) and about 5 g. The discrete amount can be, for example about 0.1 g 0.2 g, 0.25 g, 0.3 g, 0.4 g, 0.5 g, 0.6 g, 0.7 g, 0.75 g, 0.8 g, 0.9 g, 1.0 g, 1.1 g, 1.2 g, 1.4 g, 1.5 g, 1.6 g, 1.8 g, 2.0 g, 2.25 g, 2.5, g, 2.75 g, 3 g, 3.25 g, 3.5 g, 3.75 g, 4.0 g, 5.0 g, 6.0 g, 7.0 g, 8.0 g, 9.0 g, or 10 g.

Containers may be configured to dispense a first volume or amount for a first actuation, and a second volume or amount for a second actuation.

The total volume of consumer product in the container may be between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, about 3 and about 5 fluid ounces. In some embodiments, the volume may be about 3.4 fluid ounces.

The container may be constructed to contain between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, or about 3 and about 5 fluid ounces. In some embodiments, the container may be constructed to contain about 3.4 fluid ounces. The container may be a one-chamber container, or any other container disclosed herein.

### 6. Rendering a Consumer Product Biome-Friendly

Methods disclosed herein comprise rendering a consumer product biome-friendly. A consumer product can be rendered biome-friendly by applying a biome-friendly preparation, e.g., formulation or composition, as disclosed herein, to a consumer product. Consumer products which may be rendered biome-friendly by application of a biome-friendly preparation include, for example, household products, laundry products, fabrics or textiles, baby products, pet products, food or beverage products, dietary or nutritional supplements, personal products, electronic devices, accessories, and office or school supplies.

The household products which may be rendered biome-friendly by application of a biome-friendly preparation may be, or include, or be disposed in any one or more of an air freshener, air conditioning product, e.g. anti-mold air conditioning product, surface conditioner, deodorizer, surface cleaner, glass cleaner, bathroom cleaner, e.g., toilet or shower cleaner, appliance, vacuum, storage or organization device, home improvement device, pest control product, dinnerware, serveware, glassware, drinkware, cookware, bakeware, utensil, cutlery, kitchen tool, kitchen cleaner, furniture, furniture cleaner, floor or carpet cleaner, disinfectant, soap, trash bag, grocery, food storage bag or container, cooler, water bottle, disposable tableware, dishwashing product, car cleaning or detailing product, towel, linen, window treatment, media, e.g. a book, music or movie, sport or outdoor recreational equipment, exercise or fitness equipment, sponge, lamp, light, rug, luggage, scrubber, dusting product, lawn, garden, patio, outdoor cooking, footwear conditioning product, cotton product, e.g. cotton bud, or paper product, e.g. toilet paper, facial tissue, or paper towel.

The household product which may be rendered biome-friendly by application of a biome-friendly preparation may be a device or item. The device or item may be or comprise one of more of the household products described herein. The household product may be formulated as a solid, liquid, gas, or gel product. In some embodiments, the household product may comprise or be contained in a disposable sponge or wipes.

The household product which may be rendered biome-friendly by application of a biome-friendly preparation may be or include sport equipment, outdoor recreational equipment, exercise, or fitness equipment. For example, the household product may be or comprise a yoga mat, gym mat or flooring, gymnastics or wrestling mat, towel, sleeping bag, tent, sports bag or other carrying equipment, climbing equipment, cycling equipment, water sports equipment, snow sports equipment, weights, weight-training machines, cardiovascular exercise machines, and other sports-related equipment. The household product may be or include a sport, recreational, exercise, or fitness equipment cleaner, polisher, or conditioner.

The consumer product which may be rendered biome-friendly by application of a biome-friendly preparation may be, or include, or be disposed in a laundry product. For example, the consumer product may be or comprise a laundry detergent, bleach, dryer sheet, scent, stain remover, post-laundry finishing spray, or fabric softener. The consumer product may be, or include, or be disposed in a textile or fabric. The consumer product may comprise a textile or fabric having or containing the consumer product, for example, a textile or fabric to which the consumer product has been applied.

The consumer products of the disclosure may be, or include, or be disposed in a baby product. The baby product may be or include a car seat, stroller, carrier, nursery, diapering, nursing, bathroom, toy, training, or infant safety product or accessory. The baby product may be itself biome-friendly, may comprise one or more biome-friendly components, e.g., textiles or surfaces, or may be treated to be biome-friendly. In at least some embodiments, the baby product may be a bottle, baby bathing product, pacifier, teething product, crib, bassinet, swing, utensil, cup, plate, storage, clothing or diaper product.

The consumer products which may be rendered biome-friendly by application of a biome-friendly preparation may be, or include, or be disposed in a pet product. The pet product may be or include a pet health, safety, training, cleaning, grooming, bathing, pest management, crate carrier, bowl, collar, harness, leash, bed, accessory, litterbox, litter, diaper pad, toy, storage container, or treat product. The pet product may be a pet dietary supplement. The pet product may be itself biome-friendly, may comprise one or more biome-friendly components, e.g., textiles or surfaces, or may be treated to be biome-friendly.

The consumer products which may be rendered biome-friendly by application of a biome-friendly preparation may be, or include, or be disposed in a food or beverage product. The food or beverage product may be or include a dietary or nutritional supplement, i.e. a vitamin, mineral, specialty (e.g., fish oil, fiber, probiotic, prebiotic, glucosamine, chrondroitin, CoQ10), herbal, botanical, sports, or weight management supplement. The food or beverage product may be packaged in a biome-friendly material, for example, the packaging may render the consumer product biome-friendly. The food or beverage product may be a liquid, e.g., juice or smoothie, solid, e.g., a bar or powder, or gel food or beverage product. The food or beverage product may be formulated to be consumed with water or with other ingredients, for example, as a cooking supplement. The food or beverage product may be a cooking oil.

The consumer products which may be rendered biome-friendly by application of a biome-friendly preparation may be, or include, or be disposed in a personal product. The personal product may be or comprise an electronic device and/or a component thereof. The personal product may be or include, for example, a television, video game system, audio system, headphone, mobile phone, wearable device, tablet, e-reader, or technology accessory, i.e., a mobile technology accessory. The electronic device may be or include a computer, e.g., a desktop computer, laptop computer, or mobile computer. The electronic device may comprise one or more biome-friendly components, e.g., screen, keys, mouse or trackpad, remote control, removable case, fixed housing, or a textile, metal, or plastic component.

The personal product which may be rendered biome-friendly by application of a biome-friendly preparation may be or comprise an accessory. The personal product may be an accessory, e.g., a fashion accessory. The accessory may be or include, for example, a handbag, wallet, keychain, hat, scarf, belt, glove, mitten, jewelry, hair accessory, glasses, or sunglasses. The accessory may be or include, for example a backpack or luggage item. The accessory may be or comprise one or more biome-friendly components. In at least some embodiments, the accessory is a personal accessory.

The personal product which may be rendered biome-friendly by application of a biome-friendly preparation may be or comprise an office or school supply. The office or school supply may be or include, for example, office or school furniture, an electronic device, e.g., calculator, copy machine, printer, or telephone, notebook, notepad, book, pen, pencil, marker, or other writing utensil, eraser, scissors, ruler, tape, glue, sharpener, binder, paper clip, bookmark, flag, whiteboard, blackboard, or projector screen. The office or school supply may be or comprise one or more biome-friendly components.

Consumer products which may be rendered biome-friendly by the methods disclosed herein include articles of clothing. Articles of clothing such as, for example, shoes, shoe inserts, pajamas, sneakers, belts, hats, shirts, underwear, athletic garments, helmets, towels, gloves, socks, bandages, and the like, may also be treated with a biome-friendly preparation. Bedding, including sheets, pillows, pillow cases, and blankets may also be treated with a biome-friendly preparation.

In some aspects, the present disclosure provides a wearable article which has been treated with a biome-friendly preparation as disclosed herein. A wearable article may be a light article that can be closely associated with a user's body, in a way that does not impede ambulation. Examples of wearable articles include a wristwatch, wristband, headband, hair elastic, hair nets, shower caps, hats, hairpieces, adhesive plastic films and patches, adhesive microneedle patches and arrays, and jewelry. The wearable article treated with the biome-friendly preparation described herein may provide, e.g., at a concentration that provides one or more of a treatment or prevention of a skin disorder, a treatment or prevention of a disease or condition, a treatment or prevention of body odor, a treatment to supply a beneficial biproduct to a subject, or a treatment to inhibit microbial growth.

In some embodiments, the consumer product which has been rendered biome-friendly is a product intended to contact the hair, for example, a brush, comb, shampoo, conditioner, headband, hair elastic, hair nets, shower caps, hats, and hairpieces. Beneficial microorganisms released through the scalp, e.g., disposed on the hair, away from the skin surface, may be captured in a hat, scarf or face mask and directed into inhaled air.

Articles contacting the surface of a human subject, such as a diaper, may be rendered biome-friendly.

The method may comprise re-applying the biome-friendly preparation to the consumer product after a pre-determined period of time. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour. The pre-determined period of time may be less than 1 hour, for example, the pre-determined period of time may be 50 minutes, 45 minutes, 40 minutes, 35 minutes, 30 minutes, 25 minutes, 20 minutes, 15 minutes, 10 minutes, 5 minutes, 3 minutes, 2 minutes, or 1 minute. In certain instances, the pre-determined period of time may be less than 1 minute. The biome-friendly preparation may be applied after use of the consumer product gives rise to a benefit in re-application of the biome-friendly preparation.

The methods of rendering a consumer product biome-friendly may further comprise applying to the consumer product a preparation comprising microorganisms, as disclosed herein. The method may further comprise applying the preparation comprising microorganisms to the consumer product prior to applying the biome-friendly preparation, wherein the preparation comprising microorganisms is applied between about one of the following ranges: about 1-5, 5-10, 10-20, 20-30, 30-40, 40-50, 50-60 minutes, 2-5, 5-10, 10-15, 15-20, 20-25 hours, 2-5, 5-10, 10-15, days, 3-4, 5-10 weeks prior to applying the biome-friendly preparation.

The method may further comprise applying the preparation comprising microorganisms to the subject subsequent to applying the biome-friendly preparation, wherein the preparation comprising microorganisms is applied between about one of the following ranges: about 1-5, 5-10, 10-20, 20-30, 30-40, 40-50, 50-60 minutes, 2-5, 5-10, 10-15, 15-20, 20-25 hours, 2-5, 5-10, 10-15, days, 3-4, 5-10 weeks subsequent to applying the biome-friendly preparation.

The method may comprise not applying a non biome-friendly product or preparation prior to or subsequent to applying the preparation comprising microorganisms.

The methods may comprise applying a second or subsequent dose of the microorganisms to the consumer product. The second or subsequent dose may be applied after a pre-determined period of time. In some embodiments, the pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour. The pre-determined period of time may be less than 1 hour, for example, the pre-determined period of time may be 50 minutes, 45 minutes, 40 minutes, 35 minutes, 30 minutes, 25 minutes, 20 minutes, 15 minutes, 10 minutes, 5 minutes, 3 minutes, 2 minutes, or 1 minute. In certain instances, the pre-determined period of time may be less than 1 minute. The second or subsequent dose may be applied once it has been determined that a second or subsequent dose is needed, for example, to maintain the community of microorganisms.

### 7. Use of the Consumer Product

The product, e.g., consumer product, may be used one time (1×) per day, twice (2×) per day, three times (3×) per day, 4× per day, 5× per day, 6× per day, 7× per day, 8× per day, or more. The product may be used 1× per week, 2× per week, 3× per week, 4× per week, 5× per week, 6× per week, or 7× per week.

The amount of consumer product in the end-use container may be sufficient for no more than a pre-determined amount of applications, for example, X applications, wherein X is between about 1 and about 60, for example, between about 1-3, 4-6, 7-9, 10-13, 14-17, 18-21, 22-25, 26-29, 30-33, 34-37, 38-41, 42-45, 46-49, 50-53, 54-57, 58-60. The amount of consumer product in the end-use container may be sufficient for no more than a pre-determined amount of applications, for example, X applications, wherein X is between about 1 and about 180, for example, between about 1-60, 61-120, 121-180. The amount of consumer product in the end-use container may be sufficient for no more than a pre-determined amount of applications, for example, X applications, wherein X is between about 1 and about 750, for example, between about 1-100, 101-200, 201-300, 301-400, 401-500, 501-600, 601-750.

The amount of consumer product in the finished consumer product may be selected such that the consumer product is sufficient for no more than a pre-determined amount of applications, for example, X applications, wherein X is between about 1 and 180. In embodiments, X may be between about 1 and about 180, for example, between about 1-60, 61-120, 121-180. The amount of consumer product in the end-use container may be sufficient for no more than a pre-determined amount of applications, for example, X applications, wherein X is between about 1 and about 750, for example, between about 1-100, 101-200, 201-300, 301-400, 401-500, 501-600, 601-750.

For example, for a surface conditioner, the number of applications may be about 15-40, for example, 1 per day for about 4 weeks. For a cleaner, the number of applications may be about several per day, for about 4 weeks, for example about 15-90 applications.

The amount of consumer product in the finished consumer product may be selected such that the finished consumer product may be used for no more than about a pre-determined number of uses, for example, between about 500-750 uses, 200-500 uses, 100-200 uses, 50-100 uses, 40-50 uses, 30-40 uses, 20-30 uses, 10-20 uses, 5-10 uses, or 1-5 uses.

The amount of consumer product in the finished consumer product may be selected such that the finished consumer may be used for no more than about a pre-determined number of days, for example, between about 500-750 days, 300-500 days, 100-300 days, 50-100 days, 40-50 days, 30-40 days, 20-30 days, 10-20 days, 5-10 days, or 1-5 days. The pre-determined number of applications, uses, or days may be about 28.

The amount of consumer product in the finished consumer product may be selected such that the finished consumer product is sufficient for not more than X days of Y/day use, wherein X is between about one day and about 42 days (6 weeks), and Y is between about zero uses per day and about ten uses per day. For example, X days may be about 1-6, 7-10, 10-13, 14-17, 18-21, 22-25, 26-29, 30-33, 34-37, 38-42 days; and Y may be about 0-1, 2-4, 5-7, 8-10 uses per day.

The consumer product may have an expiration date. This may be a date upon which, after such date, the consumer product should not be used, e.g. it should be disposed of. The expiration date may be a deterioration-based expiration date. This date may be a date after which a product, e.g. a consumer product, is expected to degrade or become contaminated in some way that would make it unsuitable for its intended purpose. In some instances, it may be a date after which a product, e.g., a consumer product, has degraded or become contaminated.

An indication that the product has degraded or become contaminated may be provided by way of a symbol or one or more written words on the end-use container. In embodiments, an indicator located on or in the consumer product or consumer product packaging, e.g, end-use container that provides for some sign that the product has degraded or become contaminated. For example, this may be accomplished by way of a color indicator.

The expiration date may be a biome-compatible-based expiration date. This date may be a date after which a product, e.g. a consumer product, is expected to become contaminated in some way that would make it unsuitable for its intended purpose. In some instances, it may be a date after which a product, e.g., a consumer product, has become contaminated. An indication that the product has become contaminated may be provided by way of a symbol or one or more written words on the end-use container. In embodiments, an indicator located on or in the consumer product or consumer product packaging, e.g., end-use container that provides for some sign that the product has been contaminated. For example, this may be accomplished by way of a color indicator. A contaminant may refer to something that may make the product unsuitable for its intended use and may include any item that is not in the finished consumer product at the time of sealing the product and/or subsequent to sterilization of the product.

In some embodiments, the expiration date may be a date after which a product is expected to become contaminated in some way that would make it unsuitable for its intended purpose. For example, the expiration date may be a date after which the product is not safe for consumption. The expiration date may be a date after which the product is not able to provide a health benefit or provides a limited health benefit. The expiration date may be a date after which the product may not support engraftment of a beneficial microorganism or may support limited engraftment of the microorganism.

The finished consumer product may have an indication of expiration, or lifetime. This indication of expiration may be a recommended lifetime of the product, and it may be a preselected period of time. The preselected period of time may be expressed as a unit of time, for example, expressed in days, weeks, or months.

The finished consumer product may comprise an indication of expiration, or lifetime, e.g., recommended lifetime, after the preselected period of time, e.g., expressed in days, that is less than X days from the date of one of the following: manufacturing, filling, sealing, sterilization, shipping, releasing into commerce, or selling. In embodiments, X may be about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days. In certain embodiments, X may be about 28 days, e.g., 28 days (4 weeks).

In embodiments, X may be related to the opening or unsealing of the finished consumer product, or the first use of the finished consumer product. For example, about 5-7 days, about 5-10 days, about 7-14 days, about 14-21 days, about 21-28 days, about 28-35 days, about 35-42 days, about 42-49 days, about 49-56 days, about 56-63 days, about 63-70 days, about 70-77 days, about 75-100 days, about 100-150 days, about 150-200 days, about 200-300 days, about 300-400 days, about 400-750 days. In certain embodiments, X may be about 28 days, e.g., 28 days.

In embodiments, the indication of expiration, or lifetime, e.g., recommended lifetime, is expressed as a preselected number of uses or applications. The indication may be expressed as a preselected number between about 5-7, about 5-10, about 7-14, about 14-21, about 21-28, about 28-35, about 35-42, about 42-49, about 49-56, about 56-63, about 63-70, about 70-77, about 75-100, about 100-150, about 150-200, about 200-300, about 300-400, about 400-750 days.

In embodiments, the finished consumer product may have an expiration date, or lifetime, e.g., recommended lifetime that is expressed:

a) in units of time, e.g., days, from a preselected event, e.g., unsealing of said finished consumer product or the first use of said finished consumer product; and/or

b) as the number of uses or applications.

The product may be used in conjunction, e.g., in combination, with a microorganism-containing product, e.g., a beneficial microorganism containing product, e.g., a non-pathogenic bacteria containing product.

Methods may be provided that allow for maintenance of microorganisms, e.g., beneficial microorganisms, e.g. non-pathogenic bacteria, using one or more consumer products, e.g, finished consumer products disclosed herein.

In certain instances, methods may comprise application of microorganisms, e.g., beneficial microorganisms, e.g. non-pathogenic bacteria, prior to or subsequent to use of one or more consumer products disclosed herein.

The methods may comprise waiting a period of time, e.g., a pre-determined period of time before using the consumer product. The period of time may comprise a period of time in which a non-biome-friendly consumer product is not used.

Use of the consumer product, e.g., finished consumer product may occur on a regular basis, e.g., every day, every week, every month, or in one of the following ranges, every 1-2 days, every 2-5 days, every 5-10 days, every 10-15 days, every 15 to 30 days.

In embodiments, no non-biome friendly consumer product may be used by the subject in the period between the last use of the consumer product and the next use of the consumer product and/or the microorganisms, e.g., beneficial microorganisms. In embodiments, no non-biome friendly consumer product may be applied to the item in the period between the last application of the consumer product and the next application of the consumer product and/or the microorganisms, e.g., beneficial microorganisms.

The product may be used in conjunction, e.g., in combination, with a bacteria-containing product, e.g., an ammonia oxidizing microorganism (AOM) containing product, e.g., an N. eutropha containing product, e.g., an N. eutropha D23 containing product.

Methods may be provided that allow for maintenance of microorganisms, e.g., AOM, e.g. N. eutropha, e.g., an N. eutropha D23, using one or more consumer products, e.g, finished consumer products disclosed herein.

Methods may comprise application of microorganisms, e.g., AOM, e.g. N. eutropha, e.g., an N. eutropha D23, prior to or subsequent to use of one or more consumer products disclosed herein.

### 8. Methods of Making a Biome-Friendly Consumer Product

Methods of making a consumer product are provided that may comprise disposing a consumer product in an end-use container to form a filled end-use container. The filled end-use container may then be treated to kill or inactivate bacteria, which may then provide the finished consumer product. The killing or inactivation of bacteria may be accomplished through a sterilization technique described herein.

Other methods may be provided, that allow for manufacturing or making of a biome-friendly consumer product. The method may comprise selecting a first component from a list of biome-friendly components; selecting a second component from a list of biome-friendly components; and integrating the first and second components (or more), thereby making a biome-friendly consumer product.

The method may further comprise selecting a third component from a list of biome-friendly components. The method may further comprise selecting a fourth component from a list of biome-friendly components. Additional components that may be selected include, e.g., fifth, sixth, and seventh components. Any of the first through seventh or subsequent components may be or comprise a cleanser, surfactant, viscosity modifier, humectant, fragrance, conditioner, lipid layer enhancer, or emulsifier.

Once the combination has been obtained, for example, through integration of the components, treatment of the combination, e.g., sterilization may be accomplished, before or after sealing the consumer product in an end-use container.

Methods may also be provided where a first and second component is provided, wherein each has been shown to be biome-friendly. The method may comprise integrating the first and second component to form a consumer product. Determination if the consumer product is biome-friendly may be accomplished through the methods disclosed herein, thereby making a biome-friendly consumer product.

Further methods may be provided of manufacturing, e.g., evaluating a consumer product, as disclosed herein. The method may comprise providing a product having at least 2 components selected from Table 3. The method may comprise acquiring an evaluation of whether the consumer product is safe for microorganisms, e.g., bacteria, e.g., beneficial bacteria, to be used in the vicinity of the user, thereby manufacturing the consumer product. The evaluation may comprise evaluating the viability of a beneficial microorganism, as disclosed herein, or of the ability of the microorganism to produce a beneficial byproduct after contact with the consumer product, or one or more components of the consumer product.

Sterilization may be used to prepare the consumer product for use, e.g., for sale, or for consumer use. The product may be sterilized at one or more steps in the manufacturing process.

Sterilization may be performed by irradiation or by heat. A consumer product may be provided that comprises a sterilized, e.g., irradiated, e.g., heat sterilized, product disposed in a container. The container may be preferably a microorganism proof container, or a container that may be sealed from the outside environment.

Sterilization, e.g., heat sterilization or irradiation, may be performed on single components of the product, a combination of two or more components of the product, or all of the components of the product, to provide the consumer product. Sterilization may be performed at any step during the manufacturing process. For example, sterilization may be performed prior to disposing in a container, e.g., prior to being disposed in a container for commercial release. Sterilization may be performed after disposing the consumer product, or one or more components of the consumer product in a container, e.g., in a container for commercial release. Sterilization may be performed after disposing the consumer product in a container, e.g., in a container for commercial release prior to or after sealing the container for commercial release.

Determination that a consumer product is sterile may be measured by as described below. The consumer product may be determined to be sterile, when at least 10, 20, 30, 40, 50, 60, 70, 80, 90, 95, 99, or 99.9% of the microorganisms, e.g., bacteria, mold, fungus, or viruses are dead, incapable of cell division, or have radiation induced DNA damage sufficient to inhibit cell division. The consumer product may be determined to be sterile, when all microorganisms, e.g., bacteria, mold, fungus, or viruses are dead, incapable of cell division or have radiation induced DNA damage sufficient to inhibit cell division. The consumer product, e.g., packaged consumer product, may be determined to be sterile, when the Sterility Assurance Level (SAL) of less than about 10⁻¹, 10⁻², 10⁻³, 10⁻⁴, 10⁻⁵, 10⁻⁶, 10⁻⁷, 10⁻⁸, 10⁻⁹ is achieved. The consumer product may be determined to be sterile, when induced DNA damage is sufficient to inhibit cell division.

In certain embodiments, sterile may be considered an absolute state in which everything, e.g., all microorganisms, bacteria, mold, fungus, and viruses, are dead, e.g., dead in view of the limits to a given testing methodology, e.g., Sterility Assurance Level. The sterility testing may be performed as outlined in the U.S. Pharmacopeia at USP31-NF26, page 670<1211> (Pharmacopeial Forum: Volume No. 30(5), page 1729) “Sterilization and Sterility Assurance of Compendial Articles” and USP31-NF26, page 85<71>“Sterility Tests”, each of which is incorporated herein by reference in their entireties. The consumer product may be considered sterile, when the end-use container is unopened, e.g., the factory seal has not been broken.

Irradiation, e.g., gamma irradiation, may be performed on single components of the product, a combination of two or more components of the product, or all of the components of the product, providing the consumer product. Irradiation may be performed at any step during the manufacturing process. For example, irradiation may be performed prior to disposing in a container, e.g., prior to being disposed in a container for commercial release. Irradiation may be performed after disposing the consumer product, or one or more components of the consumer product in a container, e.g., in a container for commercial release. Irradiation may be performed after disposing the consumer product in a container, e.g., in a container for commercial release prior to or after sealing the container for commercial release.

The consumer product may be irradiated, e.g., with ionizing radiation, e.g., with gamma rays, e.g., with x-rays, e.g., from an isotope, e.g., cobalt 60, or with ultraviolet, e.g., ultraviolet C (UVC). The consumer product may be irradiated in order to sufficiently provide a sterile product. In embodiments, as defined in Chapter 1211 of Sterilization and Sterility Assurance Compendial Articles of the U.S. Pharmacopeia (referenced in the preceding paragraph and incorporated herein by reference in its entirety), “within the strictest definition of sterility, a specimen would be deemed sterile only when there is complete absence of viable microorganisms from it.” The sterile product may be characterized in that it is free, e.g., substantially free, from microorganisms, e.g., bacteria, e.g., fungi, capable of growth, e.g., as determined by U.S.P. 71 Sterility Testing Methods and Standards (referenced in the preceding paragraph and incorporated herein by reference in its entirety). For example, when challenged for microorganisms capable of growth, said consumer product shows no growth on appropriate culture media, e.g., when said microorganisms are measured by U.S.P. 71 Sterility Testing Methods and Standards.

The consumer product may comprise an exogenously added additive selected from an oxidant, e.g., a naturally occurring oxidant, a free radical scavenger, or a free radical quencher. Free radical scavengers or oxidants may be added to the consumer product as part of the sterilization procedure. The free radical scavengers or oxidants may include one or more of the following tocopherols, tocotrienols, ascorbic acid, polyphenols, isoflavones, coenzyme Q10, and other similar compounds. The free radical scavengers or oxidants may be selected from the group consisting of lipid soluble or water soluble free radical scavengers, or combinations thereof.

The consumer product may contain a plurality of components and may be irradiated after integration of the plurality of components. In embodiments, the consumer product is irradiated after said consumer product is disposed in the end-use container. In embodiments, the consumer product may be irradiated after closure of the end-use container. In embodiments, the consumer product may be irradiated after sealing of the end-use container. In embodiments, the consumer product is irradiated prior to closure of the end-use container. In embodiments, the consumer product, or the end-use container comprises an indicator that may indicate if said consumer product has been irradiated.

In embodiments, the consumer product may arrive to a processing facility. The product may be received by lot and product code, which allows for run generation, scheduling, processing, certification, and release of product for shipment. The product may be loaded into a carrier according to suitable conditions and configurations. Dosimeters may be placed in, around, or outside of the carrier. The product may then be exposed to irradiation, for example cobalt 60, for example, cobalt 60 source rack. Dosimeters may be analyzed after irradiation of the product is complete, to confirm that the required dose has been delivered. The dosimeters may be provided to validate the process. If the processing history is acceptable, and/or according to specifications, the product may be released and shipped for use or further distribution.

In embodiments, the irradiation may be performed as a batch process. In other embodiments, the irradiation may be performed as a continuous process. A single unit, or multiple units, e.g., packaged boxes of units may be irradiated in either a batch process or a continuous process.

In embodiments, the radiation absorbed dose is provided as kGy. For example, the radiation absorbed in order to provide sterilization may be between about 10 kGy to about 25 kGy. In embodiments, the radiation absorbed may be between about 15 kGy to about 25 kGy.

In embodiments the Sterility Assurance Level (SAL) of the consumer product may be less than about 10⁻¹, 10⁻², 10⁻³, 10⁻⁴, 10⁻⁵, 10⁻⁶, 10⁻⁷, 10⁻⁸, 10⁻⁹, achieved through the conditions described herein. In embodiments, a SAL of 10⁻³ or greater may be achieved.

The time of exposure is provided depending on the density of the product being irradiated. For example, the time of exposure for a single unit of product, may be less than the time of exposure of multiple units of product, due to differences in density.

In embodiments, heat sterilization may be utilized. The consumer product may be heated, e.g., by microwave oven or autoclave. The heating may be sufficient to provide a sterile product. The sterile product may be characterized in that it is substantially free from microorganisms, e.g., bacteria, e.g., fungi capable of growth, consistent with U.S.P. Chapter 1211, as determined by U.S.P. 71 Sterility Testing Methods and Standards, each of which is referenced above and incorporated herein in their entireties. The consumer product may be determined to be sterile, when challenged for microorganisms capable of growth, said consumer product shows no growth e.g., when said microorganisms are measured by a testing methodology described herein.

In embodiments, the consumer product may contain a plurality of components and is heated after integration of the plurality of components. In embodiments the consumer product is heated after the consumer product is disposed in the end-use container. In embodiments, the consumer product is heated after closure of the end-use container. In embodiments, the consumer product is heated after sealing the end-use container. In embodiments, the consumer product is heated prior to closure of the end-use container.

In embodiments, the consumer product or the end-use container comprises an indicator that indicates if said consumer product has been heated. In embodiments, the consumer product is heated at or above 121 degrees Celsius for at least 15 minutes during or after formulation, e.g., after combining, integrating, mixing, or filling, e.g., after disposing, or after sealing the end-use container. The consumer product may not be heated above 140 degrees Celsius during or after formulation, e g., combining, integrating, mixing, or filling, e.g., after disposing, or after sealing the end-use container. This may be due to the end-use container and/or the contents of the end-use container, e.g., the consumer product undergoing degradation above a temperature of 140 degrees Celsius. The temperature at which the consumer product and/or container may experience degradation may be determined with respect to the material of the consumer product and/or container and may be greater or less than 140 degrees Celsius.

The consumer product or end-use container may include an indicator that indicates if the consumer product was heated, sterilized, or irradiated. The indicator may be positioned on an exterior surface of the consumer product or the packaging, e.g., container, of the consumer product. The indicator may be disposed within the consumer product or the packaging, e.g., container of the consumer product. The indicator may include a color indicator. In some embodiments, the indicator comprises a dosimeter or temperature sensor. The indicator may comprise a mechanism capable of measuring microorganism presence.

The manufacturing or making method of the consumer product may further include employing other techniques to preserve the consumer product, for example, without including an anti-bacterial preservative. In some embodiments, the consumer product may have low quantities of bio-available water to preserve the consumer product. The consumer product may be concentrated to limit bio-available water. The consumer product may be heated or dried to reduce water concentration. In some embodiments, the consumer product may be treated such that a user is instructed to hydrate or dilute the consumer product. The consumer product may be diluted between 4× and 10× before use. In some embodiments, the consumer product is substantially free of bio-available water. The consumer product may be diluted between 10× and 100× before use.

### 9. Evaluating a Consumer Product

A test may be performed in order to confirm that the product, e.g., consumer product, or a component thereof is free of microorganisms, e.g., bacteria or fungus.

A method of evaluating a product, e.g., a consumer product, or a component thereof, may comprise contacting a portion, e.g., in certain embodiments a portion or aliquot, of the product with a test organism and evaluating the effect of the consumer product on the test organism. The test organism may be any microorganism which can be tested for viability. A microorganism may be tested for viability, for example, by culture or by determining an amount of byproduct production. In some embodiments, the test organism may be an ammonia oxidizing microorganism. Evaluating may comprise evaluating the effect of the consumer product on the ability of the test organism, e.g., ammonia oxidizing microorganism, to produce a byproduct, e.g., nitrite.

Evaluating the product may comprise determining if the ability of the test organism, e.g., ammonia oxidizing microorganism, to produce a byproduct, e.g., nitrite, meets a preselected criteria, e.g., at least has the ability to recover so as to produce the byproduct over a given period of time as shown in the Examples disclosed herein.

Methods disclosed herein may comprise exposing the consumer product to challenge with a microbe, e.g., a bacteria or fungus. In certain instances, the consumer product may be applied to a surface and the surface may be exposed to challenge with a microbe, e.g., a bacterium or fungus. Challenging the product may comprise exposing a surface of the consumer product to challenge with a microbe, e.g., a bacterium or fungus. The method may further comprise determining whether the consumer product will support growth of the microbe. For example, the method may comprise determining whether the product will support growth of the microbe by U.S.P. 51, Antimicrobial Effectiveness Testing or U.S.P. 62, Microbial Examination of Nonsterile Products. An ingredient or consumer product which has been determined to survive the challenge or support growth of the microbe may be identified as biome-friendly. In some embodiments, the ingredient or consumer product may be certified as being biome-friendly. The ingredient or consumer product may further be identified as being certified biome-friendly.

Evaluating the product may provide for the finished consumer product to be identified as “tested and confirmed as biome-friendly” or “tested and confirmed as biome-compatible.” Evaluating the product may provide for the finished consumer product to be identified as “tested and confirmed to have the live test organisms.” Evaluating may provide for the finished consumer product to be identified as “tested and confirmed to have live ammonia oxidizing bacteria.”

Methods may be provided to select an ammonia oxidizing bacteria-friendly ingredient. In embodiments, a method of selecting a biome-friendly ingredient, e.g., ammonia oxidizing microorganism-friendly ingredient is provided comprising obtaining a sample of a test organism, e.g., ammonia oxidizing microorganisms (AOM), and contacting the AOM with a product or component, e.g., an excipient, for a first pre-determined time period to provide an incubated culture. The method may further comprise collecting an aliquot of the incubated culture and measuring a concentration of byproduct, e.g., nitrite, in a supernatant of the incubated culture. The method may further comprise identifying the product or component, e.g., excipient, as a microbiome-friendly, e.g., ammonia oxidizing bacteria-friendly, ingredient based on the concentration of byproduct, e.g., nitrite in the supernatant of the incubated culture.

In certain embodiments, the consumer product is not a liquid product, for example, the consumer product may be or comprise a solid item. In such embodiments, the method may comprise contacting the test organism with the item for a pre-determined time period and collecting a sample of microorganisms on the surface of the item to incubate and measure a concentration of byproduct.

The method may further comprise contacting the test organism, e.g., AOM from the incubated culture with NH₄⁺, and measuring the recovered AOM cell sample for at least one of an OD₆₀₀ value and nitrite accumulation after a second pre-determined time period. The method may further comprise identifying the product or component, e.g., excipient, as an ammonia oxidizing microorganism-friendly ingredient based on at least one of the OD₆₀₀ value and nitrite accumulation in the recovered AOM cell sample.

The pre-determined final concentration of product or component, e.g., excipient, may be between about 0% and about 100%. The first pre-determined time period may be at least one of about 1 minute, about 10 minutes, about 60 minutes, about 2 hours, about 4 hours, about 6 hours, about 12 hours, and about 24 hours. The second pre-determined time period may be at least one of about 1 minute, about 10 minutes, about 60 minutes, about 2 hours, about 4 hours, about 6 hours, about 12 hours, about 24 hours, about 36 hours, about 48 hours, about 72 hours, about 96 hours.

Depending on the ingredient, excipient, or composition tested, the concentration of byproduct, e.g., nitrite, measured may allow for identification of a biome-friendly ingredient, excipient, or composition, e.g., an ammonia oxidizing microorganism-friendly ingredient, excipient, or composition. In certain embodiments, byproduct, e.g., nitrite, production measured may be at or above a certain value of byproduct, e.g., nitrite, production in order to qualify the ingredient or excipient as a biome-friendly ingredient, excipient, or composition. In certain embodiments, this nitrite production concentration may be measured at a certain period of time after contacting the AOM with the incubated culture with NH₄⁺. The period of time may be at least 1 minute, 5, 10, 20, 30, 40, 50, 60, 120, minutes. The period of time may be at least 3, 4, 5, 10, 15, 20, 24, 36, 48, 72, or 96 hours, or more.

The byproduct, e.g., nitrite, production may be at or above a certain value, e.g., at or above 10 micromolar, 20, 50, 100, 200, 300, 400, 500, 600, 700, 800, 900, 1000, 1500, 2000, 3000 micromolar concentration, or more, in order for the ingredient, excipient, or composition to be identified as a biome-friendly ingredient.

In embodiments, a byproduct, e.g., nitrite, production of greater than 1000 micromolar, measured at the end of a 48 hour period would be indicative of a biome-friendly ingredient, excipient, or composition. In other embodiments, byproduct, e.g., nitrite, production of greater than 100 micromolar, measured at the end of a 48 hour period would be indicative of a biome-friendly ingredient, excipient, or composition. In other embodiments, byproduct, e.g., nitrite, production of greater than 10 micromolar, measured at the end of a 48 hour period would be indicative of a biome-friendly ingredient, excipient, or composition.

Methods of producing a biome-friendly composition may comprise acquiring knowledge that a compound, or more than one compound is friendly to microorganisms, e.g., beneficial microorganisms, e.g., ammonia oxidizing microorganisms, and packaging the compound, or more than one compound in an end-use container, to provide a product, e.g., a consumer product that is biome-friendly.

Methods of producing a biome-friendly composition may comprise acquiring knowledge that a compound is friendly to ammonia oxidizing bacteria, and combining the compound with ammonia oxidizing bacteria to provide a consumer product.

In accordance with another aspect, there is provided a kit for evaluating a consumer product. The kit may comprise a biome-friendly consumer product and a preparation comprising the test organism. The kit may further comprise instructions to evaluate the effect of the consumer product on the test organism, as previously described.

In some embodiments, the test organism may comprise ammonia oxidizing microorganisms (AOM). In such embodiments, the kit may further comprise a reagent to detect nitrite production.

10. Use of the Product in Conjunction with Beneficial Microorganisms

The products disclosed herein may be used in conjunction with application of a beneficial bacteria, e.g., a non-pathogenic bacteria.

In accordance with one or more embodiments, essentially any beneficial microorganism can be used or implemented. The beneficial microorganism, e.g., non-pathogenic bacteria may generally be autotrophic or heterotrophic. These microorganisms have beneficial properties, e.g., in connection with various cosmetic and therapeutic uses, in accordance with one or more embodiments described herein.

Beneficial microorganisms, e.g., non-pathogenic bacteria may be selected for meeting a predetermined criteria or reference. For example, non-pathogenic bacteria may be selected for ability to compete with pathogenic bacteria in the microbiome of a subject. In some embodiments, non-pathogenic bacteria may be selected for their ability to produce byproducts that inhibit growth or reproduction of pathogenic bacteria in the microbiome of a subject.

Certain bacteria, for example, including those commonly present in the microbiome of a subject, may inhibit the growth and reproduction of pathogenic bacteria therein. For example, the most significant pathogenic bacteria of the lung include M. catarrhalis, H. influenzae, and S. pneumoniae. While not wishing to be bound by any particular theory, certain non-pathogenic bacteria from the respiratory microbiome may produce anti-inflammatory and antimicrobial particles, including, e.g., interleukin 10 (IL-10), FOXP3, and secretory immunoglobulin A (sIgA), and induce a Th1 response, which inhibits pathological growth.

The non-pathogenic bacteria of this disclosure may be from a genus selected from the group consisting of Prevotella, Sphingomonas, Pseudomonas, Acinetobacter, Fusobacterium, Megasphaera, Veillonella, Staphylococcus, or Streptococcus, and combinations thereof. Non-pathogenic bacteria of such genera, for example, may inhibit the growth of pathogenic bacteria by colonizing the subject or delivering one or more product or byproduct to the subject.

In some embodiments, the non-pathogenic bacteria may inhibit growth and/or reproduction of pathogenic bacteria, e.g., S. pneumoniae and S. aureus. While commonly benign when present in the nasal passage microbiome, these bacteria can sometimes become pathogenic and cause severe disease. It is not well understood what causes a pathogenic state of these bacteria, however, it is generally believed that colonization is a requirement for infection. While not wishing to be bound by any particular theory, certain non-pathogenic bacteria, for example, C. accolens, may inhibit pneumococcal growth in the microbiome by releasing fatty acids that inhibit the pathogenic bacteria. It is believed that C. accolens releases lipase LipS1 which hydrolyzes triacylglycerols, e.g., triolein, commonly found lining the nasal passages to release oleic acid, a fatty acid which inhibits pneumococcal growth.

The non-pathogenic bacteria of this disclosure may be from a genus selected from the group consisting of Staphylococcus, Corynebacterium, Propionibacterium, Rhodococcus, Microbacterium, or Streptococcus, and combinations thereof. Non-pathogenic bacteria of such genera, for example, may inhibit the growth of pathogenic bacteria by colonizing the nasal passages or delivering one or more product or byproduct to the nasal passages.

In some embodiments, the non-pathogenic bacteria may comprise one or more bacterium of the genus selected from the group consisting of Bacillus, Lactobacillus, Lactococcus, Streptomyces, Faecalibacterium, Bacteroides, or Bifidobacter and combinations thereof, e.g., L. rhamnosus, F. prausnitzii, and B. fragilis.

The non-pathogenic bacteria may be provided as a community of bacteria. For instance, the non-pathogenic bacteria may comprise a community of species of bacteria. In some embodiments, the community of species may be one that is generally found in a mammalian microbiome, e.g., a human microbiome. A sample of a human microbiome may reveal an exemplary community of species of bacteria, which overlaps with the community of species of bacteria provided herein. In some embodiments, the community of species may be one that is generally found in a healthy mammalian microbiome. The community of species may be one that is present or can be found in a mammalian gastrointestinal, respiratory, or skin microbiome.

In some embodiments of the disclosure, the beneficial microorganism may be provided in the form of a preparation. The beneficial microorganism may be used in conjunction or in combination with the consumer products of the present disclosure. For example, beneficial microorganisms or a preparation of beneficial microorganisms may be in the form of a product that is separate from the consumer product, or may be provided together with the consumer product, e.g., in the same or different end-use container.

The microorganisms may be associated with a variety of consumer and therapeutic products, and examples of such products are set out below. In some embodiments, the microorganism associated with a product is admixed with the product, for example, spread evenly throughout the product, and in some embodiments, the microorganism associated with a product is layered on the product.

In some embodiments, the microorganism is associated with a powder. Powders are typically small particulate solids that are not attached to each other and that can flow freely when tilted. Exemplary powders for consumer use include talcum powder and some cosmetics (e.g., powder foundation, including pressed powders). Other powders may be contemplated for use in conjunction with microorganism systems and methods of the present disclosure.

In some embodiments, the microorganism is associated with a cosmetic. The cosmetic may be a substance for topical application intended to alter a person's appearance, e.g., a liquid foundation, a powder foundation, blush, or lipstick. The cosmetic may be any substance recited in the Food and Drug Administration regulations, e.g., under 21 C.F.R. § 720.4.

The products disclosed herein may be used in conjunction with application of an ammonia oxidizing microorganism.

Autotrophic ammonia oxidizing microorganisms, which may be referred to herein as AOMs or AOM, are obligate autotrophic microorganisms, e.g., bacteria, as noted by Alan B. Hooper and A. Krummel at al. Alan B. Hooper, Biochemical Basis of Obligate Autotrophy in Nitrosomonas europaea, Journal of Bacteriology, February 1969, p. 776-779. Antje Krummel et al., Effect of Organic Matter on Growth and Cell Yield of Ammonia-Oxidizing Bacteria, Arch Microbiol (1982) 133: 50-54. These microorganisms derive all metabolic energy only from the oxidation of ammonia to nitrite with nitric oxide (NO) as an intermediate product in their respiration chain and derive virtually all carbon by fixing carbon dioxide. They are incapable of utilizing carbon sources other than a few simple molecules.

Ammonia oxidizing microorganisms (AOM) are widely found in the environment, and in the presence of ammonia, oxygen and trace metals will fix carbon dioxide and proliferate. AOM may be slow growing and toxic levels of ammonia may kill fish and other organisms before AOM can proliferate and reduce ammonia to non-toxic levels. Slow growth of AOM also may delay the health benefits of the NO and nitrite the AOM produce when applied to the skin.

Supplementing the aquarium, skin, or process with sufficient viable AOM grown and stored for that purpose is desired. AOM do not form spores, so storage in the dry state with high viability is difficult, and storage in the wet state leaves them metabolically active.

This disclosure provides a composition having ammonia oxidizing microorganisms, or more specifically having one genus of ammonia oxidizing microorganism, or more specifically, having one species of ammonia microorganism, e.g., N. eutropha, and one other type of organism, and no other types of organism. In other examples, the composition has ammonia oxidizing microorganisms, or more specifically has one genus of ammonia oxidizing microorganism, or more specifically, having one species of ammonia microorganism, e.g., N. eutropha and 2, 3, 4, 5, 6, 7, 8, 9, or 10 other types of organism, and no other types of organism. Suitable ammonia-oxidizing microorganisms for this purpose include ammonia oxidizing bacteria, e.g., those in the genera Nitrosomonas, Nitrosococcus, Nitrosospira, Nitrosocystis, Nitrosolobus, or Nitrosovibrio.

Decay of nitrifying capacity during storage of ammonia oxidizing bacteria for wastewater treatment has been studied, as for example (Munz G, Lubello C, Oleszkiewicz J A. Modeling the decay of ammonium oxidizing bacteria. Water Res. 2011 January; 45(2): 557-64. Oi: 10.1016/j.watres.2010.09.022.)

Growth, prolonged storage, and restoration of activity of Nitrosomonas is discussed by Cassidy et al. (U.S. Pat. No. 5,314,542) where they disclose growing Nitrosomonas, removing toxic waste products, storing in sterile water of appropriate salinity for periods of time up to one year, and then reviving by adding buffer (CaCO₃) and 200 ppm, of ammonium, which reviving takes 72 hours.

As disclosed herein, if AOM are kept under conditions of low carbon dioxide but with sufficient oxygen and ammonia, where they accumulate polyphosphate for a period of about one doubling time (˜10 hours), then they accumulate sufficient polyphosphate to greatly extends their storage viability, storage time and accelerate their revival both with and without addition of buffer and ammonia.

The presence of sufficient stored polyphosphate allows AOM the ATP resources to maintain metabolic activity even in the absence of ammonia and oxygen, and to survive insults that would otherwise be fatal.

As obligate autotrophs, AOM synthesize protein via the fixing of CO₂ using the energy and reducing equivalents generated by the oxidation of ammonia to nitrite. Growth requires ammonia, oxygen, minerals and carbon dioxide.

Nitrosomonas may exist in several metabolic states, according to “Polyphosphate and Orthophosphate Content of Nitrosomonas europaea as a Function of Growth” by K. R. Terry and A. B. Hooper, Journal of Bacteriology, July 1970, p. 199-206, Vol. 103, No. I.

The AOMs contemplated in this disclosure may comprise mutations relative to wild-type AOMs. These mutations may, e.g., occur spontaneously, be introduced by random mutagenesis, or be introduced by targeted mutagenesis. For instance, the AOMs may lack one or more genes or regulatory DNA sequences that wild-type AOMs typically comprise. The AOMs may also comprise point mutations, substitutions, insertions, deletions, and/or rearrangements relative to the sequenced strain or a wild-type strain. The AOMs may be a purified preparation of optimized AOMs.

In certain embodiments, the AOMs are transgenic. For instance, it may comprise one or more genes or regulatory DNA sequences that a wild-type ammonia oxidizing microorganism lacks. More particularly, the ammonia oxidizing microorganism may comprise, for instance, a reporter gene, a selective marker, a gene encoding an enzyme, or a promoter (including an inducible or repressible promoter). In some embodiments the additional gene or regulatory DNA sequence is integrated into the microorganism's chromosome; in some embodiments the additional gene or regulatory DNA sequence is situated on a plasmid.

In some preferred embodiments, the AOMs differ by at least one nucleotide from naturally occurring microorganisms. For instance, the AOMs may differ from naturally occurring microorganisms in a gene or protein that is part of a relevant pathway, e.g., an ammonia metabolism pathway, a urea metabolism pathway, or a pathway for producing nitric oxide or nitric oxide precursors. More particularly, the AOMs may comprise a mutation that elevates activity of the pathway, e.g., by increasing levels or activity of an element of that pathway.

The above-mentioned mutations can be introduced using any suitable technique. Numerous methods are known for introducing mutations into a given position. For instance, one could use site-directed mutagenesis, oligonucleotide-directed mutagenesis, or site-specific mutagenesis. Non-limiting examples of specific mutagenesis protocols are described in, e.g., Mutagenesis, pp. 13.1-13.105 (Sambrook and Russell, eds., Molecular Cloning A Laboratory Manual, Vol. 3, 3.sup.rd ed. 2001). In addition, non-limiting examples of well-characterized mutagenesis protocols available from commercial vendors include, without limitation, Altered Sites® II in vitro Mutagenesis Systems (Promega Corp., Madison, Wis.); Erase-a-Base®System (Promega, Madison, Wis.); GeneTailor™ Site-Directed Mutagenesis System (Invitrogen, Inc., Carlsbad, Calif.); QuikChange® II Site-Directed Mutagenesis Kits (Stratagene, La Jolla, Calif.); and Transformer™ Site-Directed Mutagenesis Kit (BD-Clontech, Mountain View, Calif.).

In some embodiments of the disclosure, the ammonia oxidizing microorganism may be provided in the form of a preparation. The ammonia oxidizing microorganism may be used in conjunction or in combination with the consumer products of the present disclosure. For example, AOMs or a preparation of AOMs may be in the form of a product that is separate from the consumer product, or may be provided together with the consumer product, e.g., in the same or different end-use container. Ammonia oxidizing microorganisms may be associated with a variety of consumer and therapeutic products, and examples of such products are set out below. In some embodiments, the ammonia oxidizing microorganism associated with a product is admixed with the product, for example, spread evenly throughout the product, and in some embodiments, the ammonia oxidizing microorganism associated with a product is layered on the product.

In some embodiments, the ammonia oxidizing microorganism is associated with a powder. Powders are typically small particulate solids that are not attached to each other and that can flow freely when tilted. Exemplary powders for consumer use include talcum powder and some cosmetics (e.g., powder foundation, including pressed powders). Other powders may be contemplated for use in conjunction with ammonia oxidizing microorganism systems and methods of the present disclosure.

In some embodiments, the ammonia oxidizing microorganism is associated with a cosmetic. The cosmetic may be a substance for topical application intended to alter a person's appearance, e.g., a liquid foundation, a powder foundation, blush, or lipstick. The cosmetic may be any substance recited in the Food and Drug Administration regulations, e.g., under 21 C.F.R. § 720.4.

The preparation can, for example, be administered in form suitable to provide local therapeutic treatment or systemic therapeutic treatment. Preparations disclosed herein may be administered to treat a local inflammatory disease, a symptom of a local or systemic inflammatory disease, or a side effect caused by a local or systemic inflammatory disease. Examples of systemic conditions that may be treated with preparations disclosed herein include headaches, cardiovascular diseases, connective tissue disorders, inflammation, immune responses and autoimmune disorders, liver diseases, infections, neurological diseases, psychiatric disorders, nitric oxide disorders, urea cycle disorders, congestion, vasodilation disorders, skin diseases, ophthalmic disorders, wound healing, bowel disorders, reactions to insect bites, and certain viral, bacterial, and fungal infections.

For instance, systemic conditions that may be treated with preparations disclosed herein include cardiovascular diseases such as cardioprotection, heart failure, hypertension, pulmonary arterial hypertension; immune responses and autoimmune disorders such as alopecia and vitiligo; liver diseases such as non-alcoholic fatty liver disease (NAFLD), non-alcoholic steatohepatitis (NASH); neurological diseases and psychological disorders such as depression, insomnia, and diabetic neuropathy; nitric oxide disorders such as erectile dysfunction; wound healing, e.g., from bed sores and nursing home care, burns, diabetic ulcers e.g., foot ulcer, venous leg ulcer, biofilm, and mouth sores; skin diseases and disorders such as hyperhydrosis, pruritus, helomas, and subtypes of helomas; ophthalmic disorders such as blepharitis, dry eye, macular degeneration, and glaucoma; bowel disorders such as gluten sensitivity, irritable/inflammatory bowel disease, Crohn's disease, colitis, and necrotizing enterocolitis; auditory diseases such as tinnitus, reduced hearing, vertigo, pruritus, swimmer's ear, and congenital abnormalities; and vasodilation disorders such as Renaud's disease, thermoregulation, and migraines. Various connective tissue disorders may also be treated. Certain viral, bacterial, and fungal infections may be treated with the preparations disclosed herein, including infections caused by human papillomavirus (HPV), yeast infections, tinea versicolor, tinea unguium, tinea pedis/fungus, tinea cruris, jock itch, onychomycosis, dandruff, athlete's foot, sinusitis, otitis media, Methicillin-resistant Staphylococcus aureus (MRSA), staph, otitis media, swimmer's ear, and bacterial vaginosis. Additional systemic conditions that may be treated with preparations disclosed herein include systemic inflammation, such as eczema, e.g., adult and pediatric eczema, hives, idiopathic uriticaria, lichen planus, insect bites including allergic reactions to insect bites, e.g., mosquito and demodex folliculorum mite, reactions to poison ivy, itchiness, keratosis pilaris, laryngitis, pemphigus, psoriasis, rosacea, folliculitis and subtypes of folliculitis, hidradenitis supportiva, perioral dermatitis, lupus rash, seborrheic dermatitis, e.g., adult and infantile seborrheic dermatitis, acne, e.g., adolescent acne, adult acne, and cystic acne, diaper rash, occupational hand dermatitis, sunburn, and dermatomyositis. Additionally, preparations disclosed herein may be delivered or applied to treat certain cosmetic indications, including but not limited to, contact dermatitis, diaper odor, e.g., adult and pediatric, body odor, feminine odor, flaking, nail hardness, body odor, oily skin, razor burn, skin appearance, skit blotchiness, skin hydration, and sun spots. Preparations disclosed herein may be applied as a bug repellant or an antimicrobial agent.

### 11. Methods of Supporting a Microbiome of a Subject

There are provided methods of maintaining a microbiome of a subject. For example, methods of maintaining a community of microorganisms on a subject may include using a consumer product as described herein in a vicinity or environment of the subject. The methods may include maintaining a community of microorganisms on a surface of the consumer product. In certain embodiments, the methods may include introducing a biome-friendly consumer product to the subject.

Methods of maintaining a community of microorganisms on a surface may comprise applying a preparation comprising microorganisms to the surface or introducing the preparation comprising microorganisms to a subject. The preparation may be applied or introduced prior to applying the consumer product or the finished consumer product. The method may comprise applying a preparation comprising microorganisms to the surface or introducing the preparation comprising microorganisms to the subject subsequent to applying the consumer product or the finished consumer product.

The method may further comprise applying the preparation comprising microorganisms to the surface prior to applying the consumer product or the finished consumer product, wherein the preparation comprising microorganisms is applied between about one of the following ranges: about 1-5, 5-10, 10-20, 20-30, 30-40, 40-50, 50-60 minutes, 2-5, 5-10, 10-15, 15-20, 20-25 hours, 2-5, 5-10, 10-15, days, 3-4, 5-10 weeks prior to applying the consumer product or the finished consumer product.

The method may further comprise applying the preparation comprising microorganisms to the subject subsequent to applying the consumer product or the finished consumer product, wherein the preparation comprising microorganisms is applied between about one of the following ranges: about 1-5, 5-10, 10-20, 20-30, 30-40, 40-50, 50-60 minutes, 2-5, 5-10, 10-15, 15-20, 20-25 hours, 2-5, 5-10, 10-15, days, 3-4, 5-10 weeks subsequent to applying the consumer product or the finished consumer product.

The method may comprise not applying a non biome-friendly product or preparation prior to or subsequent to applying the preparation comprising microorganisms.

In the method and system embodiments of this disclosure, at least one of the preparation of microorganisms and the consumer product is applied to a pre-defined area of the surface. The pre-defined area of the surface may include a component of the surface or a substantial portion of the surface. The pre-defined area of the surface may be at least one of a portion of a subject including a head, e.g., a face, cheek, chin, eyelid, lip, nose, scalp, hair, forehead; neck; underarm; arm; hand; leg; foot; chest; abdomen region; buttocks; genital area; and back.

The methods may comprise a second or subsequent use of the consumer product in the environment of the subject. The second or subsequent use may be performed after a pre-determined period of time. The second or subsequent use may comprise a second or subsequent application of the consumer product to a surface. In some embodiments, the pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour. The pre-determined period of time may be less than 1 hour, for example, the pre-determined period of time may be 50 minutes, 45 minutes, 40 minutes, 35 minutes, 30 minutes, 25 minutes, 20 minutes, 15 minutes, 10 minutes, 5 minutes, 3 minutes, 2 minutes, or 1 minute. In certain instances, the pre-determined period of time may be less than 1 minute.

The methods may comprise applying a second or subsequent dose of the microorganisms to the surface or introducing a second or subsequent dose of the microorganisms to the subject. The second or subsequent dose may be introduced or applied after a pre-determined period of time. In some embodiments, the pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour. The pre-determined period of time may be less than 1 hour, for example, the pre-determined period of time may be 50 minutes, 45 minutes, 40 minutes, 35 minutes, 30 minutes, 25 minutes, 20 minutes, 15 minutes, 10 minutes, 5 minutes, 3 minutes, 2 minutes, or 1 minute. In certain instances, the pre-determined period of time may be less than 1 minute. The second or subsequent dose may be introduced or applied once it has been determined that a second or subsequent dose is needed, for example, to maintain the community of microorganisms.

### 12. Kits Comprising Biome-Friendly Consumer Products

According to certain aspects, there are provided kits comprising a consumer product as disclosed herein. For instance, there are provided kits comprising a biome-friendly consumer product. The consumer product may be disposed in an end-use container.

The disclosure provides compositions comprising microorganisms. The kit may comprise a preparation comprise microorganisms, e.g., beneficial bacteria, e.g., non-pathogenic bacteria, as disclosed herein. The kit may comprise a preparation comprise microorganisms, e.g., live microorganisms, e.g., a purified preparation of microorganisms. The compositions comprising microorganisms may be provided in a cosmetic or therapeutic product. The compositions comprising microorganisms may be provided in a consumer product. The compositions may comprise natural products comprising microorganisms.

The present disclosure provides, inter alia, compositions comprising ammonia oxidizing microorganisms, e.g., a preparation of ammonia oxidizing microorganisms, or a purified preparation of ammonia oxidizing microorganisms. The compositions comprising ammonia oxidizing microorganisms, e.g., a preparation of ammonia oxidizing microorganisms, or a purified preparation of ammonia oxidizing microorganisms may be provided in a cosmetic product or a therapeutic product. The compositions comprising ammonia oxidizing microorganisms may be provided in a consumer product. The compositions may comprise natural products comprising ammonia oxidizing microorganisms.

In some aspects, the disclosure provides compositions, e.g., preparations, with a defined number of species. For instance, in some embodiments, the disclosure provides compositions including 1, 2, 3, 4, 5, 6, 7, 8, 9, or 10 species of microorganisms, and no other types of organism. The preparation may be substantially free of other types of organisms.

In some embodiments, one or more other organisms besides the selected microorganism may be included in the preparation. For example, an organism of the genus selected from the group consisting of Lactobacillus, Streptococcus, Bifidobacter, and combinations thereof, may be provided in the preparation in addition to the selected microorganism

In some embodiments, the composition, e.g., preparation, provides conditions that support microorganism viability. The composition may promote microorganism growth and metabolism or may promote a dormant state (e.g., freezing) or storage state as described herein. For instance, the composition may promote ammonia oxidizing microorganism growth and metabolism or may promote a dormant state (e.g., freezing) or storage state as described herein, from which viable ammonia oxidizing microorganisms can be recovered. When the composition promotes growth or metabolism, it may contain water and/or nutrients that microorganisms consume. The preparation may comprise ammonia oxidizing microorganism nutrients, e.g., as ammonium ions, ammonia, urea, oxygen, carbon dioxide, or trace minerals.

Preparations may comprise between about between about 10⁸ to about 10¹⁴ CFU/L of the selected microorganism. The preparation may comprise at least about 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/L.

Preparations may comprise between about 10⁸ to about 10¹⁴ CFU/ml of the selected microorganism. The preparation may comprise at least about 10⁸, 10⁹, 10¹⁰, 10¹¹, 2×10¹¹, 5×10¹¹, 10¹², 2×10¹², 5×10¹², 10¹³, 2×10¹³, 5×10¹³, or 10¹⁴; or about 10⁸-10⁹, 10⁹-10¹⁰, 10¹⁰-10¹¹, 10¹¹-10¹², 10¹²-10¹³, or 10¹³-10¹⁴ CFU/ml.

In some embodiments, the preparation may comprise between about 0.1 milligrams (mg) to about 100 mg of the selected microorganism. In certain aspects, the preparation may comprise between about 50 mg and about 1000 mg of the selected microorganism. The preparation may comprise between about 0.1-0.5 mg, 0.2-0.7 mg, 0.5-1.0 mg, 0.5-2 mg, 0.5-5 mg, 2.5-5 mg, 2.5-7.0 mg, 5.0-10 mg, 7.5-15 mg, 10-15 mg, 15-20 mg, 15-25 mg, 20-30 mg, 25-50 mg, 25-75 mg, 50-75 mg, 50-100 mg, 75-100 mg, 100-200 mg, 200-300 mg, 300-400 mg, 400-500 mg, 500-600 mg, 600-700 mg, 700-800 mg, 800-900 mg, 900-1000 mg, 100-250 mg, 250-500 mg, 100-500 mg, 500-750 mg, 750-1000 mg, or 500-1000 mg.

In some embodiments, the preparation may comprise a mass ratio of the selected microorganism to an excipient, e.g., a pharmaceutically acceptable excipient, a commercially acceptable excipient, or a cosmetically acceptable excipient in a range of about 0.1 grams per liter to about 1 gram per liter. The preparation may comprise a mass ratio of microorganism to an excipient in a range of about 0.1-0.2, 0.2-0.3, 0.1-0.5, 0.2-0.7, 0.5-1.0, or 0.7-1.0 grams per liter.

In some embodiments, the preparation may be the selected microorganism in a buffer solution comprising, consisting essentially of, or consisting of disodium phosphate and magnesium chloride, for example, 50 mM Na₂HPO₄ and 2 mM MgCl₂. The preparation may be provided in a buffer at a pre-determined volume of, for example, between about 0.1 and about 100 fluid ounces, about 0.2 and about 50 fluid ounces, about 0.5 and about 25 fluid ounces, about 1.0 and about 10 fluid ounces, about 2.0 and about 7 fluid ounces, about 3 and about 5 fluid ounces. In some embodiments, the preparation may be provided in a container. The preparation may be provided in a container constructed to contain about 3.4 fluid ounces, or any other volume disclosed herein. The preparation may be in a form that may be capable of being aerosolized, sprayed or misted, i.e., in the form of a mist.

The microorganisms may be combined with one or more excipients, e.g., one or more pharmaceutically acceptable excipients, commercially acceptable excipients, or cosmetically acceptable excipients. In some embodiments, “pharmaceutically acceptable excipient” refers to a pharmaceutically-acceptable material, composition, or vehicle, such as a liquid or solid filler, diluent, solvent, or encapsulating material. In some embodiments, each excipient is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, Remington: The Science and Practice of Pharmacy, 21st ed.; Lippincott Williams & Wilkins: Philadelphia, Pa., 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, Fla., 2009.

In some embodiments, a commercially acceptable excipient refers to a commercially acceptable material, material, composition, or vehicle, such as a liquid or solid filler, diluent, solvent, or encapsulating material. In some embodiments, each excipient is commercially acceptable in the sense of being compatible with the other ingredients of a consumer product or formulation, and suitable for use in contact with the tissue or organ of humans and animals without excessive toxicity, irritation, allergic response, immunogenicity, or other problems or complications, commensurate with a reasonable benefit/risk ratio.

In some embodiments, a cosmetically acceptable excipient refers to a cosmetically acceptable material, composition, or vehicle, such as a liquid or solid filler, diluent, solvent, or encapsulating material. In some embodiments, each excipient is cosmetically acceptable in the sense of being compatible with the other ingredients of a cosmetic formulation, and suitable for use in contact with the tissue or organ of humans and animals without excessive toxicity, irritation, allergic response, immunogenicity, or other problems or complications, commensurate with a reasonable benefit/risk ratio.

The excipient, e.g., the pharmaceutically acceptable excipient, commercially acceptable excipient, or the cosmetically acceptable excipient may be provided in the containers and kits of the present disclosure, e.g., within a preparation of beneficial microorganisms, within an activator, or within one or more chambers, e.g., a first chamber, second chamber, or mixing chamber of the container.

While it is possible for the active ingredient, e.g., the microorganism, to be administered alone, in many embodiments it is present in a pharmaceutical formulation, preparation, or composition, in a commercial or consumer product, formulation, preparation, or composition, or a cosmetic formulation, preparation, or composition. Accordingly, this disclosure provides a pharmaceutical formulation (preparation or composition), a consumer product, e.g., device, formulation, preparation, or composition, or a cosmetic formulation (preparation or composition) comprising beneficial microorganisms and a pharmaceutically acceptable excipient, commercially acceptable excipient, or a cosmetically acceptable excipient. Pharmaceutical compositions, commercial products, and cosmetic compositions may take the form of a formulation as described below.

The pharmaceutical, commercial, and cosmetic formulations (e.g., preparations, or compositions) described herein may include those suitable for oral (e.g., by way of, or for the purposes of depositing in the gastrointestinal tract), parenteral (including subcutaneous, intradermal, intramuscular, intravenous, and intraarticular), inhalation (including fine particle dusts or mists which may be generated by means of various types of metered doses, pressurized aerosols, nebulizers or insufflators, and including intranasally (nasal) or via the lungs (pulmonary)), rectal and topical (including dermal, transdermal, transmucosal, buccal, sublingual, and intraocular) administration, although the most suitable route may depend upon, for example, the condition and disorder of the recipient. The preparations or formulations may be configured to be administered to the subject topically, orally, enterally, intranasally, parenterally, subcutaneously, ocularly, otically, or respiratorily.

The formulations (e.g., preparations or compositions) may conveniently be presented in unit dosage form and may be prepared by any of the methods known in the art of pharmacy or cosmetology. Typically, methods include the step of bringing the active ingredient (e.g., microorganism) into association with a pharmaceutical or a comestic carrier which constitutes one or more accessory ingredients. In general, the formulations are prepared by uniformly and intimately bringing into association the active ingredient with liquid carriers or finely divided solid carriers or both and then, if necessary, shaping the product into the desired formulation.

Formulations may be presented as discrete units such as capsules, cachets or tablets, each containing a predetermined amount of ammonia oxidizing bacteria; as a powder or granules; as a solution or a suspension in an aqueous liquid or a non-aqueous liquid; or as an oil-in-water liquid emulsion or a water-in-oil liquid emulsion. The active ingredient may also be presented as a bolus, electuary or paste. Various pharmaceutically acceptable carriers and their formulation are described in standard formulation treatises, e.g., Remington's Pharmaceutical Sciences by E. W. Martin. See also Wang, Y. J. and Hanson, M. A., Journal of Parenteral Science and Technology, Technical Report No. 10, Supp. 42:2 S, 1988.

The compositions, or preparations, can, for example, be administered in a form suitable for immediate release or controlled (extended) release. Suitable examples of sustained-release systems include suitable polymeric materials, for example semi-permeable polymer matrices in the form of shaped articles, e.g., films, or microcapsules; suitable hydrophobic materials, for example as an emulsion in an acceptable oil; or ion exchange resins. Controlled (sustained)-release systems may be administered orally; rectally; parenterally; intracistemally; intravaginally; intraperitoneally; topically, for example as a powder, ointment, gel, drop or transdermal patch; bucally; or as a spray.

Preparations for administration can be suitably formulated to give controlled release of microorganisms. For example, the formulations, preparations, or compositions may be in the form of particles comprising one or more of biodegradable polymers, polysaccharide jellifying and/or bioadhesive polymers, or amphiphilic polymers. These compositions exhibit certain biocompatibility features which allow a controlled release of an active substance. See U.S. Pat. No. 5,700,486. The preparation may comprise a controlled release material.

In certain instances in this disclosure sustained-release or control-release systems may be referred to as a barrier.

Exemplary compositions, e.g., as a preparation, may include suspensions which can contain, for example, microcrystalline cellulose for imparting bulk, alginic acid or sodium alginate as a suspending agent, methylcellulose as a viscosity enhancer, dicalcium phosphate, starch, magnesium stearate and/or lactose and/or other excipients, binders, extenders, disintegrants, diluents and lubricants, mannitol, lactose, sucrose and/or cyclodextrins. Also included in such formulations may be high molecular weight excipients such as celluloses (avicel) or polyethylene glycols (PEG). Such formulations can also include an excipient to aid mucosal adhesion such as hydroxy propyl cellulose (HPC), hydroxy propyl methyl cellulose (HPMC), sodium carboxy methyl cellulose (SCMC), maleic anhydride copolymer (e.g., Gantrez), and agents to control release such as polyacrylic copolymer (e.g. Carbopol 934). Lubricants, surfactants, glidants, flavors, coloring agents and stabilizers may also be added for ease of fabrication and use. The surfactant may be a zwitterionic surfactant, a non-ionic surfactant, or an anionic surfactant.

Surfactants may include one or more of the following, alone, or in combination with those listed, or other surfactants or surfactant-like compounds: cocamidopropyl betaine (ColaTeric COAB), polyethylene sorbitol ester (e.g., Tween 80), ethoxylated lauryl alcohol (RhodaSurf 6 NAT), sodium laureth sulfate/lauryl glucoside/cocamidopropyl betaine (Plantapon 611 L UP), sodium laureth sulfate (e.g., RhodaPex ESB 70 NAT), alkyl polyglucoside (e.g., Plantaren 2000 N UP), sodium laureth sulfate (Plantaren 200), Dr. Bronner's Castile soap, Dr. Bronner's baby soap, Lauramine oxide (ColaLux Lo), sodium dodecyl sulfate (SDS), polysulfonate alkyl polyglucoside (PolySufanate 160 P), sodium lauryl sulfate (Stepanol-WA Extra K) and combinations thereof. Dr. Bronner's Castile soap and baby soap comprises water, organic coconut oil, potassium hydroxide, organic olive oil, organic fair deal hemp oil, organic jojoba oil, citric acid, and tocopherol. Castile soaps, e.g., Dr. Bronner's Castile soap, and many natural and baby soaps are comprised of water, organic or non-organic animal or vegetable oil, sodium or potassium hydroxide, organic olive oil, organic fair deal hemp oil, organic jojoba oil, citric acid, and tocopherol.

Surfactants may include Sodium Laurylglucosides Hydroxypropylsulfonate (Suga®nate 160NC), lauramidopropyl betaine (Cola®Teric LMB); Cocamidopropyl hydroxysultaine (Cola®Teric CBS); disodium cocoamphodiacetate (Cola®Teric CDCX-LV); sodium laurylglucosides hydroxypropyl phosphate (Suga®Fax D12).

Surfactants may include sodium lauroyl methyl isethionate (Iselux® LQ-CLR-SB); sodium methyl cocoyl taurate (Pureact WS Conc.); Aqua (and) Sodium Lauroyl Methyl Isethionate (and) Cocamidopropyl Betaine (and) Sodium Cocoyl Isethionate (and) Sodium Methyl Oleoyl Taurate (Iselux®SFS-SB)

In some embodiments, surfactants may be used with ammonia oxidizing microorganisms in amounts that allow nitrite production to occur. In some embodiments, the preparation may have less than about 0.01% to about 10% of surfactant. In some embodiments, the concentration of surfactant used may be between about 0.0001% and about 10%. In some embodiments, the preparation may be substantially free of surfactant.

In some embodiments, the formulation, e.g., preparation, may include other components that may enhance effectiveness of microorganisms, or enhance a treatment or indication. In some embodiments, the formulation, e.g., preparation, may include other components that may enhance effectiveness of ammonia oxidizing microorganisms.

In some embodiments, a chelator may be included in the preparation. A chelator may be a compound that may bind with another compound, e.g., a metal. The chelator may provide assistance in removing an unwanted compound from an environment, or may act in a protective manner to reduce or eliminate contact of a particular compound with an environment, e.g., microorganisms, e.g. a preparation of beneficial microorganisms, e.g., an excipient.

Formulations (e.g., preparations) may also contain anti-oxidants, buffers, bacteriostats that prevent the growth of undesired bacteria, solutes, and aqueous and non-aqueous sterile suspensions which may include suspending agents and thickening agents. The formulations may be presented in unit-dose or multi-dose containers, for example sealed ampoules and vials, and may be stored in a freeze-dried (lyophilised) condition requiring only the addition of a sterile liquid carrier, for example saline or water-for-injection, immediately prior to use. Extemporaneous solutions and suspensions may be prepared from powders, granules and tablets of the kind previously described. Exemplary compositions include solutions or suspensions which can contain, for example, suitable non-toxic, pharmaceutically acceptable diluents or solvents, such as mannitol, 1,3-butanediol, water, Ringer's solution, an isotonic sodium chloride solution, or other suitable dispersing or wetting and suspending agents, including synthetic mono- or diglycerides, and fatty acids, including oleic acid, or Cremaphor. An aqueous carrier may be, for example, an isotonic buffer solution at a pH of from about 3.0 to about 8.0, a pH of from about 3.5 to about 7.4, for example from 3.5 to 6.0, for example from 3.5 to about 5.0. Useful buffers include sodium citrate-citric acid and sodium phosphate-phosphoric acid, and sodium acetate/acetic acid buffers. The composition in some embodiments does not include oxidizing agents.

Excipients that can be included are, for instance, proteins, such as human serum albumin or plasma preparations. If desired, the pharmaceutical composition, e.g., a preparation, may also contain minor amounts of non-toxic auxiliary substances, such as wetting or emulsifying agents, surfactants, preservatives, and pH buffering agents and the like, for example sodium citrate, sodium acetate or sorbitan monolaurate. In some embodiments, excipients, e.g., a pharmaceutically acceptable excipient or a cosmetically acceptable excipient, may comprise an anti-adherent, binder, coat, disintegrant, filler, flavor, color, lubricant, glidant, sorbent, preservative, or sweetener. In some embodiments, the preparation may be substantially free of excipients.

Exemplary compositions for aerosol administration include solutions in saline, which can contain, for example, benzyl alcohol or other suitable preservatives, absorption promoters to enhance bioavailability, and/or other solubilizing or dispersing agents. Conveniently in compositions for aerosol administration the microorganisms may be delivered in the form of an aerosol spray presentation from a pressurized pack or a nebulizer, with the use of a suitable propellant, e.g., dichlorodifluoro-methane, trichlorofluoromethane, dichlorotetrafluoroethane, carbon dioxide, nitrogen, or other suitable gas. In the case of a pressurized aerosol the dosage unit can be determined by providing a valve to deliver a metered amount. Capsules and cartridges of e.g., gelatin can be formulated to contain a powder mix of the microorganisms and a suitable powder base, for example lactose or starch. In certain embodiments, microorganisms are administered as an aerosol from a metered dose valve, through an aerosol adapter also known as an actuator. Optionally, a stabilizer is also included, and/or porous particles for deep lung delivery are included (e.g., see U.S. Pat. No. 6,447,743). The composition or preparation may be in a form that may be capable of being aerosolized, sprayed or misted, i.e., in the form of a mist. The preparation of microorganisms may be microorganisms in a buffer solution comprising, consisting essentially of, or consisting of disodium phosphate and magnesium chloride, for example, 50 mM Na₂HPO₄ and 2 mM MgCl₂. The preparation of ammonia oxidizing microorganisms may be ammonia oxidizing microorganisms in a buffer solution comprising, consisting essentially of, or consisting of disodium phosphate and magnesium chloride, for example, 50 mM Na₂HPO₄ and 2 mM MgCl₂.

Formulations may be presented with carriers such as shea or cocoa butter, synthetic glyceride esters or polyethylene glycol. Such carriers are typically solid at ordinary temperatures, but liquefy and/or dissolve at body temperature to release the microorganisms.

Exemplary compositions for topical administration include a topical carrier such as Plastibase (mineral oil gelled with polyethylene). In some aspects, the composition, e.g., preparation, and/or excipient may be in the form of one or more of a liquid, a solid, or a gel. For example, liquid suspensions may include, but are not limited to, water, saline, phosphate-buffered saline, or an ammonia oxidizing storage buffer.

Gel formulations may include, but are not limited to agar, silica, polyacrylic acid (for example Carbopol®), carboxymethul cellulose, starch, guar gum, alginate, clays, or chitosan. In some embodiments, the formulation, e.g., preparation, may be supplemented with an ammonia source including, but not limited to one or more of ammonia, ammonium ions, e.g., ammonium chloride or ammonium sulfate, and urea.

In some embodiments, an ammonia oxidizing bacteria composition, e.g., preparation, is formulated to improve NO penetration into the skin. A gel-forming material such as KY jelly or various hair gels would present a diffusion barrier to NO loss to ambient air, and so improve the skin's absorption of NO. The NO level in the skin will generally not greatly exceed 20 nM/L because that level activates GC and would cause local vasodilatation and oxidative destruction of excess NO.

It should be understood that in addition to the ingredients particularly mentioned above, the formulations, e.g., preparations, as described herein may include other agents conventional in the art having regard to the type of formulation in question.

The formulation, e.g., preparation, e.g., composition may be provided in a container, delivery system, or delivery device, having a weight, including or not including the contents of the container, that may be less than about 50, 100, 200, 300, 400, 500, 600, 700, 800, 900, 1000, 1500, or 2000 grams.

Suitable unit dosage formulations are those containing an effective dose, as hereinbefore recited, or an appropriate fraction thereof, of microorganisms.

A therapeutically effective amount of the microorganism may be administered as a single pulse dose, as a bolus dose, or as pulse doses administered over time. Thus, in pulse doses, a bolus administration of microorganisms is provided, followed by a time period wherein microorganisms are not administered to the subject, followed by a second bolus administration. In specific, non-limiting examples, pulse doses are administered during the course of a day, during the course of a week, or during the course of a month.

In some embodiments, a preparation, e.g., a formulation, e.g., a composition, may be applied for a pre-determined number of days. This may be based, for example, at least in part, on the severity of the condition or disease, the response to the treatment, the dosage applied and the frequency of the dose. For example, the preparation may be applied for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days. In certain aspects, the preparation may be applied for about 16 days.

In some embodiments, a preparation, e.g., a formulation, e.g., a composition, may be applied a pre-determined number of times per day. This may be based, for example, at least in part, on the severity of the condition or disease, the response to the treatment, the dosage applied and the frequency of the dose. For example, the preparation may be applied 1, 2, 3, 4, 5, 6, 7, 8, 9, 10, 11, 12, 13, 14, 15, 16, 17, 18, 19, 20, 21, 22, 23, 24 times per day.

In some embodiments, the preparation may be applied one time per day. In other embodiments, the preparation may be applied two times per day. In some embodiments, the preparation may be applied a first pre-determined amount for a certain number of days, and a second pre-determined amount for a certain subsequent number of days. In some embodiments, the preparation may be applied for about 16 days.

In some embodiments, the preparation is administered for about 1-3, 3-5, 5-7, 7-9, 5-10, 10-14, 12-18, 12-21, 21-28, 28-35, 35-42, 42-49, 49-56, 46-63, 63-70, 70-77, 77-84, 84-91 days, e.g., for about 1 month, for about 2 months, for about 3 months. In some embodiments, the preparation is administered for an indefinite period of time, e.g, greater than one year, greater than 5 years, greater than 10 years, greater than 15 years, greater than 30 years, greater than 50 years, greater than 75 years.

Other components may be added to pharmaceutical formulations, e.g., preparations, consumer preparations, or cosmetic preparations as selected by one skilled in the art such as, for example, water, mineral oil, coloring agent, perfume, aloe, glycerin, sodium chloride, sodium bicarbonate, pH buffers, UV blocking agents, silicone oil, natural oils, vitamin E, herbal concentrates, lactic acid, citric acid, talc, clay, calcium carbonate, magnesium carbonate, zinc oxide, starch, urea, and erythorbic acid, or any other excipient known by one of skill in the art, including those disclosed herein.

In some embodiments, the preparation may be disposed in, or provided as, a powder, cosmetic, cream, stick, aerosol, salve, wipe, or bandage.

In some embodiments, microorganism is associated with a cream. The cream may be a fluid comprising a thickening agent, and generally has a consistency that allows it to be spread evenly on the skin. Exemplary creams include moisturizing lotion, face cream, and body lotion.

In some embodiments, the microorganism is associated with a stick. A stick is typically a solid that, when placed in contact with a surface, transfers some of the stick contents to the surface. Exemplary sticks include deodorant stick, lipstick, lip balm in stick form, and sunscreen applicator sticks.

In some embodiments, the microorganism is associated with an aerosol. An aerosol is typically a colloid of fine solid particles or fine liquid droplets, in a gas such as air. Aerosols may be created by placing the microorganism (and optionally carriers) in a vessel under pressure, and then opening a valve to release the contents. The container may be designed to only exert levels of pressure that are compatible with microorganism viability. For instance, the high pressure may be exerted for only a short time, and/or the pressure may be low enough not to impair viability. Examples of consumer uses of aerosols include for sunscreen, deodorant, perfume, hairspray, and insect repellant.

In some embodiments, the microorganism is associated with a salve. A salve may be a topically applied agent with a liquid or cream-like consistency, intended to protect the skin or promote healing. Examples of salves include burn ointments and skin moisturizers.

In some embodiments, the microorganism is associated with a wipe. A wipe may be a flexible material suitable for topically applying a liquid or cream onto skin. The wipe may be, e.g., paper-based or cloth based. Exemplary wipes include tissues and wet wipes.

The compositions comprising microorganisms may also comprise one or more of a moisturizing agent, deodorizing agent, scent, colorant, insect repellant, cleansing agent, or UV-blocking agent.

For instance, the moisturizing agent may be an agent that reduces or prevents skin dryness. Exemplary moisturizing agents include humectants (e.g., urea, glycerin, alpha hydroxy acids and dimethicone) and emollients (e.g., lanolin, mineral oil and petrolatum). Moisturizing agents may be included, e.g., in ammonia oxidizing bacteria-containing creams, balms, lotions, or sunscreen.

A deodorizing agent may be an agent that reduces unwanted odors. A deodorizing agent may work by directly neutralizing odors, preventing perspiration, or preventing the growth of odor-producing bacteria. Exemplary deodorizing agents include aluminum ions (e.g., aluminum chloride or aluminum chlorohydrate), cyclomethicone, talc, baking soda, essential oils, mineral ions, hops, and witch hazel. Deodorizing agents are typically present in spray or stick deodorants, and can also be found in some soaps and clothing.

An insect repellant may be an agent that can be applied to surfaces (e.g., skin) that discourage insects and other arthropods from lighting on the surface. Insect repellants include DEET (N,N-diethyl-m-toluamide), p-menthane-3,8-diol (PMD), icaridin, nepetalactone, citronella oil, neem oil, bog myrtle, dimethyl carbate, Tricyclodecenyl allyl ether, and IR3535 (3-[N-Butyl-N-acetyl]-aminopropionic acid, ethyl ester).

A cleansing agent may be an agent that removes dirt or unwanted bacteria from a surface. Exemplary cleansing agents include bar soaps, liquid soaps, foam soaps, and surface cleaners.

A UV-blocking agent may be an agent that can be applied to a surface to reduce the amount of ultraviolet light the surface receives. A UV-blocking agent may block UV-A and/or UV-B rays. A UV blocking agent can function by absorbing, reflecting, or scattering UV. Exemplary UV-blocking agents include absorbers, e.g., homosalate, octisalate (also called octyl salicylate), octinoxate (also called octyl methoxycinnamate or OMC), octocrylene, oxybenzone, and avobenzone, and reflectors (e.g., titanium dioxide and zinc oxide). UV-blocking agents are typically present in sunscreens, and can also be found in skin creams and some cosmetics.

In some embodiments, microorganisms are associated with a conditioner. Conditioner generally refers to a substance that can be applied to a surface, for example, to alter the condition of the surface.

In some embodiments, microorganisms are associated with cloth. Cloth generally refers to a flexible material suitable to be made into clothing, e.g., having enough material strength to withstand everyday motion by a wearer. Cloth can be fibrous, woven, or knit; it can be made of a naturally occurring material or a synthetic material. Exemplary cloth materials include cotton, flax, wool, ramie, silk, denim, leather, nylon, polyester, and spandex, and blends thereof.

In some embodiments, microorganisms are associated with yarn. Yarn generally refers to a long, thin spun flexible material that is suitable for knitting or weaving. Yarn can be made of, e.g., wool, cotton, polyester, and blends thereof.

In some embodiments, microorganisms are associated with thread. Thread generally refers to a long, thin spun flexible material that is suitable for sewing. Thread generally has a thinner diameter than yarn. Thread can be made of, e.g., cotton, polyester, nylon, silk, and blends thereof. Microorganisms can be associated with a fabric or textile.

In some embodiments, microorganisms are associated with a solid material, for example, polymers, metals, ceramics, paper, wood, tile, or glass.

In some embodiments, the microorganisms are associated with a consumer product. The consumer product comprising is packaged. The packaging may serve to compact the product or protect it from damage, dirt, or degradation. The packaging may comprise, e.g., plastic, paper, cardboard, or wood. In some embodiments the packaging is impermeable to microorganisms, e.g., bacteria. In some embodiments the packaging is permeable to oxygen and/or carbon dioxide.

The preparation may comprise a concentration or amount of microorganisms in order to alter, e.g., reduce or increase, an amount, concentration or proportion of a selected microorganism, e.g., genus of bacteria, on a surface. The microorganism may be non-pathogenic. In some embodiments, the microorganism may be pathogenic, or potentially pathogenic.

In some embodiments, the preparation of ammonia oxidizing microorganisms may be in a growth state. A growth state may be provided by exposing ammonia oxidizing microorganisms to an environment that may promote growth. The growth state may be a state, e.g., ammonia oxidizing microorganisms in an environment that allows immediate availability of ammonia oxidizing microorganisms to convert ammonium ions (NH₄⁺) to nitrite (NO₂⁻). The growth state may comprise providing ammonia oxidizing microorganisms in an environment having a pH of greater than about 7.6. The growth state may also comprise providing ammonia oxidizing microorganisms in an environment having ammonia, ammonium ions, and/or urea, trace minerals and sufficient oxygen and carbon dioxide.

In some embodiments, the preparation of ammonia oxidizing microorganisms may be in a polyphosphate loading state, wherein the state or the environment, e.g., a media, e.g., a culture media, e.g., a growth media, may have a pH of less than about 7.4. Levels of at least one of ammonia, ammonium ions, and urea may be between about 10 micromolar and 200 millimolar. Levels of trace materials may be between 0.1 micromolar iron and 20 micromolar iron. Levels of oxygen may be between about 5% and 100% oxygen saturation. Levels of carbon dioxide may be between/less than about zero and 200 ppm, and phosphate levels greater than about 10 micromolar. The purpose of the polyphosphate loading state is to provide AOM with ammonia and oxygen such that ATP can be produced, but to deny them carbon dioxide and carbonate such that they are unable to use that ATP to fix carbon dioxide and instead use that ATP to generate polyphosphate which may be stored.

In some embodiments, the preparation of ammonia oxidizing microorganisms may be in a storage state. A storage state may be defined as ammonia oxidizing microorganisms in an environment in which they may be stored to be later revived. The storage state may be a state, e.g., ammonia oxidizing microorganisms in an environment that allows availability of ammonia oxidizing microorganisms after being revived, e.g., after being place in an environment promoting a growth state for a pre-determined period of time. The pre-determined period of time for revival may be less than 72 hours. For example, the pre-determined period of time may be less than about 75 hours, or less than about 72 hours. The pre-determined period of time may at least partially based on a period time of about 0.2-10 times, 0.3-5 times, 0.5-3 times, 0.5-1.5 times, or 0.5 to 1 times the doubling time for the ammonia oxidizing microorganisms. The pre-determined period of time may be at least partially based on a period of time of about one doubling time for the ammonia oxidizing microorganisms. The pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour. The pre-determined period of time may be between about 5 minutes and 5 hours. The pre-determined period of time may be about 5-10 minutes, 10-15 minutes, 15-20 minutes, 20-25 minutes, 25-30 minutes, 30-45 minutes, 45-60 minutes, 60 minutes-1.5 hours, 1.5 hours-2 hours, 2 hours-2.5 hours, 2.5 hours-3 hours, 3 hours-3.5 hours, 3.5 hours-4 hours, 4 hours-4.5 hours, 4.5 hours-5 hours. In some embodiments, the pre-determined period of time may be about 2 hours.

The storage state may comprise providing ammonia oxidizing microorganisms in an environment having a pH of less than about 7.4. The storage state may also comprise providing ammonia oxidizing microorganisms in an environment having ammonia, ammonia ions, and/or urea, trace minerals, oxygen, and low concentrations of carbon dioxide.

Microorganisms may be kept in a storage state. Storage may also be accomplished by storing at 4° C. for up to several months. The storage buffer in some embodiments may comprise 50 mM Na₂HPO₄-2 mM MgCl₂ (pH 7.6).

In some embodiments, microorganisms may be cyropreserved. A 1.25 ml of microorganism mid-log culture may be added to a 2 ml cryotube and 0.75 ml of sterile 80% glycerol. Tubes may be shaken gently, and incubate at room temperature for 15 min to enable uptake of the cryoprotective agents by the cells. The tubes may be directly stored in a −80° C. freezer for freezing and storage.

For resuscitation of cultures, frozen stocks may be thawed on ice for 10-20 minutes, and then centrifuged at 8,000×g for 3 minutes at 4° C. The pellet may be washed by suspending it in 2 ml of medium followed by another centrifugation at 8,000×g for 3 minutes at 4° C. to reduce potential toxicity of the cryoprotective agents. The pellet may be resuspended in 2 ml of medium, inoculated into 50 ml of medium containing 50 mM NH₄⁺, and incubated in dark at 30° C. by shaking at 200 rpm.

In some embodiments, the preparation of ammonia oxidizing bacteria may comprise ammonia oxidizing microorganisms in a storage state and/or ammonia oxidizing microorganisms in a polyphosphate loading state, and/or ammonia oxidizing microorganisms in a growth state.

In some embodiments, upon actuation of the container, delivery system or device, ammonia oxidizing microorganisms in a storage state or a polyphosphate loading state may be mixed with an activator. The activator may be in a form to provide a pH of at least about 7.6. The activator may be in a form to provide an environment having ammonia, ammonium ions, and/or urea, trace minerals and sufficient oxygen and carbon dioxide. The activator may revive or at least partially revive the ammonia oxidizing microorganisms in a storage state or a polyphosphate loading state to a growth state. The time that it takes to revive the ammonia oxidizing microorganisms from a storage state (or a polyphosphate loading state) may be a pre-determined period of time. For example, the pre-determined period of time may be less than about 75 hours, or less than about 72 hours. The pre-determined period of time may at least partially based on a period time of about 0.2-10 times, 0.3-5 times, 0.5-3 times, 0.5-1.5 times, or 0.5 to 1 times the doubling time for the ammonia oxidizing microorganisms. The pre-determined period of time may be at least partially based on a period of time of about one doubling time for the ammonia oxidizing microorganisms. The pre-determined period of time may be between about 8 hours and 12 hours. The pre-determined period of time may be about 10 hours. The pre-determined time may be less than about 75 hours, 72 hours, 70 hours, 68 hours, 65 hours, 60 hours, 55 hours, 50 hours, 45 hours, 40 hours, 35 hours, 30 hours, 25 hours, 20 hours, 15 hours, 10 hours, 5 hours, 4 hours, 3, hours, 2 hours, or 1 hour.

In some embodiments, the container may comprise ammonia oxidizing microorganisms in a growth state, and in at least one of a storage state and a polyphosphate loading state, so as to provide ammonia oxidizing microorganisms immediately to an environment to begin converting at least one of ammonia, ammonium ions, and urea to nitrite, while allowing for revival of the ammonia oxidizing microorganisms in at least one of the storage state and the polyphosphate loading state over a period of time. This may allow for a controlled release of the stored ammonia oxidizing microorganisms over a period of time.

Without wishing to be bound by theory, by maintaining ammonia oxidizing microorganisms under conditions or in an environment of low carbon dioxide, with sufficient oxygen and ammonia, they may accumulate polyphosphate for a pre-determined period, e.g., for a period of about one doubling time, e.g., for about 8-12 hours, e.g., for about 10 hours. The ammonia oxidizing microorganisms may accumulate sufficient polyphosphate to extend their storage viability, storage time, and accelerate their revival. This may occur with or without the addition of buffer and ammonia.

The presence of sufficient stored polyphosphate may allow the ammonia oxidizing microorganisms the ATP resources to maintain metabolic activity even in the absence of ammonia and oxygen, and to survive insults that would otherwise be fatal.

The process of oxidation of ammonia to generate ATP has two steps. The first step is the oxidation of ammonia to hydroxylamine by ammonia monoxoygenase (Amo), followed by the conversion of hydroxylamine to nitrite by hydroxylamine oxidoreductase (Hao). Electrons from the second step (conversion of hydroxylamine to nitrite) are used to power the first step (oxidation of ammonia to hydroxylamine).

If an ammonia oxidizing microorganisms do not have hydroxylamine to generate electrons for Amo, then hydroxylamine is not available for Hao. For example, acetylene irreversibly inhibits the enzyme crucial for the first step in the oxidation of ammonia to nitrite, the oxidation of ammonia to hydroxylamine. Once AOM are exposed to acetylene, Amo is irreversibly inhibited and new enzyme must be synthesized before hydroxylamine can be generated. In a normal consortium biofilm habitat, AOM may share and receive hydroxylamine form other AOM (even different strains with different susceptibilities to inhibitors) and so the biofilm tends to be more resistant to inhibitors such as acetylene than an individual organism. AOM can use stored polyphosphate to synthesize new Amo, even in the absence of hydroxylamine.

Any embodiment, preparation, composition, or formulation of microorganisms discussed herein may comprise, consist essentially of, or consist of optionally axenic microorganisms.

The kit may further comprise a probiotic. The probiotic may be in the form or a preparation or composition. The probiotic may include a preparation comprising one or more microorganisms. The probiotic may include a community of microorganisms. In some embodiments, the microbial composition of the probiotic is unknown. The probiotic may be combined with the preparation comprising microorganisms or packaged separately. The probiotic may be administered to provide a community of microorganisms for the microorganism preparation.

The kit may further comprise a prebiotic. The prebiotic may be in the form of a preparation or composition. The prebiotic may include one or more substance that stimulates the growth of microorganisms. For example, the prebiotic may include a substance that is metabolized by one or more microorganism, e.g., beneficial microorganism, optionally to produce a byproduct, e.g., a beneficial byproduct. The prebiotic may be combined with the preparation comprising microorganisms or packaged separately.

The kit may further comprise instructions for use. The instructions may be provided, for example, in printed form on the packaging (exterior or interior packaging), as a package insert, or in digital format. The instructions may include methods of use of one or more kit components, as disclosed herein. In some embodiments, the instructions are directed to combined use of more than one component of the kit, as disclosed herein. The instructions may be directed to combined use of the consumer product, e.g., biome-friendly consumer product, and the preparation including microorganisms. In some embodiments, the combined use of the consumer product and the preparation may impart a cosmetic or therapeutic effect in a subject.

### Prophetic Example

### Biome-Friendly Ingredient and Formulae Testing Method

Recovery of Ammonia Oxidizing Bacteria (AOB) N. eutropha D23 after Treatment with Various Excipients

N. eutropha D23 AOB suspensions may be obtained from a continuous culture system. The N. eutropha D23 AOB culture may be harvested by centrifugation at 10,000×g for 15 minutes at 20° C. The harvested cells may be washed in a microorganism storage solution (50 mM Na₂HPO₄-2 mM MgCl₂, pH 7.6) and suspended in storage solution at a final optical density (OD₆₀₀) of 5.0 (˜10¹⁰ cells/ml) prior to storing at 4° C. To determine the effect of each excipient, the N. eutropha D23 AOB cell suspension may be diluted to a final optical density (OD₆₀₀) of 0.5 (˜10⁹ cells/ml) in 10 ml medium supplemented with ammonium (NH₄⁺) containing various concentrations of the excipient (0 to 100%). Control cultures may be supplemented with an equal volume of excipient diluent only. The cultures may be incubated at 30° C. At 1 min, 10 min & 60 min time points, 1 ml of the cultures may be collected, centrifuged at 17,000×g for 3 minutes. The supernatant may be used to measure nitrite by Griess reagents. The bacterial pellet obtained may be washed in medium, suspended in 10 ml medium supplemented with NH₄⁺ and incubated at 30° C. shaking at 150 rpm (upright position) on an orbital shaker. Recovery of N. eutropha D23 AOB cells from treatment with excipient may be monitored for 24-48 hr by determining OD₆₀₀ values & nitrite accumulation in samples collected at 24 hr intervals.

Depending on the ingredient, excipient, or composition tested, the concentration of nitrite measured may allow for identification of a biome-friendly ingredient, excipient, or composition. In certain embodiments, only nitrite production of greater than 1000 micromolar, measured at the end of a 48 hour period would be indicative of a biome-friendly ingredient, excipient, or composition. In other embodiments, nitrite production of greater than 100 micromolar, measured at the end of a 48 hour period would be indicative of a biome-friendly ingredient, excipient, or composition. In other embodiments, nitrite production of greater than 10 micromolar, measured at the end of a 48 hour period would be indicative of a biome-friendly ingredient, excipient, or composition.

Recovery identified as “+++” is indicative of nitrite production of greater than 1000 micromolar, measured at the end of a 48 hour period of time, and may provide for a biome-friendly ingredient, excipient or composition. Recovery identified as “++” is indicative of nitrite production between about 100 micromolar and 1000 micromolar, at the end of a 48 hour period of time, and may provide for a biome-friendly ingredient, excipient or composition. Recovery identified as “+” is indicative of nitrite production between about 10 and 100 micromolar, at the end of a 48 hour period of time, and may provide for a biome-friendly ingredient, excipient or composition. No recovery (−) is indicative of none or substantially no nitrite production measured, for example, less than 10 micromolar nitrite production in a 48 hour period of time, which would in most instances would not be indicative of a biome-friendly ingredient, excipient or composition.

This procedure may apply to water soluble or miscible ingredients. For oil or immiscible ingredients or formulae, the test substance may first be adsorbed to a carrier bead or mesh. This may be rinsed in buffer and then the beads or mesh may be placed in the bacterial suspension and incubated for the specified time period. The bare bead or mesh may be incubated as a control. At the end of the test exposure period, the supernatant bacterial suspension may be tested for activity and viability as per the soluble test methodology, discussed above.

For cleansers 1 and 2, testing may be done at 10% and 25% because certain cleaners, e.g., laundry detergent, are typically diluted with water during use.

Exemplary compositions tested may have the follow composition of ingredients:

092214-004 Foaming Rose Scented Cleanser

| | Con- | Possible | |
|---|---|---|---|
| Component | centration | Concentration | Ingredient |
| Natrasol 300-CS | 0.25% | 0.10-0.5% | Viscosity |
| (Hydroxyethyl | | | modifier |
| Cellulose) | | | |
| Cocamidopropyl Betaine | 20.00% | 5.0-12.0% | Cleanser/Surfactant |
| Decyl Glucoside | 4.0% | 0.0-8.0% | Cleanser/Surfactant |
| Botanimoist AMG | 2.0% | 0.0-4.0% | Humectant |
| (Glycerine + Apple | | | |
| ext) | | | |
| Natural Rose Hydrosol | 5.0% | 0.0-10.0% | Fragrance |

092014-003 Foaming Unscented Cleanser

| | Con- | Possible | |
|---|---|---|---|
| Component | centration | Concentration | Ingredient |
| Natrasol 300-CS | 0.25% | 0.10-0.5% | Viscosity |
| (Hydroxyethyl | | | modifier |
| Cellulose) | | | |
| Cocamidopropyl Betaine | 8.00% | 5.0-12.0% | Cleanser/Surfactant |
| Decyl Glucoside | 4.0% | 0.0-8.0% | Cleanser/Surfactant |
| Botanimoist AMG | 2.0% | 0.0-4.0% | Humectant |
| (Glycerine + Apple ext) | | | |

092214-003 Cleanser 1

| | | Possible | |
|---|---|---|---|
| Component | Concentration | Concentration | Ingredient |
| Natrasol 300-CS | 2.00% | 1.0-2.5% | Viscosity modifier |
| (Hydroxyethyl | | | |
| Cellulose) | | | |
| Cocamidopropyl | 20.00% | 15.0-30.0% | Cleanser/Surfactant |
| Betaine | | | |
| Decyl Glucoside | 10.0% | 0.0-15.0% | Cleanser/Surfactant |
| Botanimoist AMG | 2.0% | 0.0-4.0% | Humectant |
| (Glycerine + Apple | | | |
| ext) | | | |

092214-001 Cleanser 2

| | Con- | Possible | |
|---|---|---|---|
| Component | centration | Concentration | Ingredient |
| Natrasol 300-CS | 2.00% | 0.5-2.5% | Viscosity modifier |
| (Hydroxyethyl | | | |
| Cellulose) | | | |
| Cocamidopropyl Betaine | 20.00% | 10.0-30.0% | Cleanser/Surfactant |
| Decyl Glucoside | 5.0% | 0.0-10.0% | Cleanser/Surfactant |
| Botanimoist AMG | 2.0% | 0.0-4.0% | Humectant |
| (Glycerine + Apple | | | |
| extract) | | | |

092214-002 Surface Conditioner

| | | Possible | |
|---|---|---|---|
| | Con- | Concentration | |
| Component | centration | Range | Ingredient |
| Natrasol 300-CS | 2.0% | 0.10-0.5% | Viscosity modifier |
| (Hydroxyethyl | | | |
| Cellulose) | | | |
| Myritol 312 C8-10 | 2.5% | 1.0-5.0% | Conditioner |
| Triglycerides | | | |
| Lamisoft ® PO 65 | 2.5% | 0.0-5.0% | Lipid layer |
| (Coco-Glucoside and | | | enhancer |
| Glyceryl Oleate) | | | |
| Polysorbate-80 | 1.0% | 0.0-3.0% | Emulsifier |
| Natural Rose Water | 5.0% | 0.0-10.0% | Fragrance |

Nitrite production after 1 minute of incubation in the foaming cleanser, both scented and unscented may be achieved. After longer periods of incubation (10 min, and 60 min), nitrite production may be achieved, but may generally be less than that after 1 minute of incubation. The nitrite production achieved would correlate to the amount of AOB recovered in the test sample.

Nitrite production after 1 minute of incubation in cleanser 1 may be achieved and may generally be comparable to the control test sample for both 10% and 25% concentrations (the 25% concentration sample may have less nitrite production). With increased incubation time, less nitrite production may be achieved. The nitrite production achieved may correlate to the amount of AOB recovered in the test sample.

Nitrite production after 1 minute of incubation in cleanser 2 may be achieved and may generally be comparable to the control test sample for both 10% and 25% concentrations (the 25% concentration sample may have less nitrite production). With increased incubation time, less nitrite production may be achieved. The nitrite production achieved may correlate to the amount of AOB recovered in the test sample.

Nitrite production after 1 minute of incubation in the surface conditioner may be achieved and may generally be comparable to the control test sample. With increased incubation time, similar nitrite production may be achieved. The nitrite production achieved may correlate to the amount of AOB recovered in the test sample.

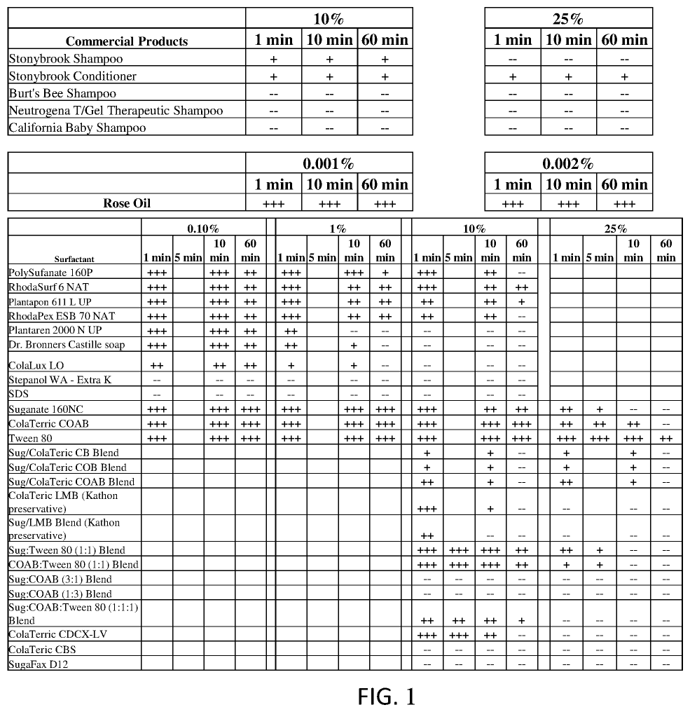

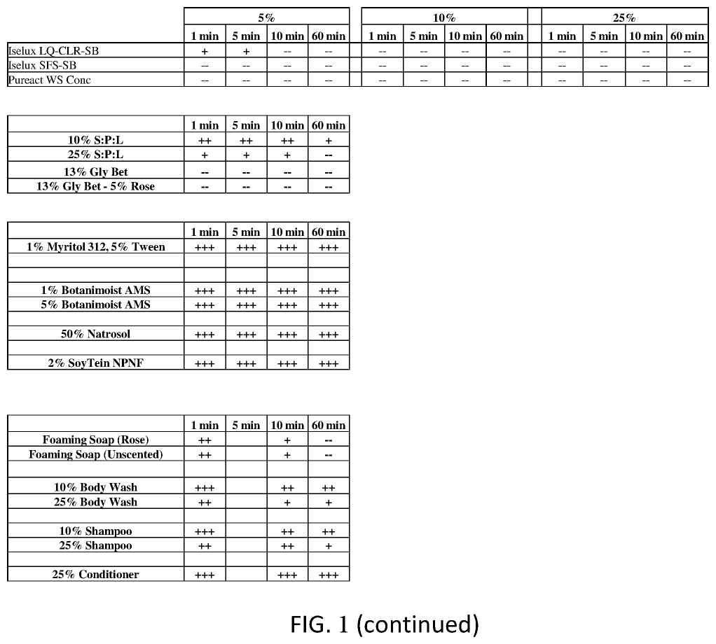

With regard to the Table in FIG. 1, the following tables describe the components of some compositions which may be tested:

### 10% S:P:L and 25% S:P:L (the Percentages Refer to Testing Conditions)

| | | Suggested | |
|---|---|---|---|
| | Concentration | Concentration | |
| Component | Tested | Range | Description |
| Suganate 160 | 20.0% | 10.0-30.0% | Biobased |
| Sodium Laurylglucosides | | | surfactant- |
| Hydroxypropylsulfonate | | | cleanser |
| Plantacare 818 (Coco | 2.5% | 1.0-5.0% | Biobased |
| glucoside) | | | surfactant- |
| | | | cleanser |
| Lamisoft ® PO 65 | 3.0% | 0.0-5.0% | Lipid layer |
| (Coco-Glucoside and | | | enhancer |
| Glyceryl Oleate) | | | |

“13% Gly Bet” (in this Case the 13% Refers to the Formula Itself)

| | | Suggested | |
|---|---|---|---|
| | Concentration | Concentration | |
| Component | Tested | Range | Description |
| Sodium Cocoyl- | 10.0% | 5.0-20.0% | Biobased |
| Glycinate | | | surfactant- |
| | | | cleanser |
| Cocamidopropyl | 3.0% | 5.0-12.0% | Cleanser/surfactant |
| Betaine | | | |

### 13% Gly Bet-5% Rose

| | | Suggested | |
|---|---|---|---|
| | Concentration | Concentration | |
| Component | Tested | Range | Description |
| Sodium Cocoyl- | 10.0% | 5.0-20.0% | Biobased |
| Glycinate | | | surfactant- |
| | | | cleanser |
| Cocamidopropyl | 3.0% | 5.0-12.0% | Cleanser/ |
| Betaine | | | surfactant |
| Natural Rose Hydrosol | 5.0% | 0.0-10% | Fragrance |

FIG. 1 shows the degree to which particular surfactants, or blends of surfactants are expected to be biome-friendly. The symbols are associated with nitrite production as compared to the control.

Recovery identified as “+++” is indicative of nitrite production of greater than 1000 micromolar, measured at the end of a 48 hour period of time. Recovery identified as “++” is indicative of nitrite production between about 100 micromolar and 1000 micromolar, at the end of a 48 hour period of time. Recovery identified as “+” is indicative of nitrite production between about 10 and 100 micromolar, at the end of a 48 hour period of time. No recovery (−) is indicative of none or substantially no nitrite production measured, for example, less than 10 micromolar nitrite production in a 48 hour period of time.

### ANNEX VI—ASEAN COSMETIC DOCUMENTS

### Annex VI—Part 1—List of Preservative Allowed for Use in Cosmetic Products

ANNEX VI-PART 1
LIST OF PRESERVATIVES ALLOWED

| | | | | Conditions of use and |
|---|---|---|---|---|
| Reference | | Maximum authorized | Limitations and | warnings which must be |
| Number | Substance | concentration | requirements | printed on the label |
| a | b | c | d | e |
| 1 | Benzoic acid (CAS No. | Rinse off products, except | | |
| | 65-85-9), and its | oral care products: 2.6% | | |
| | sodium salt (CAS | (acid) | | |
| | No. 532-32-1) | oral care products: 1.7% | | |
| | | (acid) | | |
| | | Leave-on products: 0.5% | | |
| | | (acid) | | |
| 1a | Salts of benzoic acid | 0.8% (acid) | | |
| | other than that listed | | | |
| | under reference numer 1 | | | |
| | and esters of benzoic | | | |
| | acid | | | |
| 2 | Propionic acid and its | 2% (acid) | | |
| | salts | | | |
| 3 | Salicylic acid and its | 0.5% (acid) | Not to be used | Not to be used for |
| | salts (+) [symbol] | | in preparation | children under three |
| | | | for children | years of age [symbol] |
| | | | under three | |
| | | | years of age, | |
| | | | except for | |
| | | | shampoos | |
| 4 | Sorbic acid (hexa-2,4- | 0.6% (acid) | | |
| | denoic acid) and its | | | |
| | salts | | | |
| 5 | Formaldehyde and | 0.2% (except for products | Prohibited in | |
| | paraformaldehyde(+) [symbol] | for oral hygiene) | aerosol | |
| | | 0.1% (for oral hygiene) | dispensers(sprays) | |
| | | expressed as free | | |
| | | formaldehyde | | |
| 6 | Entry moved or deleted | 0.2% expressed as phenol | | |
| 7 | Biphenyl-2-ol(o-phenylphenol) | | | |
| | and its salts | | | |
| 8 | Zinc pyrithicone (+) [symbol] (CAS | Hair products: 1.0% | Rinse off products only. | |
| | No 13403-41-7) | Other products: 0.5% | Not for use in products | |
| | | | for oral hygience | |
| 9 | Inorganic sulphites and | 0.2% expressed as free CO₂ | | |
| | hydrogensulphites(+) [symbol] | | | |
| 10 | Entry moved or deleted | | | |
| 11 | Chlorobutanol(INN) | 0.5% | Prohibited in aerosol | Contains chlorobutanol |
| | | | dispensers (sprays) | |
| 12 | 4-Hydroxybenzoic acid and | 0.4% (acid) for 1 ester. | | |
| | its salts and esters | 0.8% (acid) for mixtures | | |
| | | of esters | | |
| 13 | 3-Acetyl-6-methylpyran-2,4 | 0.5% (acid) | Prohibition in aerosol | |
| | (3H)-dione | | dispensers (sprays) | |
| | (Dehydroacetic acid) and | | | |
| | its salts | | | |
| 14 | Formic acid and its sodium | 0.5% (expressed as acid) | | |
| | salt | | | |
| 15 | 3,3′-Dibromo-4,4′- | 0.1% | | |
| | hexamethylenedioxydi- | | | |
| | benzamidine | | | |
| | (Dibromohexamidine) and its | | | |
| | salts (Including isethionate) | | | |
| 16 | Thiomersal(INN) | 0.007% (of Hg) | For eye make-up and | Contains thiomersal |
| | | If mixed with other | eye make-up remover | |
| | | mercurial compounds | only | |
| | | authorized by this | | |
| | | Directive, the maximum | | |
| | | concentration of Hg remains | | |
| | | fixed at 0.007% | | |
| 17 | Phenulmercuric salts including | 0.007% (of Hg) | For eye make-up and | Contains phenylmercuric |
| | borate) | If mixed with other | eye make-up remover | compounds |
| | | mercurial compounds | only | |
| | | authorized by this | | |
| | | Directive, the maximum | | |
| | | concentration of Hg | | |
| | | remains fixed at 0.007% | | |
| 18 | Undec-10-anoic acid and | 0.2% (acid) | | |
| | its salts(+) | | | |
| 19 | Hexetidine(INN) | 0.1% | | |
| 20 | 5-Bromo-6-nitro-1,3 | 0.1% | Rinse-off products only | |
| | dioxane | | Avoid formation of | |
| | | | ribosamines | |
| 21 | Bronopol(INN) | 0.1% | Avoid formation of | |
| | | | nitrosamines | |
| 22 | 2,4-Dichlorobenzyl alcohol | 0.15% | | |
| 23 | Triclocarban(INN) (+) [symbol] | 0.2% | Purity criteria: 3,3′,4,4′- | |
| | | | Tetrachloroazobenzene | |
| | | | <1 ppm, 3,3′,4,4′-Tetra | |
| | | | chloroaoxybenzene | |
| | | | <1 ppm | |
| 24 | 4-Chloro-m-oresol | 0.2% | Prohibited in products | |
| | | | intended to come into | |
| | | | contact with mucous | |
| | | | membranes | |
| 25 | Triclosaryl(INN) (+) | 0.3% | | |
| 26 | 4-Chloro-3,5-xylenol | 0.5% | | |
| 27 | 3,3′-Bis(1-hydroxymethyl-2,5- | 0.6% | | |
| | dioxoimidazolidin- | | | |
| | 4-yl)-1,1′-methylenediures | | | |
| | (‘Imidazolidinyl urea’) | | | |
| 28 | Poly(1-hexamethylenebiguande | 0.3% | | |
| | hydrochloride) | | | |
| 29 | 2-Phenoxyethanol | 1.0% | | |
| 30 | Hexamethylenetetramine | 0.15% | | |
| | (methanamine) (INN) | | | |
| 31 | Methanamine 3- | 0.2% | | |
| | chloroalkylochloride (INNM) | | | |
| 32 | 1-(4-Chlorophenoxy)-1- | 0.5% | | |
| | (Imidazol-1-yl)-3,3- | | | |
| | dimethylbutan-2-one) (+) | | | |
| 33 | 1,3-Bis(hydroxymethyl)-5,5- | 0.6% | | |
| | dimethylimidazolidine-2,4- | | | |
| | dione) | | | |
| 34 | Benzyl alcohol(+) [symbol] | 1% | | |
| 35 | 1-Hydroxy-4-methyl-8-(2,4- | 1% | Risne-off products | |
| | trimethylpentyl)-2 | 0.5% | For other products | |
| | pyridon and its | | | |
| | monoethanolamine salt(+) | | | |
| 36 | Entry deleted | | | |
| 37 | 6,6-Dibromo-4,4-dichloro- | 0.1% | | |
| | 2,2′-methylene-diphenol | | | |
| | (Bromochlorophen) | | | |
| 38 | 4-isopropyl-m-oresol | 0.1% | | |
| 39 | Mixture of 5-Chloro-2-methyl- | 0.0015% (of a mixture in | | |
| | isothiazol-3(2H)- one and 2- | the ratio 3:1 of 5-Chloro- | | |
| | MethylIsothiazol-3(2H)-one | 2-methyl-isothiazol-3(2H)- | | |
| | with magnesium chloride and | one and 2-methylisothiazol- | | |
| | magnesium nitrate) | 3(2H)-one) | | |
| 40 | 2-Benzyl-4-chlorophenol | 0.2% | | |
| | (chlorophene) | | | |
| 41 | 2-Chloroacetamide | 0.3% | | Contains |
| | | | | chloroacetamide |
| 42 | Chlorhexidine(INN) and its | 0.3% expressed as | | |
| | digluconate, diacetate and | chlorhexidine | | |
| | dihydrochloride(+) | | | |
| 43 | 1-Phenoxypropan-2-ol(+) [symbol] | 1.0% | Only for rinse-off | |
| | | | products | |
| 44 | Alkyl (C12-C22) trimethyl | 0.1% | | |
| | ammonium, bromide | | | |
| | and chloride) (+) | | | |
| 45 | 4,4-Dimethyl-1,3-oxazolidine | 0.1% | The pH of the finished | |
| | | | product must not be | |
| | | | lower than 6 | |
| 46 | N-(Hydroxymethyl)-N- | 0.5% | | |
| | (dihydromethyl-1,3-dioxide- | | | |
| | 2,5-imidazolinidyl-4)-N- | | | |
| | (hydroxymethyl) urea) | | | |
| 47 | 1,6-Di(4-amidinophenoxy)-n- | 0.1% | | |
| | hexane (Hexamidine) and | | | |
| | its salts (including isethionate | | | |
| | and p-hydroxybenzoate(+) | | | |
| 48 | Glutaraldehyde | 0.1% | Prohibited in aerosols | Contains glutaraldehyde |
| | (Pentane-1,5-diol) | | (sprays) | (where glutaraldehyde |
| | | | | concentration in the |
| | | | | finished product exceeds |
| | | | | 0.06%) |
| 49 | 5-Ethyl-3,7-dioxa-1- | 0.3% | Prohibited in oral | |
| | azabicyclo [3,3,0] octane | | hygiene products | |
| | | | and in products | |
| | | | intended to come into | |
| | | | contact with mucous | |
| | | | membranes | |
| 50 | 3-(n-chlorophenoxy)-propane- | 0.3% | | |
| | 1,2-diol (chloropenesin) | | | |
| 51 | Sodium hydroxymethylamino | 0.5% | | |
| | acetate (Sodium | | | |
| | hydroxymethylglycinate | | | |
| 52 | Silver chloride deposited on | 0.004% calculated as | 20% AgCl (w/w) on | |
| | titanium chloride | AgCl | TiO₂ Prohibited in | |
| | | | products for children | |
| | | | under three years of | |
| | | | age, in oral hygiene | |
| | | | products and in | |
| | | | products intended for | |
| | | | application around | |
| | | | the eyes and on the | |
| | | | lips | |
| 53 | BenzethoniumChloride | 0.1% | (a) Rinse-off products | |
| | (INCI) | | (b) Leave on products | |
| | | | other than for oral use. | |
| 54 | Benzalkonium chloride, | 0.1% calculated as | | Avoid contact with the |
| | bromide and saccharinate(+) [symbol] | Benzalkonium | | eyes |
| 55 | Benzylhemformal | 0.15% | Only for products to be | |
| | | | removed by rinsing | |
| 56 | Iodopropynylbutylcarbamate | (a) rinse-off | Not to be used in oral | (a) Not to be used for |
| | (IPEC): 3-Iodo-2- | products: 0.02% | hygiene and lip care | children under three |
| | propynylbutylcarbamate | (b) leave-on | products | years of age (**) |
| | CAS No: 65406-53-8 | products: 0.01% | (a) Not to be used in | (b) Not to be used for |
| | | except in | preparations for | children under three |
| | | deodorants & | children under three | years of age(***) |
| | | antiperspirants: | years of age, | |
| | | 0.0075% | except in bath | |
| | | | products/shower gels | |
| | | | and shampoos. | |
| | | | (b) Not to be used in | |
| | | | body lotion and body | |
| | | | cream (*) | |
| | | | Not to be used in | |
| | | | preparations for | |
| | | | children under three | |
| | | | years of age. | |
| 57 | Methylisothiazolinone(INCI) | 0.01% | | |
| 58 | Ethyl LauroylArginateHCI | 0.4% | Not to be used in Sp | |
| | (INCI) (+) [symbol] Ethyl-N α- | | products, oral products | |
| | dodecanoyl-L-arginate | | and spray products. | |
| | hydrochloride CAS No | | | |
| | EC No 434-630-6 | | | |

[symbol]

indicates data missing or illegible when filed

With regard to Annex VI:
1. Preservatives are substances which may be added to cosmetic products for the primary purpose of inhibiting the development of micro-organisms in such products.
2. The substances marked with the symbol (+) may also be added to cosmetic products in concentration other than those laid down in this ANNEX for other purposes apparent from the presentation of the products, e.g. as deodorants in soaps or as anti-dandruff agents in shampoo.
3. Other substances used in the formulation of cosmetic products may also have anti-microbial properties and thus help in the preservation of the products, as, for instance, many essential oils and some alcohols. These substances are not included in the ANNEX.
4. For the purposes of this list
   “Salts” is taken to mean: salts of the cations sodium, potassium, calcium, magnesium, ammonium, and ethanolamines; salts of the anions chloride, bromide, sulphate, acetate.
   “Esters” is taken to mean: esters of methyl, ethyl, propy, isopropyl, butyl, isobutyl, phenyl.
5. All finished products containing formaldehyde or substances in this ANNEX and which release formaldehyde must be labeled with the warning “contains formaldehyde” where the concentration of formaldehyde in the finished product exceeds 0.05%.

1. A biome-friendly consumer product comprising ammonia oxidizing microorganisms (AOM), wherein said biome-friendly consumer product is supportive of a microbiome of a subject.

2. The product of claim 1, wherein said consumer product, in the absence of a treatment, e.g., sterilization treatment or the addition of a preservative, supports microbe growth, e.g., bacterial or fungal growth, e.g., as measured by U.S.P. 51, Antimicrobial Effectiveness Testing or U.S.P. 62, Microbial Examination of Nonsterile Products.

3. The product of claim 1, wherein said consumer product, if exposed to challenge with a microbe, e.g., a bacterium or fungus, will support growth of said microbe, e.g., as determined by U.S.P. 51, Antimicrobial Effectiveness Testing or U.S.P. 62, Microbial Examination of Nonsterile Products.

4. The product of claim 1, wherein the consumer product allows the microbiome of a subject to be maintained.

5. The product of claim 1, wherein the consumer product is substantially free of microorganisms.

6. (canceled)

7. The product of claim 1, wherein the consumer product is formulated or treated to provide a health benefit or otherwise support a healthy lifestyle.

8. (canceled)

9. The product of claim 1, wherein the consumer product is formulated, treated, or configured, e.g., structured, to support engraftment of the AOM.

10.-28. (canceled)

29. The product of claim 1, wherein the consumer product is substantially free of a preservative.

30. The product of claim 29, wherein the consumer product has less than about 500 ppb of a preservative listed in Annex VI, e.g., less than 500 ppb of a preservative listed in Annex VI.

31. The product of claim 1, wherein the consumer product is irradiated or otherwise sterilized.

32.-33. (canceled)

34. The product of claim 1, wherein the consumer product is treated to favor at least one pre-determined species of microorganism, e.g., the AOM.

35. The product of claim 1, wherein the consumer product is treated to suppress growth or reproduction of at least one pre-determined species of microorganism, to have anti-fungal properties, or to have anti-viral properties.

36.-37. (canceled)

38. The product of claim 1, wherein the consumer product is associated with a deterioration-based or a biome-compatible-based expiration date.

39.-44. (canceled)

45. A kit comprising:
the biome-friendly consumer product of claim 1 and a preparation comprising ammonia oxidizing microorganisms (AOM).

46.-49. (canceled)

50. The kit of claim 45, wherein the preparation comprises between about 1×10³ CFU/mL to about 1×10¹⁴ CFU/mL AOM.

51. (canceled)

52. The kit of claim 45, wherein the AOM comprise ammonia oxidizing bacteria (AOB).

53.-54. (canceled)

55. The kit of claim 52, wherein the AOM comprise Nitrosomonas, Nitrosococcus, Nitrosospira, Nitrosocystis, Nitrosolobus, Nitrosovibrio, and combinations thereof.

56. The kit of claim 55, wherein the AOM is Nitrosomonas eutropha (N. eutropha).

57. The kit of claim 56, wherein the AOM is N. eutropha D23, having ATCC accession number PTA-121157.

58.-59. (canceled)

60. The kit of claim 45, wherein the preparation comprises AOM in a buffer solution, e.g., aqueous buffer solution, comprising disodium phosphate and magnesium chloride, for example, 50 mM Na₂HPO₄ and 2 mM MgCl₂ in water.

61.-64. (canceled)

65. A method of supporting a microbiome of a subject, comprising:
introducing the consumer product of claim 1 to the subject; and
introducing a source of ammonia oxidizing microorganisms (AOM) to the subject.

66.-71. (canceled)

72. A method of making a biome-friendly consumer product, comprising:
selecting a first component from a list of biome-friendly components;
selecting a second component from a list of biome-friendly components; and
integrating the first and second components with a source of ammonia oxidizing microorganisms (AOM), thereby making a biome-friendly consumer product.

73. The method of claim 72, further comprising:
providing the first and second components;
providing the source of ammonia oxidizing microorganisms (AOM); and
determining if the product is biome-friendly.

74. (canceled)

76. The method of claim 72, further comprising determining whether the product will support growth of a microbe, e.g., a bacterium or fungus.

77. The method of claim 76, comprising determining whether the product will support growth of a microbe, e.g., a bacterium or fungus, by U.S.P. 51, Antimicrobial Effectiveness Testing or U.S.P. 62, Microbial Examination of Nonsterile Products.

78. A method of evaluating a consumer product comprising ammonia oxidizing microorganisms (AOM), comprising:
contacting a portion or aliquot of the consumer product with a test organism; and
evaluating an effect of the consumer product on the test organism,
wherein evaluating comprises evaluating the effect of the consumer product on an ability of the test organism to breakdown organic waste or produce beneficial byproducts.

79. A method of rendering a consumer product biome-friendly, comprising:
applying a biome-friendly composition to the consumer product, and
applying a preparation comprising ammonia oxidizing microorganisms (AOM) to the consumer product.

80.-84. (canceled)

85. A method of certifying a consumer product as being biome-friendly, comprising:
providing a consumer product comprising ammonia oxidizing microorganisms (AOM);
determining if the product is biome-friendly by exposing the product to challenge with a microbe, e.g., a bacterium or fungus; and
certifying the product as being biome-friendly.

86.-88. (canceled)

89. The method of claim 85, further comprising contacting the consumer product with a test organism and evaluating the effect of the consumer product on an ability of the test organism to breakdown organic waste or produce beneficial byproducts.

90. A kit comprising:
the biome-friendly consumer product according to claim 1,
a preparation comprising a test organism comprising ammonia oxidizing microorganisms (AOM), and
instructions to evaluate the effect of the consumer product on the test organism.

91. (canceled)

92. The kit of claim 90, further comprising a reagent to detect nitrite production.
